(19) United States
(12) Patent Application Publication
(10) Pub. No.: US 2020/0190541 A1
(43) Pub. Date: Jun. 18, 2020

(54) METHODS FOR REDUCING DNA-INDUCED CYTOTOXICITY AND ENHANCING GENE EDITING IN PRIMARY CELLS

(71) Applicant: Precision BioSciences, Inc., Durham, NC (US)

(72) Inventor: Mark Johnson, Durham, NC (US)

(73) Assignee: Precision BioSciences, Inc., Durham, NC (US)

(21) Appl. No.: 16/609,150

(22) PCT Filed: Apr. 30, 2018
(86) PCT No.: PCT/US2018/030239

§ 371 Date: Oct. 28, 2019

Related U.S. Application Data
(60) Provisional application No. 62/491,740, filed on Apr. 28, 2017.

(51) Int. Cl. C12N 15/90; C12N 9/22; C12N 15/85; C12N 15/113; C07K 14/725; C07K 14/435; C07K 16/28; C12N 5/0783; G01N 33/50

(52) U.S. Cl. CPC C12N 15/907; C12N 9/22; C12N 15/85; C12N 15/113; C07K 14/7051; C07K 14/435; C07K 2319/00; C12N 5/0636; G01N 33/5005; C12N 2800/80; C12N 2310/14; C07K 2317/622; C12N 2510/00; C07K 16/2896

(57) ABSTRACT

An obstacle in the use of many primary cells is the significant cytotoxicity observed following transfection with circular or linearized DNA. Methods are provided for improving the survival of any primary cell following transfection with circular or linearized DNA. The methods of the invention are also useful for improving gene editing by endonucleases in DNA-transfected primary cells, and for improving the efficiency of targeted insertion of exogenous sequences into the genome of DNA-transfected primary cells.

### FIELD OF THE INVENTION

The invention relates to the field of molecular biology and recombinant nucleic acid technology. In particular, the invention relates to methods for genetic modification of eukaryotic primary cells.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 24, 2017, is named 2000706_00186US1.txt, and is 1,870 bytes in size.

### BACKGROUND OF THE INVENTION

Primary cells are a critical reagent in both pre-clinical research and in the development of downstream therapeutic products for the treatment of human disease. The heterogeneity in response to genetic or chemical manipulation of primary cells, as well as their in vitro and in vivo functionality upon such manipulation, more directly correlates with the potential applicability of any proposed therapeutic in human subjects. Within the context of cancer, modification of primary human T cells is an essential step in the production of cellular immunotherapies such as CAR T cells, which are prepared from primary human T cells. The manufacturing of such cells is done through the use of a viral delivery vector, such as lentivirus or adeno-associated virus, due to the enhanced ability of these systems to provide functional downstream CAR T cells upon transduction. (Gill, S. et anon., Immunol. Rev. 263, 68-89 (2015), Costello, E. et al., Gene Ther. 7, 596-604 (2000)) The use of such viral delivery systems, however, is limited by the amount of DNA that can be packaged within these viral vectors for delivery and is less cost-effective and more time-consuming to make compared to circular plasmid or linearized DNA constructs alone. (Li, C., et al., Cancer Gene Ther. 12, 913-925 (2005), Miller, A. D., Hum. Gene Ther. 1, 5-14 (1990)) Moreover, although the use of such systems has been widely approved for use in human applications and clinical trials, the inclusion of viral vectors for manufacturing requires an enhanced level of oversight. (Gill, S. et anon., Immunol. Rev. 263, 68-89 (2015)) Transfection with circular plasmid or linearized DNA alone is hindered by the significant toxicity and cell death associated with its delivery. (Jensen, M. C. et al., Mol. Ther. J. Am. Soc. Gene Ther. 1, 49-55 (2000), Ebert, O. et al., Gene Ther. 4, 296-302 (1997)) In addition, the remaining edited cells would require multiple rounds of expansion to acquire the number of cells that would be of therapeutic value to a patient. Such expansion can potentially result in detrimental changes in cellular phenotype, thereby limiting the in vivo effectiveness of such cells upon transfer. Therefore, the identification of methods that limit cell death upon DNA transfection of primary cells would represent a significant advancement in the field of primary cell editing.

In eukaryotic cells, DNA is localized within the nucleus and mitochondria. Therefore, the presence of DNA within the cytoplasm is recognized as a “danger signal” and results in the initiation of an innate immune response. One such pathway inherent to recognition of cytoplasmic DNA is the cGAS-STING-TBK1-IRF3 pathway, which upon activation results in the production of type I interferons and subsequent activation of interferon stimulatory genes (FIG. 1). (Cai, X., et al., Mol. Cell 54, 289-296 (2014), Stetson, D. B., et anon., Immunity 24, 93-103 (2006)) The activation of this pathway can occur either in response to invasion of the cell by foreign pathogens (Ishikawa, H., et al., Nature 461, 788-792 (2009)) or in the context of self-recognition during anti-tumor (Woo, et al., Immunity 41, 830-842 (2014)) or auto-immune responses. (Gall, A. et al., Immunity 36, 120-131 (2012), Ahn, J. et al., J. Immunol. Baltim. Md. 1950 193, 4634-4642 (2014))

Cyclic GMP-AMP synthase (cGAS) is a cytosolic protein that becomes activated in the presence of DNA. Once bound, cGAS undergoes a conformational change that triggers its ability to catalyze the synthesis of cGMP-AMP (cGAMP) from available ATP and GTP. (Li, X. et al., Immunity 39, 1019-1031 (2013), Sun, L., et al, Science 339, 786-791 (2013), Zhang, X. et al., Cell Rep. 6, 421-430 (2014)) cGAMP is a cyclic molecule composed of one adenine monophosphate (AMP) and one guanine monophosphate (GMP) characterized by a 2′-5′ and 3′-5′ linkage resulting in the formation of two phosphodiester bonds. (Gao, P. et al., Cell 153, 1094-1107 (2013), Zhang, X. et al., Mol. Cell 51, 226-235 (2013), Ablasser, A. et al., Nature 498, 380-384 (2013)) This isomer, known as 2′3′-cGAMP, then functions as a downstream messenger, trafficking to the endoplasmic reticulum (ER) where it binds to the ER-resident protein stimulator of interferon genes (STING). (Ishikawa, H. et anon., Nature 455, 674-678 (2008), Zhong, B. et al., Immunity 29, 538-550 (2008)) STING is a 379 amino acid protein that is comprised of four transmembrane domains that anchor it to the ER membrane where is exists as a dimer on the surface of the ER. (Gao, P. et al., Cell 154, 748-762 (2013), Yin, Q. et al., Mol. Cell 46, 735-745 (2012), Shu, C., et al., Nat. Struct. Mol. Biol. 19, 722-724 (2012)) Binding of 2′3′-cGAMP to STING results in a conformational change and relocation of STING from the ER membrane to the perinuclear Golgi. (Saitoh, T. et al., Proc. Natl. Acad. Sci. U.S.A 106, 20842-20846 (2009), Dobbs, N. et al., Cell Host Microbe 18, 157-168 (2015)) Once there, STING recruits and activates the kinase TANK-binding kinase 1 (TBK1), which is characterized by the formation of large punctate structures. (Saitoh, T. et al., Proc. Natl. Acad. Sci. U.S.A 106, 20842-20846 (2009)) Activated TBK1 subsequently phosphorylates and activates the transcription factor interferon regulatory factor 3 (IRF3). (Tanaka, Y. et anon., Sci. Signal. 5, ra20, Fitzgerald, K. A. et al., Nat. Immunol. 4, 491-496 (2003), Sharma, S. et al., Science 300, 1148-1151 (2003)) The phosphorylated IRF3 then dimerizes and enters the nucleus, resulting in the downstream activation of type I interferons, inflammatory cytokines, and functions as a positive feedback for expression of the cGAS gene itself. (Ma, F. et al., J. Immunol. Baltim. Md. 1950 194, 1545-1554 (2015))

Collectively, the cGAS-STING-TBK1-IRF3 pathway has been shown to play a pivotal role in several key aspects of cellular immunity, including responses to pathogens (Ishikawa, H., et al., Nature 461, 788-792 (2009)), tumor surveillance (Woo, S.-R. et al., Immunity 41, 830-842 (2014)), and autoimmune disease. (Gall, A. et al., Immunity 36, 120-131 (2012), Ahn, J. et al., J. Immunol. Baltim. Md. 1950 193, 4634-4642 (2014)). Additionally, activation of STING by pharmacological agonists has recently been explored as a potential therapeutic approach in certain cancers. (Roberts, Z. J. et al., J. Exp. Med. 204, 1559-1569 (2007), Corrales, L. et al., Cell Rep. 11, 1018-1030 (2015))

However, a role for the cGAS-STING-TBK1-IRF3 pathway in primary cell survival following DNA transfection has not been previously reported. Indeed, the inventors are the first to demonstrate that inhibition of IRF3 signaling, either by inhibition of IRF3 directly or by inhibition of upstream effectors such as cGAS, STING, or TBK1, can inhibit DNA-induced cytotoxicity in transformed primary cells. Thus, the methods of the invention significantly advance the art by allowing for simple, fast, and cost-effective modification of primary cells using DNA templates without the need for viral transduction or other techniques.

### SUMMARY OF THE INVENTION

An obstacle in the use of many primary cells is the significant cytotoxicity observed following transfection with circular or linearized DNA. The inventors have discovered that this DNA-induced cytotoxicity can be abrogated or reduced by inhibiting intracellular signaling through the Interferon Regulatory Factor 3 (IRF3) signaling pathway. The inventors further discovered that inhibition of IRF3 signaling can increase the number of gene-edited cells, and the percentage of gene-edited cells (i.e., increased gene editing efficiency), when an endonuclease is introduced into primary cells to target a recognition sequence on a chromosome of the cell. Moreover, the inventors have discovered that inhibition of IRF3 signaling can increase the number of cells comprising a targeted insertion of an exogenous sequence of interest into an endonuclease cleavage site, as well as the percentage of cells exhibiting targeted insertion (i.e., increased insertion frequency).

Inhibition of IRF3 signaling can be achieved in a number of ways, including small molecule inhibition of IRF3 directly, and/or inhibition of upstream effectors of IRF3, such as cGAS, STING, and/or TBK1. Inhibition can also be achieved by reducing or downregulating IRF3 protein expression by any method known in the art, and/or reducing or downregulating protein expression of upstream effectors of IRF3, including TBK1, STING, and/or cGAS.

In specific embodiments, the method is particularly useful in primary human T cells, transformed with a plasmid or linear DNA template comprising an exogenous nucleic acid sequence encoding a chimeric antigen receptor (CAR). Thus, in one embodiment, primary human T cells with reduced IRF3 signaling are nucleofected to introduce: (a) a nucleic acid (e.g., an mRNA) encoding an engineered endonuclease (e.g., meganuclease), and (b) plasmid or linearized DNA comprising a CAR coding sequence that is flanked by homology arms, such that the CAR coding sequence is inserted into a chromosome of the cell at the cleavage site induced by the engineered endonuclease.

Thus, in one aspect, a method is provided for reducing cytotoxicity associated with DNA transfection in primary eukaryotic cells, the method comprising: (a) reducing interferon regulatory factor 3 (IRF3) signaling in the primary eukaryotic cells; and, (b) transfecting a DNA template into the primary eukaryotic cells, wherein the transfected primary eukaryotic cells exhibit improved survival (i.e., increase in viable cell number) when compared to control cells.

In another aspect, a method is provided for increasing the number of gene-edited primary eukaryotic cells following DNA transfection, the method comprising: (a) reducing interferon regulatory factor 3 (IRF3) signaling in the primary eukaryotic cells; (b) transfecting a DNA template into the primary eukaryotic cells; and (c) introducing an endonuclease, or a nucleic acid encoding an endonuclease, into the primary eukaryotic cells, wherein the endonuclease recognizes and cleaves a recognition sequence present in the genome of the primary eukaryotic cells, and wherein the number of transformed primary eukaryotic cells exhibiting gene editing is increased compared to control cells.

In another aspect, a method is provided for increasing gene editing frequency in primary eukaryotic cells following DNA transfection, the method comprising: (a) reducing interferon regulatory factor 3 (IRF3) signaling in the primary eukaryotic cells; (b) transfecting a DNA template into the primary eukaryotic cells; and (c) introducing an endonuclease, or a nucleic acid encoding an endonuclease, into the primary eukaryotic cells, wherein the endonuclease recognizes and cleaves a recognition sequence present in the genome of the primary eukaryotic cells, and wherein the percentage of transformed primary eukaryotic cells exhibiting gene editing is increased compared to control cells.

In another aspect, a method is provided for increasing the number of primary eukaryotic cells comprising targeted insertion of an exogenous sequence of interest into the genome following DNA transfection, the method comprising: (a) reducing interferon regulatory factor 3 (IRF3) signaling in the primary eukaryotic cells; (b) transfecting a DNA template into the primary eukaryotic cells, wherein the DNA template comprises an exogenous sequence of interest; and (c) introducing an endonuclease, or a nucleic acid encoding an endonuclease, into the primary eukaryotic cells, wherein the endonuclease recognizes and cleaves a recognition sequence present in the genome of the primary eukaryotic cells to produce a cleavage site, and wherein the exogenous sequence of interest is flanked by homology arms having homology to regions upstream and downstream of the cleavage site resulting in targeted insertion of the exogenous sequence of interest into the cleavage site by homologous recombination, and wherein the number of transformed primary eukaryotic cells comprising targeted insertion of the exogenous sequence of interest is increased compared to control cells.

In another aspect, a method is provided for increasing insertion frequency of an exogenous sequence of interest into the genome in primary eukaryotic cells following DNA transfection, the method comprising: (a) reducing interferon regulatory factor 3 (IRF3) signaling in the primary eukaryotic cells; (b) transfecting a DNA template into the primary eukaryotic cells, wherein the DNA template comprises an exogenous sequence of interest; and (c) introducing an endonuclease, or a nucleic acid encoding an endonuclease, into the primary eukaryotic cells, wherein the endonuclease recognizes and cleaves a recognition sequence present in the genome of the primary eukaryotic cells to produce a cleavage site, and wherein the exogenous sequence of interest is flanked by homology arms having homology to regions upstream and downstream of the cleavage site resulting in targeted insertion of the exogenous sequence of interest into the cleavage site by homologous recombination, and wherein the percentage of transformed primary eukaryotic cells comprising targeted insertion of the exogenous sequence of interest is increased compared to control cells.

In some embodiments of the methods, the DNA template can be a single-stranded DNA template or a double-stranded DNA template. In particular embodiments, the DNA template can be a linearized DNA template or a plasmid DNA template.

In some embodiments of the methods, the primary eukaryotic cells can be maintained in culture without passaging prior to transfection or, alternatively, can be passaged in culture prior to transfection but not immortalized. The primary eukaryotic cells can be primary mammalian cells, such as primary human, non-human primate, mouse, rat, dog, porcine, or rabbit cells. In specific embodiments, the primary cells are primary human cells. In certain embodiments, the cells are human primary endothelial cells, human primary epithelial cells, primary human fibroblasts, primary human smooth muscle cells, or primary human lymphocyte cells. In specific embodiments, the primary cells are primary human T cells.

In certain embodiments of the methods, the DNA template can comprise a nucleic acid sequence encoding an exogenous sequence of interest that is expressed in the transfected primary eukaryotic cells. In some embodiments, the exogenous sequence of interest encodes a chimeric antigen receptor and/or an exogenous T cell receptor. In particular embodiments, the exogenous sequence of interest can encode an engineered endonuclease. In different embodiments, the engineered endonuclease can be an engineered meganuclease, a TALEN, a compact TALEN, a zinc finger nuclease (ZFN), a CRISPR/Cas, or a mega-TAL. In a specific embodiment of the method, the engineered endonuclease is an engineered meganuclease.

In some embodiments of the methods, IRF3 signaling is reduced by introducing an IRF3 signaling inhibitor followed by introducing a DNA template into a primary eukaryotic cell. In other embodiments of the methods, an IRF3 signaling inhibitor is introduced simultaneously with a DNA template into a primary eukaryotic cell. In further embodiments, an IRF3 signaling inhibitor is introduced after the introduction of a DNA template into a primary eukaryotic cell.

In some embodiments of the methods, IRF3 signaling can be reduced by small molecule inhibition of IRF3. In some embodiments, IRF3 signaling is reduced by small molecule inhibition of at least one upstream regulator of IRF3. In particular embodiments, IRF3 signaling is reduced by small molecule inhibition of stimulator of interferon genes (STING), TANK-binding kinase 1 (TBK1), and/or Cyclic GMP-AMP synthase (cGAS). In particular embodiments, IRF3 signaling is reduced by small molecule inhibition of STING. In some embodiments, IRF3 signaling is reduced by small molecule inhibition of cGAS. In some embodiments, IRF3 signaling is reduced by small molecule inhibition of TBK1.

In some embodiments of the methods, IRF3 signaling is reduced by downregulating IRF3 protein expression and/or activity. In certain embodiments, IRF3 signaling is reduced by reducing protein expression and/or activity of least one upstream regulator of IRF3, including STING, TBK1, and/or cGAS. In particular embodiments, IRF3 signaling is reduced by reducing STING protein expression and/or activity. In some embodiments, IRF3 signaling is reduced by reducing cGAS protein expression and/or activity. In some embodiments, IRF3 signaling is reduced by reducing TBK1 protein expression and/or activity.

In certain embodiments of the methods, protein expression and/or activity of IRF3, STING, TBK1, and/or cGAS is reduced by RNA interference (siRNA or shRNA), antisense RNA, site-directed mutagenesis, restriction enzyme digestion followed by re-ligation, PCR-based mutagenesis techniques, allelic exchange, allelic replacement, RNA interference, post-translational modification, or any combination thereof. In specific embodiments, protein expression and/or activity of IRF3, STING, TBK1, and/or cGAS is reduced by RNA interference.

In certain embodiments of the methods, the nucleic acid encoding the endonuclease, or the endonuclease protein, is introduced into the primary eukaryotic cells prior to, simultaneously with, or after introduction of the DNA template. In different embodiments of the methods, the nucleic acid encoding the endonuclease can be an mRNA or a DNA template.

In particular embodiments of the methods, the endonuclease is an engineered endonuclease, a homing endonuclease, or a restriction endonuclease. In particular embodiments, the endonuclease is an engineered endonuclease, such as an engineered meganuclease, a TALEN, a compact TALEN, a zinc finger nuclease (ZFN), a CRISPR/Cas, or a mega-TAL. In certain embodiments, the engineered endonuclease is an engineered meganuclease. In some embodiments, the engineered endonuclease recognizes and cleaves a recognition sequence located within an endogenous gene of interest to produce a cleavage site, and the cleavage site is repaired by non-homologous end joining (NHEJ), wherein expression of the endogenous gene of interest is disrupted and/or activity of a polypeptide encoded by the gene of interest is reduced. In certain embodiments, the total number of cells exhibiting disruption of the gene of interest and/or reduction of the encoded polypeptide is increased when compared to control cells in which IRF3 signaling is not reduced. In particular embodiments, the gene editing efficiency (i.e., the percentage of cells comprising the edited gene) is increased when comprising to controls cells in which IRF3 signaling is not reduced.

In particular embodiments of the methods, the nucleic acid sequence encoding an exogenous sequence of interest lacks substantial homology to the recognition sequence and is inserted at the cleavage site by NHEJ.

In certain embodiments of the methods, the exogenous sequence of interest further comprises sequences homologous to sequences flanking the recognition sequence of the engineered endonuclease, such that the exogenous sequence of interest is inserted at the cleavage site by homologous recombination (i.e., targeted insertion). In some such embodiments, the number of transfected primary eukaryotic cells comprising the exogenous sequence of interest inserted at the cleavage site is increased when compared to control cells in which IRF3 signaling is not reduced. In additional embodiments, the insertion frequency (i.e., the percentage of cells comprising the exogenous sequence of interest inserted at the cleavage site) is increased when compared to control cells in which IRF3 signaling is not reduced.

In specific embodiments of the methods, IRF3 signaling is reduced by reducing IRF3 protein expression and/or activity using RNA interference, the nucleic acid encoding an engineered nuclease is an mRNA encoding an engineered meganuclease, the exogenous sequence of interest encodes a chimeric antigen receptor and is inserted at the cleavage site by homologous recombination, and the primary eukaryotic cells are primary human T cells.

In another specific embodiment of the methods, IRF3 signaling is reduced by reducing STING protein expression and/or activity using RNA interference, the nucleic acid encoding an engineered nuclease is an mRNA encoding an engineered meganuclease, the exogenous gene of interest encodes a chimeric antigen receptor and is inserted at the cleavage site by homologous recombination, and the primary eukaryotic cells are primary human T cells.

In another aspect, the invention provides a method for high throughput screening of primary human T cells expressing a chimeric antigen receptor (CAR) or exogenous T cell receptor (TCR), the method comprising: (a) reducing interferon regulatory factor 3 (IRF3) signaling in the primary human T cells; (b) transfecting a DNA template into the primary human T cells, wherein the DNA template comprises an exogenous nucleic acid sequence encoding a CAR or an exogenous TCR; (c) introducing an endonuclease, or a nucleic acid encoding an endonuclease, into the primary human T cells, wherein the endonuclease recognizes and cleaves a recognition sequence present in the genome of the primary human T cells to produce a cleavage site, and wherein the exogenous nucleic acid sequence is inserted at the cleavage site, and wherein the CAR or the exogenous TCR is expressed on the cell surface; and (d) characterizing a phenotype of the primary human T cells expressing the CAR or the exogenous TCR.

In some embodiments of the method, the DNA template is a single-stranded DNA template or a double-stranded DNA template. In certain embodiments of the method, the DNA template is a plasmid DNA template. In particular embodiments of the method, the DNA template is a linearized DNA template.

In some embodiments of the method, IRF3 signaling is reduced by downregulating protein expression and/or activity of least one upstream regulator of IRF3. In some such embodiments of the method, IRF3 signaling is reduced by downregulating protein expression and/or activity of STING, TBK1, and/or cGAS. In particular embodiments of the method, IRF3 signaling is reduced by downregulating STING protein expression and/or activity. In particular embodiments of the method, IRF3 signaling is reduced by downregulating cGAS protein expression and/or activity. In particular embodiments of the method, IRF3 signaling is reduced by downregulating TBK1 protein expression and/or activity. In other embodiments of the method, IRF3 signaling is reduced by downregulating IRF3 protein expression and/or activity.

In certain embodiments of the method, protein expression and/or activity is downregulated by: (a) RNA interference (siRNA or shRNA); (b) antisense RNA; (c) gene knockout; or (d) any combination thereof. In particular embodiments of the method, protein expression and/or activity is downregulated by RNA interference.

In some embodiments of the method, IRF3 signaling is reduced by small molecule inhibition of at least one upstream regulator of IRF3. In some such embodiments of the method, IRF3 signaling is reduced by small molecule inhibition of stimulator of interferon genes (STING), TANK-binding kinase 1 (TBK1), and/or Cyclic GMP-AMP synthase (cGAS). In particular embodiments of the method, IRF3 signaling is reduced by small molecule inhibition of STING. In particular embodiments of the method, IRF3 signaling is reduced by small molecule inhibition of cGAS. In particular embodiments of the method, IRF3 signaling is reduced by small molecule inhibition of TBK1. In other embodiments of the method, IRF3 signaling is reduced by small molecule inhibition of IRF3.

In some embodiments of the method, the nucleic acid encoding the endonuclease, or the endonuclease protein, is introduced into the primary eukaryotic cells prior to, simultaneously with, or after transfection with the DNA template.

In certain embodiments of the method, the nucleic acid encoding the endonuclease is an mRNA or a DNA template.

In some embodiments of the method, the endonuclease is an engineered endonuclease. In certain embodiments of the method, the engineered endonuclease is an engineered meganuclease, a TALEN, a zinc finger nuclease (ZFN), a CRISPR/Cas, or a mega-TAL. In particular embodiments of the method, the engineered endonuclease is an engineered meganuclease.

In certain embodiments of the method, the recognition sequence is located within a gene of interest, and wherein expression of the gene of interest is disrupted and/or activity of a polypeptide encoded by the gene of interest is reduced.

In another aspect, the invention provides a method for high throughput screening of primary human T cells expressing a chimeric antigen receptor (CAR) or exogenous T cell receptor (TCR), the method comprising: (a) reducing STING signaling in the primary human T cells; (b) transfecting a DNA template into the primary human T cells, wherein the DNA template comprises an exogenous nucleic acid sequence encoding a CAR or an exogenous TCR; (c) introducing an endonuclease, or a nucleic acid encoding an endonuclease, into the primary human T cells, wherein the endonuclease recognizes and cleaves a recognition sequence present in the genome of the primary human T cells to produce a cleavage site, and wherein the exogenous nucleic acid sequence is inserted at the cleavage site, and wherein the CAR or the exogenous TCR is expressed on the cell surface; and (d) characterizing a phenotype of the primary human T cells expressing the CAR or the exogenous TCR.

In some embodiments of the method, the DNA template is a single-stranded DNA template or a double-stranded DNA template. In certain embodiments of the method, the DNA template is a plasmid DNA template. In particular embodiments of the method, the DNA template is a linearized DNA template.

In some embodiments of the method, STING signaling is reduced by downregulating STING protein expression and/or activity. In particular embodiments of the method, STING protein expression and/or activity is downregulated by: (a) RNA interference (siRNA or shRNA); (b) antisense RNA; (c) gene knockout; or (d) any combination thereof. In certain embodiments of the method, STING protein expression and/or activity is downregulated by RNA interference. In other embodiments of the method, STING signaling is reduced by small molecule inhibition.

In some embodiments of the method, the nucleic acid encoding the endonuclease, or the endonuclease protein, is introduced into the primary eukaryotic cells prior to, simultaneously with, or after transfection with the DNA template.

In certain embodiments of the method, the nucleic acid encoding the endonuclease is an mRNA or a DNA template.

In some embodiments of the method, the endonuclease is an engineered endonuclease. In certain embodiments of the method, the engineered endonuclease is an engineered meganuclease, a TALEN, a zinc finger nuclease (ZFN), a CRISPR/Cas, or a mega-TAL. In particular embodiments of the method, the engineered endonuclease is an engineered meganuclease.

In certain embodiments of the method, the recognition sequence is located within a gene of interest, and wherein expression of the gene of interest is disrupted and/or activity of a polypeptide encoded by the gene of interest is reduced.

In some embodiments of the method, the step of characterizing a phenotype comprises determining the frequency of cell surface expression of the CAR or the exogenous TCR.

In some embodiments of the method, the step of characterizing a phenotype comprises determining the memory phenotype of the primary human T cells expressing a CAR or an exogenous TCR.

In some embodiments of the method, the step of characterizing a phenotype comprises determining the CD4⁺ to CD8⁺ ratio of the primary human T cells expressing a CAR or an exogenous TCR.

In some embodiments of the high throughput methods of the invention, the step of characterizing a phenotype comprises quantifying exhaustion markers expressed by the primary human T cells expressing a CAR or an exogenous TCR. In some such embodiments of the method, the exhaustion markers can include, but are not limited to, TIM-3, PD-1, and/or LAG-3.

In some embodiments of the high throughput methods of the invention, the step of characterizing a phenotype comprises quantifying antigen-independent and/or antigen-induced secretion of cytokines by the primary human T cells expressing a CAR or an exogenous TCR. In some such embodiments of the method, the cytokines can include, but are not limited to, interferon-gamma, interleukin-2, tumor necrosis factor alpha, granzyme B, and perforin.

In some embodiments of the high throughput methods of the invention, the step of characterizing a phenotype comprises determining antigen-independent phosphorylated protein expression by the primary human T cells expressing a CAR or an exogenous TCR. In some such embodiments of the method, the phosphorylated protein can include, but is not limited to, CD3ζ, extracellular signal-regulated kinase (ERK), RAC-alpha serine/threonine-protein kinase (AKT1), and p38.

In some embodiments of the high throughput methods of the invention, the step of characterizing a phenotype comprises evaluating cytotoxicity of the primary human T cells expressing a CAR or an exogenous TCR against antigen-bearing target cells.

In some embodiments of the high throughput methods of the invention, the step of characterizing a phenotype comprises determining antigen-independent and/or antigen-induced proliferative capacity of the primary human T cells expressing a CAR or an exogenous TCR.

In some embodiments of the high throughput methods of the invention, the step of characterizing a phenotype comprises evaluating exhaustion of the primary human T cells expressing a CAR or an exogenous TCR after repeated antigen encounter.

In another aspect, the invention provides a genetically-modified primary eukaryotic cell prepared by the any of the methods of described herein. In some embodiments, the cell is a genetically-modified primary human T cell. In particular embodiments, the cell expresses a chimeric antigen receptor or an exogenous T cell receptor.

In another aspect, the invention provides a genetically-modified primary eukaryotic cell comprising a nucleic acid sequence encoding a chimeric antigen receptor or an exogenous T cell receptor, wherein IRF3 signaling is reduced in the cell when compared to a control cell.

In certain embodiments, IRF3 protein expression and/or activity is reduced in the cell when compared to a control cell. In certain embodiments, STING protein expression and/or activity is reduced in the cell when compared to a control cell. In certain embodiments, TBK1 protein expression and/or activity is reduced in the cell when compared to a control cell. In certain embodiments, cGAS protein expression and/or activity is reduced in the cell when compared to a control cell.

In some embodiments, the cell is a genetically-modified primary human T cell.

In another aspect, the invention provides a genetically-modified primary eukaryotic cell comprising a nucleic acid sequence encoding a chimeric antigen receptor or an exogenous T cell receptor, wherein STING signaling is reduced in the cell when compared to a control cell.

In certain embodiments, STING protein expression and/or activity is reduced in the cell when compared to a control cell.

In some embodiments, the cell is a genetically-modified primary human T cell.

### BRIEF DESCRIPTION OF THE FIGURES

FIG. 1. Diagram of the cGAS-STING-TBK1-IRF3 intracellular signaling pathway in response to the introduction of double-stranded DNA into the cytoplasm.

FIGS. 2A and 2B. Inhibition of IRF3 signaling reduces cytotoxicity of linear and plasmid DNA. Primary human T cells were transfected to introduce (i) mRNA encoding an engineered meganuclease (TRC 1-2x.87 EE; the “TRC nuclease”), which recognizes and cleaves a recognition sequence within the T cell receptor alpha constant region (TRAC) gene, (ii) a linear or a plasmid DNA construct that comprises a coding sequence for an anti-CD19 chimeric antigen receptor (CAR) which includes a CD34 epitope, wherein the CAR coding sequence further comprises homology arms that are homologous to sequences upstream and downstream of the TRC nuclease cleavage site, and/or (iii) an siRNA smartpool or individual siRNAs directed to IRF3. Total cell number was determined at day 3 and day 5 post-transfection to determine the effect of IRF3 knockdown on DNA-induced cytotoxicity. FIG. 2A shows transfection with linear DNA template. FIG. 2B shows transfection with plasmid DNA template.

FIGS. 3A and 3B. Inhibition of IRF3 signaling via STING knockdown reduces cytotoxicity of linear and plasmid DNA. Primary human T cells were transfected to introduce (i) mRNA encoding the TRC nuclease, (ii) a linear or a plasmid DNA construct that comprises a coding sequence for the anti-CD19 CAR, and/or (iii) an siRNA smartpool or individual siRNAs directed to STING. Total cell number was determined at day 3 and day 5 post-transfection to determine the effect of STING knockdown on DNA-induced cytotoxicity. FIG. 3A shows transfection with linear DNA template. FIG. 3B shows transfection with plasmid DNA template.

FIGS. 4A, 4B, 4C, and 4D. Effect of IRF3 inhibition on gene editing and targeted gene insertion in primary human T cells transfected with a linear DNA construct. FIG. 4A shows total number of edited CD3− cells. FIG. 4B shows percentage of edited CD3− cells in treated populations. FIG. 4C shows total number of CD3−/CAR+ cells. FIG. 4D shows percentage of CD3−/CAR+ cells in treatment populations.

FIGS. 5A, 5B, 5C, and 5D. Effect of IRF3 inhibition on gene editing and targeted gene insertion in primary human T cells transfected with a linear DNA construct. FIG. 5A shows total number of edited CD3− cells. FIG. 5B shows percentage of edited CD3− cells in treated populations. FIG. 5C shows total number of CD3−/CAR+ cells. FIG. 5D shows percentage of CD3−/CAR+ cells in treatment populations.

FIGS. 6A, 6B, 6C, and 6D. Effect of IRF3 inhibition on gene editing and targeted gene insertion in primary human T cells transfected with a plasmid DNA construct. FIG. 6A shows total number of edited CD3− cells. FIG. 6B shows percentage of edited CD3− cells in treated populations. FIG. 6C shows total number of CD3−/CAR+ cells. FIG. 6D shows percentage of CD3−/CAR+ cells in treatment populations.

FIGS. 7A, 7B, 7C, and 7D. Effect of IRF3 inhibition on gene editing and targeted gene insertion in primary human T cells transfected with a plasmid DNA construct. FIG. 7A shows total number of edited CD3− cells. FIG. 7B shows percentage of edited CD3− cells in treated populations. FIG. 7C shows total number of CD3−/CAR+ cells. FIG. 7D shows percentage of CD3−/CAR+ cells in treatment populations.

FIGS. 8A and 8B. Effect of STING inhibition on gene editing and targeted gene insertion in primary human T cells transfected with a linear DNA construct. FIG. 8A shows total number of edited CD3− cells. FIG. 8B shows total number of CD3−/CAR+ cells.

FIGS. 9A, 9B, 9C, and 9D. Effect of STING inhibition on gene editing and targeted gene insertion in primary human T cells transfected with a linear DNA construct. FIG. 9A shows total number of edited CD3− cells. FIG. 9B shows percentage of edited CD3− cells in treated populations. FIG. 9C shows total number of CD3−/CAR+ cells. FIG. 9D shows percentage of CD3−/CAR+ cells in treatment populations.

FIGS. 10A, 10B, 10C, and 10D. Effect of STING inhibition on gene editing and targeted gene insertion in primary human T cells transfected with a plasmid DNA construct. FIG. 10A shows total number of edited CD3− cells. FIG. 10B shows percentage of edited CD3− cells in treated populations. FIG. 10C shows total number of CD3−/CAR+ cells. FIG. 10D shows percentage of CD3−/CAR+ cells in treatment populations.

FIGS. 11A, 11B, 11C, and 11D. Effect of STING inhibition on gene editing and targeted gene insertion in primary human T cells transfected with a plasmid DNA construct. FIG. 11A shows total number of edited CD3− cells. FIG. 11B shows percentage of edited CD3− cells in treated populations. FIG. 11C shows total number of CD3−/CAR+ cells. FIG. 11D shows percentage of CD3−/CAR+ cells in treatment populations.

FIGS. 12A, 12B, 12C, 12D, and 12E. Effect of IRF3 or STING inhibition on primary human T cell phenotype. FIG. 12A shows mock treated cells. FIG. 12B shows cells transfected to introduce mRNA encoding the TRC nuclease. FIG. 12C shows cells transfected to introduce mRNA encoding the TRC nuclease and a linear DNA template. FIG. 12D shows cells transfected to introduce mRNA encoding the TRC nuclease, a linear DNA template, and an IRF3 siRNA smartpool. FIG. 12E shows cells transfected to introduce mRNA encoding the TRC nuclease, a linear DNA template, and the STING-1 siRNA.

FIGS. 13A, 13B, 13C, 13D, and 13E. Effect of IRF3 or STING inhibition on the distribution of CD4 and CD8 cells in a population of primary human T cells. FIG. 13A shows mock treated cells. FIG. 13B shows cells transfected to introduce mRNA encoding the TRC nuclease. FIG. 13C shows cells transfected to introduce mRNA encoding the TRC nuclease and a linear DNA template. FIG. 13D shows cells transfected to introduce mRNA encoding the TRC nuclease, a linear DNA template, and an IRF3 siRNA smartpool. FIG. 13E shows cells transfected to introduce mRNA encoding the TRC nuclease, a linear DNA template, and the STING-1 siRNA.

FIGS. 14A and 14B. STING knockdown by siRNA. Western blot analysis of STING protein expression over time in primary human T cells following introduction of a STING smartpool of siRNAs. FIG. 14 A shows Western blot analysis of STING protein. B) β-actin control.

### BRIEF DESCRIPTION OF THE SEQUENCES

SEQ ID NO: 1 sets forth the LAGLIDADG motif.

SEQ ID NO: 2 sets forth the nucleic acid sequence of the IRF3 siRNA-1.

SEQ ID NO: 3 sets forth the nucleic acid sequence of the IRF3 siRNA-2.

SEQ ID NO: 4 sets forth the nucleic acid sequence of the IRF3 siRNA-3.

SEQ ID NO: 5 sets forth the nucleic acid sequence of the IRF3 siRNA-4.

SEQ ID NO: 6 sets forth the nucleic acid sequence of the STING siRNA-1.

SEQ ID NO: 7 sets forth the nucleic acid sequence of the STING siRNA-2.

SEQ ID NO: 8 sets forth the nucleic acid sequence of the STING siRNA-3.

SEQ ID NO: 9 sets forth the nucleic acid sequence of the STING siRNA-4.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the terms “IRF3” or “interferon regulatory factor 3” refer to a transcription factor encoded by the human IRF3 gene (NCBI Gene No. 3661) or a variant of the human IRF3 gene which still encodes a functional IRF3 protein. These terms also refer to analogous IRF3 proteins and genes present in other species. “IRF3 signaling” generally refers to intracellular signaling that results in dimerization and activation of IRF3, translocation to the nucleus, and subsequent activation of type I interferons and inflammatory cytokines in a cell.

As used herein, the terms “cGAS” or “cyclic GMAP-AMP synthase” refer to a cytosolic DNA sensing polypeptide encoded by the human MB21D1 gene (NCBI Gene No. 115004) or a variant of the MB21D1 gene which still encodes a functional cGAS protein. These terms also refer to analogous cGAS proteins and genes present in other species.

As used herein, the terms “STING” or “stimulator of interferon genes” refer to a protein encoded by the human TMEM173 gene (NCBI Gene No. 340061) or a variant of the human TMEM173 gene which still encodes a functional STING protein. These terms also refer to analogous STING proteins and genes present in other species.

As used herein, the terms “TBK1” or “TANK binding kinase 1” refer to a protein encoded by the human TBK1 gene (NCBI Gene No. 29110) or a variant of the human TBK1 gene which still encodes a functional TBK1 protein. These terms also refer to analogous TBK1 proteins and genes present in other species.

As used herein, the term “DNA template” refers to an artificially constructed (i.e., recombinant) segment of nucleic acid preferably comprising a sequence of interest, such as a coding sequence for a gene of interest. In the present invention, a DNA template can be a double-stranded DNA template or, alternatively, a single-stranded DNA template, which is introduced into a primary eukaryotic cell. The DNA template used in the invention can be a plasmid, or circularized, DNA template. Alternatively, the DNA template can be a linearized DNA template, which can be prepared, for example, by linearizing a plasmid DNA using a restriction enzyme.

As used herein, the term “gene editing efficiency” refers to the ability of a nuclease, such as an engineered endonuclease, to produce an insertion or a deletion (i.e., an indel) in the genome of a percentage of cells in a population of primary eukaryotic cells. For example, under conditions where gene editing efficiency is 50%, this means that 50% of the cells in the treated population comprise a detectable indel.

As used herein, the term “targeted insertion” refers to the insertion of an exogenous sequence of interest into a targeted site in the genome of a cell. Insertion can be the result of homologous recombination or homology directed repair (HDR) at a double-strand break in the genome (e.g., resulting from cleavage by an endonuclease), which can be promoted by the inclusion of homology arms flanking the exogenous sequence of interest, wherein the homology arms have homology to regions upstream and downstream of the targeted site.

As used herein, the term “insertion frequency” refers to the percentage of cells in a population of primary eukaryotic cells in which an exogenous sequence of interest is inserted into a targeted site in the genome (e.g., an endonuclease cleavage site) by homologous recombination or HDR. For example, under conditions where insertion frequency is 50%, this means that 50% of the cells in the treated population comprise the exogenous sequence of interest inserted into the genome at the targeted site.

As used herein, the term “reduced” refers to any reduction in the symptoms or severity of a disease, any reduction in the proliferation or number of cancerous cells, or any reduction in protein expression, protein activity, or IRF3 signaling. In any situation, such a reduction may be up to 5%, 10%, 20%, 30%, 40%, 50%, 60%, 70%, 80%, 90%, 95%, or up to 100%, when compared to appropriate control cells.

As used herein, “cytotoxicity” refers to the death of a primary eukaryotic cell following introduction of a DNA template. Likewise, as used herein “reduced cytotoxicity” refers a reduction in the cytotoxicity of primary eukaryotic cells. Reduction of cytotoxicity can be up to or at least 5%, 10%, 20%, 30%, 40%, 50%, 60%, 70%, 80%, 90%, 95%, or up to 100% when compared to appropriate control cells. Reduction of cytotoxicity can be 1-5%, 2-10%, 5-15%, 10-20%, 15-30%, 20-50%, 30-60%, 50-75%, or 75-100% reduction when compared to appropriate control cells. Cytotoxicity can be determined, for example, by measuring total viable cell number at various time points after DNA transfection.

As used herein, the term “increased” refers to any increase in the survival, increase in total number of cells comprising a gene edit, increase in gene editing efficiency, increase in the total number of cells comprising a targeted insertion of an exogenous sequence of interest, or increase in insertion frequency of primary eukaryotic cells modified to reduce IRF3 signaling, as disclosed herein. Such an increase may be up to 5%, 10%, 20%, 30%, 40%, 50%, 60%, 70%, 80%, 90%, 95%, or up to 100% when compared to appropriate control cells. Such an increase may also be up to 2-fold, 3-fold, 4-fold, 5-fold, 10-fold, 20-fold, 50-fold, 100-fold, or more, when compared to appropriate control cells. Any methods known in the art can be used to determine changes in cell viability, the occurrence of gene editing at a cleavage site, and targeted insertion of an exogenous sequence of interest into the genome at a cleavage site.

Accordingly, the term “reduced” encompasses both a partial reduction and a complete reduction of a disease state, protein expression, protein activity, or IRF3 signaling.

The terms “recombinant DNA construct,” “recombinant construct,” “expression cassette,” “expression construct,” “chimeric construct,” “construct,” and “recombinant DNA fragment” are used interchangeably herein and are nucleic acid fragments. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in that art suitable for delivering a gene encoding a meganuclease of the invention to a target cell. The skilled artisan is well aware of the genetic elements that must be present on the vector in order to successfully transform, select and propagate host cells comprising any of the isolated nucleotides or nucleic acid sequences of the invention.

As used herein, a “vector” can also refer to a viral vector. Viral vectors can include, without limitation, retroviral vectors, lentiviral vectors, adenoviral vectors, and adeno-associated viral vectors (AAV).

As used herein, a “polycistronic” mRNA refers to a single messenger RNA which comprises two or more coding sequences (i.e., cistrons) and encodes more than one protein. A polycistronic mRNA can comprise any element known in the art to allow for the translation of two or more genes from the same mRNA molecule including, but not limited to, an IRES element, a T2A element, a P2A element, an E2A element, and an F2A element.

As used herein an “exogenous sequence of interest” or “exogenous gene of interest” is not limited in any way and may be any nucleic acid that is desired to be delivered to, integrated, transcribed, translated, and/or expressed in a primary eukaryotic cell. The exogenous sequence of interest may encode a receptor such as a chimeric antigen receptor or an exogenous T cell receptor, or may encode a functional product, such as a protein or an RNA molecule. The exogenous sequence of interest can also encode an engineered meganuclease or other DNA endonuclease. The exogenous sequence of interest can also encode an antigen or antibody. The exogenous sequence of interest is generally operatively linked to other sequences that are useful for obtaining the desired expression of the sequence of interest, such as transcriptional regulatory sequences.

As used herein, a “primary cell” or “primary eukaryotic cell” refers to a cell or cell line that has been taken directly from an individual. Primary cells include isolated cells that have not been passaged in culture, isolated cells that have been passaged and maintained under cell culture conditions without immortalization, and cells that have been immortalized and can be maintained under cell culture conditions indefinitely.

As used herein, a “human T cell” or “T cell” refers to a T cell isolated from a human donor. Human T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); (c) a cell genetically identical to the genetically-modified cell but which is not exposed to conditions or stimuli or further genetic modifications that would induce expression of altered genotype or phenotype; (d) a cell having wild-type IRF3 signaling; or (e) a cell into which an IRF3 signaling inhibitor has not been introduced. Likewise, a “control cell population” refers to a population of control cells.

As used herein, the terms “nuclease” and “endonuclease” are used interchangeably to refer to naturally-occurring or engineered enzymes which cleave a phosphodiester bond within a polynucleotide chain.

As used herein, the term “meganuclease” refers to an endonuclease that binds double-stranded DNA at a recognition sequence that is greater than 12 base pairs. Preferably, the recognition sequence for a meganuclease of the invention is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859). A meganuclease as used herein binds to double-stranded DNA as a heterodimer. A meganuclease may also be a “single-chain meganuclease” in which a pair of DNA-binding domains are joined into a single polypeptide using a peptide linker. The term “homing endonuclease” is synonymous with the term “meganuclease.” Meganucleases of the invention are substantially non-toxic when expressed in cells without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two meganuclease subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251 and 9,434,931.

As used herein, the term “TALEN” refers to an endonuclease comprising a DNA-binding domain comprising 16-22 TAL domain repeats fused to any portion of the FokI nuclease domain.

As used herein, the term “Compact TALEN” refers to an endonuclease comprising a DNA-binding domain with 16-22 TAL domain repeats fused in any orientation to any portion of the I-TevI homing endonuclease.

As used herein, the term “zinc finger nuclease” or “ZFN” refers to a chimeric endonuclease comprising a zinc finger DNA-binding domain fused to the nuclease domain of the FokI restriction enzyme. The zinc finger domain can be redesigned through rational or experimental means to produce a protein which binds to a pre-determined DNA sequence ˜18 basepairs in length, comprising a pair of nine basepair half-sites separated by 2-10 basepairs. Cleavage by a zinc finger nuclease can create a blunt end or a 5′ overhand of variable length (frequently four basepairs).

As used herein, the term “CRISPR” refers to a caspase-based endonuclease comprising a caspase, such as Cas9, Cpf1, or any other suitable caspase, and a guide RNA that directs DNA cleavage of the caspase by hybridizing to a recognition site in the genomic DNA.

As used herein, the term “megaTAL” refers to a single-chain endonuclease comprising a transcription activator-like effector (TALE) DNA binding domain with an engineered, sequence-specific homing endonuclease.

As used herein, with respect to a protein, the term “recombinant” or “engineered” means having an altered amino acid sequence as a result of the application of genetic engineering techniques to nucleic acids which encode the protein, and cells or organisms which express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the term “recognition sequence” refers to a DNA sequence that is bound and cleaved by an endonuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ “overhangs”. “Overhangs”, or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a Compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a Compact TALEN produces two basepair 3′ overhangs. In the case of a CRISPR, the recognition sequence is the sequence, typically 16-24 basepairs, to which the guide RNA binds to direct cleavage by Cas9, Cpf1, or another suitable caspase. Cleavage by a CRISPR produces blunt ends. In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “target site” or “target sequence” refers to a region of the chromosomal DNA of a cell comprising a recognition sequence for a nuclease.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered endonucleases can be used to effectively knock-out a gene in a population of cells.

As used herein with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first proteins corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first proteins corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1, or 2 if the variable is inherently discrete, and can take the values 0.0, 0.1, 0.01, 0.001, or any other real values ≥0 and ≤2 if the variable is inherently continuous.

### 2.1 Principle of the Invention

An obstacle in the use and modification of many primary cells is the significant cytotoxicity observed following transfection with circular or linearized DNA. The inventors have successfully reduced DNA-induced cytotoxicity by inhibiting intracellular signaling through the Interferon Regulatory Factor 3 (IRF3) signaling pathway. The inventors have also observed that inhibition of IRF3 signaling can increase the number of gene-edited cells, and increase gene editing efficiency, when an engineered nuclease is introduced into primary eukaryotic cells along with a DNA template. The inventors have further observed that inhibition of IRF3 signaling can increase the number of primary eukaryotic cells comprising a targeted insertion of an exogenous sequence into their genome, and can increase insertion frequency, when an engineered nuclease is introduced into primary eukaryotic cells along with a DNA template.

Consequently, in some aspects, the methods of the invention can be utilized as a high throughput method for screening human T cells that express a chimeric antigen receptor (CAR) or an exogenous T cell receptor (TCR). Due to the toxicity associated with primary cell transfection with DNA, one typically has to generate an AAV in order to deliver a donor template into a human T cell for insertion of a CAR or exogenous TCR coding sequence into the genome. Generation of AAVs can be extremely costly, time-consuming, and labor-intensive, particularly when numerous constructs, and variations of constructs, need to be tested. By contrast, the methods of the invention allow for a rapid, low-cost, simple evaluation of CAR and exogenous TCR constructs expressed by human T cells without having to generate AAV constructs.

The observed changes are not dependent on the method of inhibition of IRF3 signaling. Rather, inhibition of IRF3 signaling can be achieved in a number of ways, and any method can be used to inhibit any step in the IRF3 signaling pathway. For example, methods for inhibition can include small molecule inhibition of IRF3 directly, and/or small molecule inhibition of upstream effectors of IRF3, such as TANK-binding kinase 1 (TBK1), stimulator of interferon genes (STING), and/or Cyclic GMP-AMP synthase (cGAS). Inhibition of IRF3 signaling can also be achieved by downregulation of IRF3 protein expression, and/or downregulation of protein expression of upstream effectors of IRF3, including TBK1, STING, and/or cGAS.

The methods of the invention result in a greater number of genetically-modified primary eukaryotic cells available for further modification or research use. For example, the methods of the invention can be utilized to express a gene of interest in primary eukaryotic cells. In a particular example, inhibition of IRF3 can be used to produce CAR T cells (i.e., primary human T cells expressing a chimeric antigen receptor) by introducing the CAR coding sequence via a transfected DNA template, rather than by viral transduction.

### 2.2 Reduction of IRF3 Signaling

The IRF3 pathway relies on the activation of cGAS, STING, TBK1, and IRF3 in order to recognize foreign cytoplasmic DNA and activate the immune system to effectuate removal of the invading nucleic acid. As used herein, “IRF3 signaling” refers collectively to the expression and activity of cGAS, STING, TBK1, and IRF3, which results in entry of phosphorylated IRF3 into the nucleus of a primary eukaryotic cell and subsequent downstream activation of type I interferons and/or inflammatory cytokines. According to the invention, IRF3 signaling in a primary eukaryotic cell can be reduced by inhibiting the expression and/or activity of IRF3 directly, or by inhibiting the expression and/or activity of any of the upstream effectors of IRF3, including cGAS, STING, and TBK1.

In the context of the invention, an “IRF3 signaling inhibitor” or “IRF3 inhibitor” is any molecule that reduces IRF3 signaling in a primary eukaryotic cell. An IRF3 signaling inhibitor may act directly to inhibit the activation and/or expression of IRF3, or it may act to inhibit the activation and/or expression of cGAS, STING, or TBK1. An IRF3 signaling inhibitor can be a molecule that, itself, inhibits IRF3 signaling, or it may encode a molecule that inhibits IRF3 signaling in the cell.

### 2.2a Small Molecule Inhibition and Other Means of Inhibiting Activity

In certain embodiments of the invention, IRF3 signaling is reduced in primary eukaryotic cells by using a small molecule inhibitor of IRF3 directly or, alternatively, a small molecule inhibitor of cGAS, STING, and/or TBK1. As used herein, “small molecule inhibitors” include, but are not limited to, small peptides or peptide-like molecules, soluble peptides, and synthetic non-peptidyl organic or inorganic compounds.

IRF3 signaling can also be inhibited by use of non-small molecule inhibitors such as, for example, aptamers, peptides, antibodies, or peptidomimetics.

### 2.2b Downregulation of Protein Expression

In other embodiments of the invention, IRF3 signaling can be reduced by downregulating protein expression of IRF3 directly or, alternatively, downregulating protein expression of cGAS, STING, and/or TBK1.

Protein expression of IRF3, cGAS, STING, and/or TBK1 can be reduced by any method known in the art for reducing protein expression. For example, protein expression can be reduced by using RNA interference such as siRNA or shRNA, by using antisense RNA, or by knocking out a gene encoding cGAS, STING, TBK1 and/or IRF3. Protein expression can also be reduced by gene knockout, for example, by the use of engineered endonucleases.

RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559.

The term “siRNA” as used herein refers to small interfering RNA, also known as short interfering RNA or silencing RNA. siRNAs can be, for example, 18 to 30, 20 to 25, 21 to 23 or 21 nucleotide-long double-stranded RNA molecules. An “shRNA” as used herein is a short hairpin RNA, which is a sequence of RNA that makes a tight hairpin turn that can also be used to silence gene expression via RNA interference. shRNA can by operably linked to the U6 promoter expression. The shRNA hairpin structure is cleaved by the cellular machinery into siRNA. shRNA disclosed herein can comprise a sequence complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, or 23 nucleotides of the mRNA of cGAS, STING, TBK1 and/or IRF3.

Knockdown of genes encoding IRF3, cGAS, STING, and/or TBK1 can be accomplished through various means known in the art. For example, such genes can be targeted by engineered endonucleases which are designed to recognize and cleave a recognition sequence therein. Engineered endonucleases can include those described herein, including engineered meganucleases, TALENs, compact TALENs, CRISPR/Cas, and mega-TALs. Such endonucleases can be engineered to produce a double-strand break within the target gene, which is then repaired by non-homologous end joining (NHEJ). NHEJ often results in the insertion or deletion of nucleotides at the repaired cleavage site, resulting in a mutation of the gene and/or a shift in frame that results in reduced or complete knockout of gene expression.

In addition to engineered endonucleases, any other means in the art can be used to introduce a mutation that inhibits gene expression. The mutation can be an insertion, a deletion, a substitution, or a combination thereof, provided that the mutation leads to a decrease in the expression of IRF3, cGAS, STING, and/or TBK1. In specific embodiments, recombinant DNA technology can be used to introduce a mutation into a specific site on a chromosome. In some embodiments, the insertion or deletion of a single base pair could render a gene encoding IRF3, cGAS, STING, and/or TBK1 non-functional, thereby decreasing protein expression, since as a result of such a mutation, the remaining base pairs are no longer in the correct reading frame. In other embodiments, multiple base pairs are inserted or removed, such as about 2, 5, 10, 20, 30, 40, 50, 75, 100, 150, 200, 300, 400, 500, or more, base pairs. In still other embodiments, the entire length of the gene encoding IRF3, cGAS, STING, and/or TBK1 is deleted. Mutations introducing a stop-codon in the open reading frame, or mutations causing a frame-shift in the open reading frame could be used to reduce the expression of a gene encoding IRF3, cGAS, STING, and/or TBK1.

Other techniques for decreasing the expression of a gene encoding IRF3, cGAS, STING, and/or TBK1, in order to reduce IRF3 signaling, are well-known in the art. For example, techniques may include modification of a gene by site-directed mutagenesis, restriction enzyme digestion followed by re-ligation, PCR-based mutagenesis techniques, allelic exchange, allelic replacement, or post-translational modification. Standard recombinant DNA techniques are all known in the art and described in Maniatis/Sambrook (Sambrook, J. et al. Molecular cloning: a laboratory manual. ISBN 0-87969-309-6). Site-directed mutations can be made by means of in vitro site directed mutagenesis using methods well known in the art.

Changes in protein expression of IRF3, cGAS, STING, and/or TBK1 can be determined by standard methods known in the art. For example, expression of cGAS, STING, TBK1 and/or IRF3 can be determined by detecting or measuring protein levels of IRF3, cGAS, STING, and/or TBK1 in a cell, cell population, or cell lysate. Protein expression of cGAS can be indirectly determined by measuring the synthesis of cGAMP or 2′3′-cGAMP, or by measuring the relocation of STING from the ER to the perinuclear Golgi. Protein expression of STING can be indirectly determined by measuring the activation of TBK1, which is characterized by the formation of large punctate structures. Protein expression of TBK1 can be indirectly determined by measuring the phosphorylation and transcription of IRF3. Protein activity of IRF3 can be indirectly determined by measuring the level of dimerized IRF3 or the activation of type I interferons and inflammatory cytokines.

2.2c Introduction of IRF3 Signaling Inhibitors into Primary Eukaryotic Cells

Inhibitory molecules, such as inhibitory small molecules, nucleic acid molecules (e.g., siRNA or shRNA), ribozymes, peptides, antibodies, antagonists, aptamers, peptidomimetics, and the like, which reduce the expression or activity of IRF3, cGAS, STING, and/or TBK1, can be introduced into primary eukaryotic cells using any method known in the art for introduction of molecules into eukaryotic cells. In this context, “introducing” is intended to mean presenting the molecule to the eukaryotic cell in such a manner that it gains access to the interior of the primary eukaryotic cell. The methods provided herein do not depend on a particular method for introducing IRF3 signaling inhibitors into a primary eukaryotic cell, only that the inhibitors gain access to the interior of the primary eukaryotic cell. Methods for introducing the various IRF3 inhibitors described herein into primary eukaryotic cells are known in the art and include, but are not limited to, stable transformation methods, transient transformation methods, and virus-mediated methods.

Thus, “introduced” in the context of inserting an inhibitory nucleic acid molecule into a primary eukaryotic cell can mean “transfection” or “transformation” or “transduction” and includes reference to the incorporation of a nucleic acid fragment into a eukaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA). The term “transfection” and “transduction” are interchangeable and refer to the process by which an exogenous DNA sequence is introduced into a eukaryotic host cell. Transfection (or transduction) can be achieved by any one of a number of means including electroporation, nucleofection, microinjection, gene gun delivery, retroviral infection, lipofection, superfection and the like.

Inhibitory molecules can be introduced into a primary eukaryotic cell at any stage of primary cell culture desired. For example, inhibitory molecules can be introduced into primary eukaryotic cells that have been maintained in culture without passaging prior to introduction of the inhibitory molecule. In some embodiments, primary eukaryotic cells are passaged in culture prior to introduction of the inhibitory molecule, but the cells are not immortalized.

### 2.3 Primary Eukaryotic Cells

Any primary eukaryotic cell can be used in the methods and compositions disclosed herein. In certain embodiments, the primary eukaryotic cell is a primary mammalian cell. The primary cell can be a primary human, primary non-human primate, primary rat, primary rabbit, primary monkey, primary porcine, primary equine, primary avian, primary bovine, or any other primary eukaryotic cell. In specific embodiments, the primary eukaryotic cells are primary human cells. In certain embodiments, the cells are human primary endothelial cells, primary human epithelial cells, primary human fibroblasts, primary human smooth muscle cells, or primary human lymphocyte cells. In specific embodiments, the primary cells are primary human T cells.

For example, the primary mammalian cell can be an antigen-presenting cell, a dendritic cell, a macrophage, a pluripotent cell, a stem cell, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius' cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, and a zymogenic cell. In specific embodiments, the primary eukaryotic cell is a primary T cell, such as a primary human T cell. In certain embodiments, the cells are primary cells obtained from an individual, which may optionally be returned to the same individual or a different individual following reduction in IRF3 signaling and/or genetic modification.

Primary human T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, T cells can be obtained from a unit of blood collected from a subject using any number of techniques known to the skilled artisan, such as FICOLL separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps.

In another embodiment, T cells can be isolated from peripheral blood lymphocytes by lysing the red blood cells and depleting the monocytes, for example, by centrifugation through a PERCOLL gradient or by counterflow centrifugal elutriation.

As used herein, a “control” or “control cell” refers to a cell that provides a reference point for measuring changes in genotype or phenotype of a genetically-modified cell. For example, in some embodiments, a control cell or control cell population refers to a cell or cell population into which a DNA template has been introduced, but into which no IRF3 inhibitor was introduced. Likewise, a control cell or control cell population can refer to a cell or cell population into which an endonuclease, or polynucleotide encoding an endonuclease, was introduced, but into which no IRF3 inhibitor was introduced.

In specific embodiments, cytotoxicity is reduced, gene editing efficiency is increased, and/or insertion frequency is increased, by introducing an IRF3 signaling inhibitor into a primary eukaryotic cell, followed by introducing a DNA template into the same primary cell. In some embodiments, cytotoxicity is reduced, gene editing efficiency is increased, and/or insertion frequency is increased, by introducing an IRF3 signaling inhibitor into a primary eukaryotic cell, and simultaneously introducing a DNA template into the same primary cell. In other embodiments, cytotoxicity is reduced, gene editing efficiency is increased, and/or insertion frequency is increased, by introducing an IRF3 signaling inhibitor into a primary eukaryotic cell, after introducing a DNA template into the same primary cell.

### 2.4 Introduction of a DNA Template

The methods disclosed herein for reducing IRF3 signaling can increase the total number of surviving primary cells (i.e., reducing cytotoxicity), the total number of gene-edited cells, increase gene editing efficiency, gene-editing efficiency, the total number of cells comprising a targeted insertion of exogenous sequence of interest, and/or insertion frequency following introduction of a DNA template into a primary cell. As used herein, an increase in “survival” refers to an increase in the total number of viable cells following introduction of a DNA template and introduction of an IRF3 signaling inhibitor when compared to the total number of cells following introduction of a DNA template without reducing IRF3 signaling.

In specific embodiments, the total number of viable cells following introduction of a DNA template and introduction of an IRF3 signaling inhibitor increases by 50, 100, 150, 200, 250, 500, 1000, 2000, 3000, 4000, 5000, 7500, 1×10⁴, 1×10⁵, 1×10⁶, 1×10⁷, 1×10⁸, 1×10⁹, 1×10¹⁰ cells, or more, compared to appropriate control cells. Alternatively, the total number of cells following introduction of a DNA template and introduction of an IRF3 signaling inhibitor can increase by 1%, 2%, 3%, 4%, 5%, 6%, 8%, 10%, 12%, 15%, 20%, 25%, 30%, 35%, 40%, 50%, 60%, 70%, 80%, 90%, 100%, 250%, 500%, 1000%, or more, compared to appropriate control cells. Moreover, the total number of cells following introduction of a DNA template and introduction of an IRF3 signaling inhibitor can increase by up to 2 fold, up to 3 fold, up to 4 fold, up to 5 fold, up to 10 fold, up to 20 fold, up to 50 fold, up to 100 fold, up to 500 fold, up to 1000 fold, or more, compared to appropriate control cells. The total number of viable cells can be measured by any means in the art.

In specific embodiments, the total number of cells comprising an edited gene following introduction of a DNA template, an engineered endonuclease, and an IRF3 signaling inhibitor can increase by 50, 100, 150, 200, 250, 500, 1000, 2000, 3000, 4000, 5000, 7500, 1×10⁴, 1×10⁵, 1×10⁶, 1×10⁷, 1×10⁸, 1×10⁹, 1×10¹⁰ cells, or more, compared to appropriate control cells. Alternatively, the total number of cells comprising an edited gene following introduction of a DNA template, an engineered endonuclease, and an IRF3 signaling inhibitor can increase by 1%, 2%, 3%, 4%, 5%, 6%, 8%, 10%, 12%, 15%, 20%, 25%, 30%, 35%, 40%, 50%, 60%, 70%, 80%, 90%, 100%, 250%, 500%, 1000%, or more, compared to appropriate control cells. Moreover, the total number of cells comprising an edited gene following introduction of a DNA template, an engineered endonuclease, and an IRF3 signaling inhibitor can increase by up to 2 fold, up to 3 fold, up to 4 fold, up to 5 fold, up to 10 fold, up to 20 fold, up to 50 fold, up to 100 fold, up to 500 fold, up to 1000 fold, or more, compared to appropriate control cells. The total number of cells comprising an edited gene can be measured by any means in the art.

In specific embodiments, the percentage of cells comprising an edited gene following introduction of a DNA template, an engineered endonuclease, and an IRF3 signaling inhibitor can increase by 1%, 2%, 3%, 4%, 5%, 6%, 8%, 10%, 12%, 15%, 20%, 25%, 30%, 35%, 40%, 50%, 60%, 70%, 80%, 90%, 100%, 250%, 500%, 1000%, or more, compared to appropriate control cells. Moreover, the percentage of cells comprising an edited gene following introduction of a DNA template, an engineered endonuclease, and an IRF3 signaling inhibitor can increase by up to 2 fold, up to 3 fold, up to 4 fold, up to 5 fold, up to 10 fold, up to 20 fold, up to 50 fold, up to 100 fold, up to 500 fold, up to 1000 fold, or more, compared to appropriate control cells. The percentage of cells comprising an edited gene can be measured by any means in the art.

In specific embodiments, the total number of cells comprising an exogenous sequence of interest inserted at a cleavage site following introduction of a DNA template, an engineered endonuclease, and an IRF3 signaling inhibitor can increase by 50, 100, 150, 200, 250, 500, 1000, 2000, 3000, 4000, 5000, 7500, 1×10⁴, 1×10⁵, 1×10⁶, 1×10⁷, 1×10⁸, 1×10⁹, 1×10¹⁰ cells, or more, compared to appropriate control cells. Alternatively, the total number of cells comprising an exogenous sequence of interest inserted at a cleavage site following introduction of a DNA template, an engineered endonuclease, and an IRF3 signaling inhibitor can increase by 1%, 2%, 3%, 4%, 5%, 6%, 8%, 10%, 12%, 15%, 20%, 25%, 30%, 35%, 40%, 50%, 60%, 70%, 80%, 90%, 100%, 250%, 500%, 1000%, or more, compared to appropriate control cells. Moreover, the total number of cells comprising an exogenous sequence of interest inserted at a cleavage site following introduction of a DNA template, an engineered endonuclease, and an IRF3 signaling inhibitor can increase by up to 2 fold, up to 3 fold, up to 4 fold, up to 5 fold, up to 10 fold, up to 20 fold, up to 50 fold, up to 100 fold, up to 500 fold, up to 1000 fold, or more, compared to appropriate control cells. The total number of cells comprising an exogenous sequence of interest inserted at a cleavage site can be measured by any means in the art.

In specific embodiments, the percentage of cells comprising an exogenous sequence of interest inserted at a cleavage site following introduction of a DNA template, an engineered endonuclease, and an IRF3 signaling inhibitor can increase by 1%, 2%, 3%, 4%, 5%, 6%, 8%, 10%, 12%, 15%, 20%, 25%, 30%, 35%, 40%, 50%, 60%, 70%, 80%, 90%, 100%, 250%, 500%, 1000%, or more, compared to appropriate control cells. Moreover, the percentage of cells comprising an exogenous sequence of interest inserted at a cleavage site following introduction of a DNA template, an engineered endonuclease, and an IRF3 signaling inhibitor can increase by up to 2 fold, up to 3 fold, up to 4 fold, up to 5 fold, up to 10 fold, up to 20 fold, up to 50 fold, up to 100 fold, up to 500 fold, up to 1000 fold, or more, compared to appropriate control cells. The percentage of cells comprising an exogenous sequence of interest inserted at a cleavage site can be measured by any means in the art.

In some embodiments, the DNA template can be a single-stranded DNA template or a double-stranded DNA template. The DNA template can encode any exogenous gene of interest or exogenous sequence of interest. In some embodiments the DNA template encodes a chimeric antigen receptor or an exogenous T cell receptor. In particular embodiments, the DNA template can encode, for example, transcription factors, growth factors, cytokines, cluster of differentiation (CD) molecules, interferons, interleukins, cell signaling proteins, protein receptors, protein hormones, antibody molecules, or long non-coding RNAs involved in cellular differentiation or maintenance thereof.

In some embodiments, the exogenous gene of interest or exogenous sequence of interest comprises a nucleic acid sequence encoding a chimeric antigen receptor (CAR). For example, the CAR encoded by the exogenous sequence of interest can comprise an extracellular ligand-binding domain having specificity for a tumor antigen. The tumor antigens disclosed herein can be any antigen that is preferentially expressed on tumor cells which allows for specific targeting of cancer cells.

As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, insulin growth factor (IGF1)-1, IGF-II, IGFI receptor, mesothelin, a maj or histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the Al domain of tenascin-C(TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a maj or histocompatibility complex (MHC) molecule, BCMA (CD269, TNFRSF 17), CS1, or a virus-specific surface antigen such as an HIV-specific antigen (such as HIV gp120); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers. In a particular embodiment of the present disclosure, the ligand-binding domain is specific for CD19.

In some embodiments, the extracellular domain of a chimeric antigen receptor further comprises an autoantigen (see, Payne et al. (2016) Science, Vol. 353 (6295): 179-184), which can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs).

In some embodiments, the extracellular domain of a chimeric antigen receptor can comprise a naturally-occurring ligand for an antigen of interest, or a fragment of a naturally-occurring ligand which retains the ability to bind the antigen of interest.

The CAR can be specific for any type of cancer cell. Such cancers can include, without limitation, carcinoma, lymphoma, sarcoma, blastomas, leukemia, cancers of B cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin's lymphoma. In certain embodiments, cancers of B cell origin include, without limitation, B lineage acute lymphoblastic leukemia, B cell chronic lymphocytic leukemia, B cell non-Hodgkin's lymphoma, and multiple myeloma.

A CAR can further include additional structural elements, including a transmembrane domain which is attached to the extracellular ligand-binding domain via a hinge or spacer sequence, and one or more intracellular signaling and/or co-stimulatory domains.

In some embodiments, a CAR comprises a transmembrane domain which links the extracellular ligand-binding domain or autoantigen with the intracellular signaling and co-stimulatory domains via a hinge or spacer sequence. The transmembrane domain can be derived from any membrane-bound or transmembrane protein. For example, the transmembrane polypeptide can be a subunit of the T-cell receptor (i.e., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. Alternatively the transmembrane domain can be synthetic and can comprise predominantly hydrophobic residues such as leucine and valine. In particular examples, the transmembrane domain is a CD8α transmembrane polypeptide.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence, or may be an entirely synthetic hinge sequence. In particular examples, a hinge domain can comprise a part of a human CD8 alpha chain, FcγRllla receptor or IgG1.

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. An intracellular signaling domain, such as CD3ζ, can provide an activation signal to the cell in response to binding of the extracellular domain. As discussed, the activation signal can induce an effector function of the cell such as, for example, cytolytic activity or cytokine secretion.

The intracellular domain of the CAR can include one or more intracellular co-stimulatory domains which transmit a co-stimulatory signal to promote cell proliferation, cell survival, and/or cytokine secretion after binding of the extracellular domain. Such intracellular co-stimulatory domains include those known in the art such as, without limitation, CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83, Ni, or N6.

In particular embodiments, the exogenous sequence of interest can encode an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest.

In other embodiments, the exogenous sequence of interest can encode the wild-type or modified version of an endogenous gene of interest.

In some embodiments, the methods disclosed herein encompass introducing an IRF3 inhibitor prior to, simultaneously with, or following a DNA template into a primary eukaryotic cell, wherein the DNA template comprises a nucleic acid molecule encoding a CAR or an exogenous T cell receptor.

The methods disclosed herein can be used for delivery of a DNA template comprising a nucleic acid sequence encoding a therapeutic protein to a primary eukaryotic cell to treat or prevent a disease. For example, the DNA template can be used to express a therapeutic gene in order to treat a gene deficiency disorder. Particularly appropriate genes for expression include those genes that are thought to be expressed at a less than normal level in the target cells of a subject mammal or animal. Particularly useful gene products include carbamoyl synthetase I, ornithine transcarbamylase, arginosuccinate synthetase, arginosuccinate lyase, and arginase. Other desirable gene products include fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase (also referred to as P-protein), H-protein, T-protein, Menkes disease copper-transporting ATPase, Wilson's disease copper-transporting ATPase, and CFTR (e.g., for treating cystic fibrosis).

The exogenous sequence of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, 85%, 90%, 95%, 99% or more sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

Exogenous sequences of interest can also encode a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like. In specific embodiments, the phenotypic marker allows for the selection of T cells having a specific chromosomal modification, such as introduction of a gene of interest or inactivation of an endogenous gene of interest.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

The DNA template can be introduced by any means described herein for introduction of a nucleic acid molecule into a primary cell. The DNA template can be introduced as a linear nucleic acid molecule or as a circular plasmid. In some embodiments, the DNA template is introduced as an expression cassette. Thus, in certain embodiments, expression cassettes or expression constructs are provided for the expression of at least one exogenous gene of interest, in a primary eukaryotic cell (e.g., a primary human T cell). Expression cassettes can be linear or can be located on a circular plasmid. The cassette can include 5′ and 3′ regulatory sequences operably linked to a polynucleotide provided herein encoding a gene of interest. “Operably linked” is intended to mean a functional linkage between two or more elements. For example, an operable linkage between a gene of interest as disclosed herein and a regulatory sequence (e.g., a promoter) is a functional link that allows for expression of the gene of interest. Operably linked elements may be contiguous or non-contiguous. When used to refer to the joining of two protein coding regions, by operably linked is intended that the coding regions are in the same reading frame.

The expression cassette can include in the 5′-3′ direction of transcription, a transcriptional and translational initiation region (i.e., a promoter), a nucleic acid sequence encoding a co-stimulatory domain, or active variant thereof, as disclosed herein, and a transcriptional and translational termination region (i.e., termination region) functional in cells, such as T cells. The regulatory regions (i.e., promoters, transcriptional regulatory regions, and translational termination regions) and/or a polynucleotide provided herein may be native/analogous to the host cell or to each other. Alternatively, the regulatory regions and/or a polynucleotide provided herein may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, the regulatory regions and/or a recombinant polynucleotide provided herein may be entirely synthetic.

The termination region may be native with the transcriptional initiation region, may be native with the operably linked polynucleotide, may be native with the cell host, or may be derived from another source (i.e., foreign or heterologous) to the promoter, the polynucleotide, the cell host, or any combination thereof. In preparing the expression cassette, the various DNA fragments may be manipulated, so as to provide for the DNA sequences in the proper orientation. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the expression cassettes provided herein. One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EGF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The promoters can be selected based on the desired outcome. It is recognized that different applications can be enhanced by the use of different promoters in the expression cassettes to modulate the timing, location and/or level of expression of the polynucleotides disclosed herein. Such expression constructs may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal. Synthetic promoters can also be utilized in the methods of the invention such as, for example, the JeT promoter.

### 2.5 Introduction of an Endonuclease

The methods disclosed herein for reducing IRF3 signaling can increase the total number of surviving primary cells, increase the number of gene-edited cells, increase gene editing efficiency, increase the number of cells comprising targeted insertion of a sequence of interest, and/or increase insertion frequency following introduction of a DNA template into a primary cell along with an endonuclease that is specific for a recognition sequence in the genome of the primary eukaryotic cell. In specific embodiments, the recognition sequence is located within an endogenous gene of interest. In specific embodiments, the DNA template and/or exogenous sequence of interest lacks substantial homology to the recognition sequence such that the endonuclease does not cleave the DNA template or any sequence contained therein.

Cleavage at recognition sequences can allow for NHEJ at the cleavage site and disrupted expression of the endogenous gene of interest. As used herein “gene editing” can refer to the cleavage of an endogenous recognition sequence in a primary eukaryotic cell and subsequent alteration of the cell genome during repair of the cleaved recognition sequence. An edit can result in a mutation comprising an insertion, deletion, or substitution of one or more nucleotides of the target sequence (i.e., an “indel”). Accordingly, “gene editing efficiency” can refer to the percentage of primary eukaryotic cells comprising an edit at the recognition sequence in the target site of the genome when compared to the percentage of primary eukaryotic cells following introduction of an IRF3 inhibitory molecule and a nuclease that cleaves the recognition sequence. For example, gene editing efficiency can be increased by about 1%, 2%, 3%, 4%, 5%, 6%, 8%, 10%, 12%, 15%, 20%, 25%, 30%, 35%, 40%, 50%, 60%, 70%, 80%, 90%, 100%, or more, by reducing IRF3 signaling.

Gene editing efficiency can be measured at any time after the reduction of IRF3 signaling or introduction of an IRF3 inhibitory molecule. In some embodiments, gene editing efficiency is measured after introduction of an IRF3 inhibitory molecule and introduction of a DNA template. In certain embodiments, gene editing efficiency is measured after introduction of an IRF3 inhibitory molecule, introduction of a DNA template as described herein, and introduction of a nucleic acid molecule encoding an engineered nuclease. For example, in some embodiments, gene editing efficiency is measured 6 hours, 12 hours, 18 hours, 2 days, 3 days, 4 days, 5 days, 6 days, 7 days, 8 days, 9 days, 10 days, 11 days, 12 days, 13 days, 14 days, 18 days, 21 days, or more following introduction of an IRF3 inhibitor. Although other molecules, such as a DNA template or nucleic acid molecule encoding an engineered nuclease may be introduced after the IRF3 inhibitor, the time periods recited herein for measurement of gene editing efficiency are calculated from the introduction of an IRF3 inhibitor.

In some embodiments, the total number of edited cells is increased following introduction of an endonuclease and introduction of an IRF3 signaling inhibitor when compared to the total number of edited cells resulting by introducing an endonuclease without reducing IRF3 signaling. In specific embodiments, the total number of edited cells following introduction of an endonuclease and introduction of an IRF3 signaling inhibitor can increase by 50, 100, 150, 200, 250, 500, 1000, 2000, 3000, 4000, 5000, 7500, 1×10⁴, 1×10⁵, 1×10⁶, 1×10⁷, 1×10⁸, 1×10⁹, 1×10¹⁰ cells, or more, compared to the number of cells resulting by introducing an endonuclease without reducing IRF3 signaling. In other specific embodiments, the total number of edited cells following introduction of an endonuclease and introduction of an IRF3 signaling inhibitor can increase by about 2 fold, about 3 fold, about 4 fold, about 5 fold, about 10 fold, about 20 fold, about 50 fold, about 100 fold, or more. The total number of cells can be measured by any means in the art.

The total number of edited cells can be measured at any time after the reduction of IRF3 signaling or introduction of an IRF3 inhibitory molecule. In some embodiments, the total number of edited cells is measured after introduction of an IRF3 inhibitory molecule and introduction of a DNA template. In certain embodiments, the total number of edited cells is measured after introduction of an IRF3 inhibitor molecule, introduction of a DNA template as described herein, and introduction of a nucleic acid molecule encoding an engineered nuclease. For example, in some embodiments, the total number of edited cells is measured 6 hours, 12 hours, 18 hours, 2 days, 3 days, 4 days, 5 days, 6 days, 7 days, 8 days, 9 days, 10 days, 11 days, 12 days, 13 days, 14 days, 18 days, 21 days, or more, following introduction of an IRF3 inhibitor. Although other molecules, such as a DNA template or nucleic acid molecule encoding an engineered nuclease may be introduced after the IRF3 inhibitor, the time periods recited herein for measurement of the total number of edited cells are calculated from the introduction of an IRF3 inhibitor.

Additionally, cleavage at such recognition sequences can further allow for homologous recombination of an exogenous sequence of interest (e.g., exogenous gene of interest) directly into a target site comprising the recognition sequence. For example, in some embodiments, cleavage of a recognition sequence by an endonuclease (e.g., an engineered meganuclease), can allow for insertion of an exogenous sequence of interest flanked by 5′ homology arm and 3′ homology arm, on a DNA template, to promote recombination of the exogenous sequence of interest into the cell genome at the nuclease cleavage site.

Thus, expression cassettes and DNA templates disclosed herein can have a 5′ homology arm comprised of a nucleic acid sequence 5′ upstream of the coding sequence of the exogenous sequence of interest that corresponds to an endogenous nucleotide sequence. Likewise, expression cassettes disclosed herein can have a 3′ homology arm comprised of a nucleic acid sequence 3′ downstream of the coding sequence of the exogenous sequence of interest that corresponds to an endogenous nucleotide sequence. The 5′ and 3′ homology arms thereby promote homologous recombination of the exogenous sequence of interest into the chromosome of the eukaryotic cell at the cleaved endogenous recognition site.

In specific embodiments, the 5′ and 3′ homology arms have at least 70%, at least 80%, at least 85%, at least 90%, at least 91%, at least 92%, at least 93%, at least 94%, at least 95%, at least 96%, at least 97%, at least 98%, at least 99%, or 100% sequence identity to the corresponding sequence in the chromosome of the eukaryotic cell. The 5′ and 3′ homology arms can comprise at least 10, at least 20, at least 30, at least 40, at least 50, at least 75, at least 100, at least 150, at least 200, at least 250, at least 500 base pairs, or any length sufficient to promote recombination of the exogenous sequence of interest into the eukaryotic chromosome.

Thus, insertion frequency of any exogenous sequence of interest or exogenous gene of interest into a target site in the genome of a primary eukaryotic cell can be increased by reducing IRF3 signaling. As used herein, “insertion frequency” can refer to the percentage of primary eukaryotic cells in a population comprising a sequence of interest inserted into a target site of the genome. For example, insertion frequency can increase by about 1%, 2%, 3%, 4%, 5%, 6%, 8%, 10%, 12%, 15%, 20%, 25%, 30%, 35%, 40%, 50%, 60%, 70%, 80%, 90%, 100%, or more, when compared to appropriate control cells. Insertion frequency can also increase by about 2 fold, about 3 fold, about 4 fold, about 5 fold, about 10 fold, about 20 fold, about 50 fold, about 100 fold, or more, when compared to appropriate control cells.

In some embodiments, the total number of cells comprising an exogenous sequence of interest inserted into a target site is increased following introduction of an endonuclease and exogenous sequence of interest along with introduction of an IRF3 signaling inhibitor when compared appropriate control cells. In specific embodiments, the total number of cells comprising targeted insertion of the sequence of interest can increase by 50, 100, 150, 200, 250, 500, 1000, 2000, 3000, 4000, 5000, 7500, 1×10⁴, 1×10⁵, 1×10⁶, 1×10⁷, 1×10⁸, 1×10⁹, 1×10¹⁰ cells, or more, compared to appropriate control cells. In other embodiments, the total number of cells comprising targeted insertion of a sequence of interest can increase by about 2 fold, about 3 fold, about 4 fold, about 5 fold, about 10 fold, about 20 fold, about 50 fold, about 100 fold, or more, when compared to appropriate control cells.

The total number of cells comprising targeted insertion of a sequence of interest can be measured at any time after the reduction of IRF3 signaling or introduction of an IRF3 inhibitory molecule. In some embodiments, the total number of cells is measured after introduction of an IRF3 inhibitory molecule and introduction of a DNA template. In certain embodiments, the total number of cells is measured after introduction of an IRF3 inhibitory molecule, introduction of a DNA template as described herein, and introduction of a nucleic acid molecule encoding an engineered nuclease. For example, in some embodiments, the total number of cells expressing an exogenous sequence of interest is measured 6 hours, 12 hours, 18 hours, 2 days, 3 days, 4 days, 5 days, 6 days, 7 days, 8 days, 9 days, 10 days, 11 days, 12 days, 13 days, 14 days, 18 days, 21 days, or more, following introduction of an IRF3 inhibitor. Although other molecules, such as a DNA template or nucleic acid molecule encoding an engineered nuclease may be introduced after the IRF3 inhibitor, the time periods recited herein for measurement of the total number of cells expressing an exogenous sequence of interest are calculated from the introduction of an IRF3 inhibitor.

In some embodiments, the methods disclosed herein can take advantage of the site specificity of certain endonucleases to cleave at least one recognition sequence in an endogenous polynucleotide of interest (e.g., endogenous gene of interest). Following cleavage, the site can be edited or have an exogenous gene of interest inserted in the target site. Any endonuclease that specifically or preferentially cleaves the corresponding recognition sequences can be used in the methods and compositions disclosed herein. By using endonucleases that specifically and preferentially cleave the endogenous recognition sequences, cleavage at sites other than the recognition sequences is minimized and efficiency of cleavage is thereby increased. Accordingly, the endonucleases for cleavage of the recognition sequences and endogenous recognition sequences disclosed herein can be a meganuclease, a zinc finger nuclease, a TALEN, a compact TALEN, a megaTAL, or a CRISPR/Cas. In some embodiments, the methods disclosed herein encompass introducing an IRF3 inhibitor prior to, simultaneously with, or after, a DNA template into a primary eukaryotic cell, and further introducing into the primary eukaryotic cell an endonuclease or a nucleic acid molecule encoding an endonuclease.

Methods for producing engineered, site-specific endonucleases are known in the art. For example, zinc-finger nucleases (ZFNs) can be engineered to recognize and cut pre-determined sites in a genome. ZFNs are chimeric proteins comprising a zinc finger DNA-binding domain fused to the nuclease domain of the FokI restriction enzyme. The zinc finger domain can be redesigned through rational or experimental means to produce a protein which binds to a pre-determined DNA sequence ˜18 basepairs in length. By fusing this engineered protein domain to the FokI nuclease, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)). Likewise, TAL-effector nucleases (TALENs) can be generated to cleave specific sites in genomic DNA. Like a ZFN, a TALEN comprises an engineered, site-specific DNA-binding domain fused to the FokI nuclease domain (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease. Unlike FokI, I-TevI does not need to dimerize to produce a double-strand DNA break so a Compact TALEN is functional as a monomer. Thus, it is possible to co-express two Compact TALENs in the same cell in the methods disclosed herein.

Engineered endonucleases based on the CRISPR/Cas9 system are also know in the art (Ran, et al. (2013) Nat Protoc. 8:2281-2308; Mali et al. (2013) Nat Methods. 10:957-63). A CRISPR endonuclease comprises two components: (1) a caspase effector nuclease, typically microbial Cas9 or Cpf1; and (2) a short “guide RNA” comprising a ˜20 nucleotide targeting sequence that directs the nuclease to a location of interest in the genome. By expressing multiple guide RNAs in the same cell, each having a different targeting sequence, it is possible to target DNA breaks simultaneously to multiple sites in in the genome. Thus, CRISPR/Cas9 nucleases, CRISPR/Cpf1 nucleases, and the like, are suitable for the present invention. The primary drawback of CRISPR systems is the reported high frequency of off-target DNA breaks, which could limit the utility of the system for treating human patients (Fu, et al. (2013) Nat Biotechnol. 31:822-6).

In specific embodiments of the methods and compositions disclosed herein, the DNA break-inducing agent is an engineered homing endonuclease (also called a “meganuclease”). Homing endonucleases are a group of naturally-occurring nucleases which recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Homing endonucleases are commonly grouped into four families: the LAGLIDADG (SEQ ID NO: 1) family, the GIY-YIG family, the His-Cys box family and the HNH family. These families are characterized by structural motifs, which affect catalytic activity and recognition sequence. For instance, members of the LAGLIDADG (SEQ ID NO: 1) family are characterized by having either one or two copies of the conserved LAGLIDADG (SEQ ID NO: 1) motif (see Chevalier et al. (2001), Nucleic Acids Res. 29(18): 3757-3774). The LAGLIDADG (SEQ ID NO: 1) homing endonucleases with a single copy of the LAGLIDADG (SEQ ID NO: 1) motif form homodimers, whereas members with two copies of the LAGLIDADG (SEQ ID NO: 1) motif are found as monomers.

I-CreI is a member of the LAGLIDADG (SEQ ID NO: 1) family of homing endonucleases which recognizes and cuts a 22 basepair recognition sequence in the chloroplast chromosome of the algae Chlamydomonas reinhardtii. Genetic selection techniques have been used to modify the wild-type I-CreI cleavage site preference (Sussman et al. (2004), J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58). More recently, a method of rationally-designing mono-LAGLIDADG (SEQ ID NO: 1) homing endonucleases was described which is capable of comprehensively redesigning I-CreI and other homing endonucleases to target widely-divergent DNA sites, including sites in mammalian, yeast, plant, bacterial, and viral genomes (WO 2007/047859).

As first described in WO 2009/059195, I-CreI and its engineered derivatives are normally dimeric but can be fused into a single polypeptide using a short peptide linker that joins the C-terminus of a first subunit to the N-terminus of a second subunit (Li, et al. (2009) Nucleic Acids Res. 37:1650-62; Grizot, et al. (2009) Nucleic Acids Res. 37:5405-19.) Thus, a functional “single-chain” meganuclease can be expressed from a single transcript. By delivering genes encoding two different single-chain meganucleases to the same cell, it is possible to simultaneously cut two different sites.

Engineered nucleases of the invention can be delivered into a cell in the form of protein or, preferably, as a nucleic acid encoding the engineered nuclease. Such nucleic acid can be DNA (e.g., circular or linearized plasmid DNA or PCR products) or RNA. For embodiments in which the engineered nuclease coding sequence is delivered in DNA form, the nucleic acid sequence encoding the engineered nuclease should be operably linked to a promoter to facilitate transcription of the meganuclease gene. Mammalian promoters suitable for the invention include constitutive promoters such as the cytomegalovirus early (CMV) promoter (Thomsen et al. (1984), Proc Natl Acad Sci USA. 81(3):659-63) or the SV40 early promoter (Benoist and Chambon (1981), Nature. 290(5804):304-10) as well as inducible promoters such as the tetracycline-inducible promoter (Dingermann et al. (1992), Mol Cell Biol. 12(9):4038-45). In some embodiments, DNA encoding an engineered nuclease can be delivered on a DNA template or expression cassette as described herein.

In another particular embodiment, genes encoding an endonuclease of the invention can be introduced into a cell using a linearized DNA template. In some examples, a plasmid DNA encoding an endonuclease can be digested by one or more restriction enzymes such that the circular plasmid DNA is linearized prior to being introduced into a cell.

In some embodiments, mRNA encoding the engineered nuclease is delivered to the cell because this reduces the likelihood that the gene encoding the engineered nuclease will integrate into the genome of the cell. Such mRNA encoding an engineered nuclease can be produced using methods known in the art such as in vitro transcription. In some embodiments, the mRNA is capped using 7-methyl-guanosine. In some embodiments, the mRNA may be polyadenylated.

In particular embodiments, an mRNA encoding an engineered nuclease of the invention can be a polycistronic mRNA encoding two or more nucleases which are simultaneously expressed in a cell. In some embodiments, a polycistronic mRNA can encode two or more nucleases which target different recognition sequences in the primary cell genome, such that the genome of the primary cell is cleaved at multiple sites. In some embodiments, a polycistronic mRNA can encode two or more nucleases and at least one additional protein which induces a therapeutically beneficial effect in the cell. A polycistronic mRNA of the invention can comprise any element known in the art to allow for the translation of two or more genes from the same mRNA molecule including, but not limited to, an IRES element, a T2A element, a P2A element, an E2A element, and an F2A element. In particular embodiments, the polycistronic mRNA is a bicistronic mRNA encoding two engineered nucleases, a tricistronic mRNA encoding three engineered nucleases, or a quadcistronic mRNA encoding four engineered nucleases, wherein the nucleases encoded by each mRNA have specificity for different recognition sequences in the primary cell genome.

Purified nuclease proteins can be delivered into cells to cleave genomic DNA, which allows for homologous recombination or non-homologous end-joining at the cleavage site with a sequence of interest, by a variety of different mechanisms known in the art.

In some embodiments, engineered nuclease proteins, or DNA/mRNA encoding engineered nucleases, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, engineered nucleases, or DNA/mRNA encoding engineered nucleases, are coupled covalently or non-covalently to an antibody that recognizes a specific cell-surface receptor expressed on target cells such that the nuclease protein/DNA/mRNA binds to and is internalized by the target cells. Alternatively, engineered nuclease protein/DNA/mRNA can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11): 1491-508).

In some embodiments, engineered nuclease proteins, or DNA/mRNA encoding engineered nucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the recombinant meganuclease proteins, mRNA, or DNA can be attached to or encapsulated with the nanoparticle core. This increases the copy number of the protein/mRNA/DNA that is delivered to each cell and, so, increases the intracellular expression of each engineered nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the engineered nucleases or DNA/mRNA encoding the engineered nucleases, are encapsulated within liposomes or complexed using cationic lipids (see, e.g., Lipofectamine, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, and facilitate cellular uptake and delivery efficiency through fusion with and/or disruption of the cellular membranes of the cells.

In some embodiments, engineered nuclease proteins, or DNA/mRNA encoding engineered nucleases, are encapsulated within polymeric scaffolds (e.g., PLGA) or complexed using cationic polymers (e.g., PEI, PLL) (Tamboli et al. (2011) Ther Deliv. 2(4): 523-536).

In some embodiments, engineered nuclease proteins, or DNA/mRNA encoding engineered nucleases, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions outside of the cell.

In some embodiments, engineered nuclease proteins, or DNA/mRNA encoding engineered nucleases, are formulated into an emulsion or a nanoemulsion (i.e., having an average particle diameter of <1 nm) for delivery to the cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in US Patent Application Nos. 2002/0045667 and 2004/0043041, and U.S. Pat. Nos. 6,015,832, 6,506,803, 6,635,676, and 6,559,189, each of which is incorporated herein by reference in its entirety.

In some embodiments, engineered nuclease proteins, or DNA/mRNA encoding engineered nucleases, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability and reduce nonspecific interactions.

According to the methods disclosed herein, DNA template and/or any nucleic acid molecule encoding an engineered nuclease can be introduced into a primary eukaryotic cell before, after, or simultaneously with, introduction of an IRF3 inhibitor.

The methods and compositions described herein allow for more efficient editing of endogenous genomic sequences, and/or insertion of exogenous polynucleotide sequences into an endogenous recognition site, when IRF3 signaling is reduced and an endonuclease is expressed in the primary cell. Cleavage at such recognition sequences can allow for NHEJ at the cleavage site and disrupted expression of a target gene in the chromosome of the primary eukaryotic cell, or can provide an opportunity for homologous recombination of an exogenous sequence of interest into the cleaved recognition sequence.

In specific embodiments, an endogenous gene of interest encodes an endogenous polypeptide, such as a T cell receptor, or component thereof. For example, endogenous recognition sequences can be located within the human T cell receptor alpha constant region gene, leading to reduced expression and/or function of the T cell receptor at the cell surface when the endogenous recognition sequence is cleaved. Additionally, cleavage at such recognition sequences can further allow for homologous recombination of an exogenous sequence of interest directly into the TCR alpha constant region gene. In specific embodiments, an exogenous sequence of interest encoding a chimeric antigen receptor (CAR) is inserted into a cleaved recognition sequence in a TCR alpha constant region gene following reduction of IRF3 signaling.

In particular embodiments, the exogenous sequence of interest can encode a CAR or an exogenous T cell receptor. In other embodiments, the exogenous sequence of interest can encode a wild-type or modified version of an endogenous gene of interest.

### 2.6 Characterization of Phenotype

The methods disclosed herein can be utilized for high throughput screening of primary human T cells that express a chimeric antigen receptor (CAR) or exogenous T cell receptor (TCR). In this sense, high throughput means that the screening of such genetically-modified T cells can be done rapidly and in greater numbers when compared to previous methods described in the art, which could require the generation of AAVs in order to evaluate each CAR or exogenous TCR construct of interest. The present methods enable one of skill to rapidly characterize a number of phenotypes of such T cells, allowing for selection of promising constructs for further pre-clinical and clinical evaluation.

In certain embodiments, the methods can be used to determine the frequency of cell surface expression of a CAR or an exogenous TCR on a genetically-modified T cell. Methods for such determination can include, for example, immunostaining as described herein, or any other methods known in the art for detecting a cell-surface CAR or exogenous TCR.

In certain embodiments, the methods can be used to determine the memory phenotype of primary human T cells expressing a CAR or an exogenous TCR. T cells can have, for example, a central memory phenotype, a transitional memory phenotype, or an effector memory phenotype. Central memory phenotype T cells are characterized by expression of CD62L, CCR7, and low levels of CD45RO expression. Transitional memory phenotype T cells are characterized by expression of CD62L, CCR7, and high levels of CD45RO expression. Effector memory phenotype T cells are characterized by expression of CD45RO and a lack of CD62L and CCR7 expression.

In certain embodiments, the methods can be used to determine the CD4⁺ to CD8⁺ ratio of the primary human T cells expressing a CAR or an exogenous TCR. Methods for determining populations of CD4⁺ and CD8⁺ cells include those described in the examples herein and other methods known in the art.

In other embodiments, the methods can be used to quantify exhaustion markers expressed by the primary human T cells expressing a CAR or an exogenous TCR. Exhaustion markers are upregulated on activated T cells and sustained expression of such proteins allow for the dampening of the immune response during repetitive antigen exposure and subsequent engagement of the T-cell receptor. Cell-surface markers of T cell exhaustion can include, but are not limited to, TIM-3, PD-1, and/or LAG-3. The presence of exhaustion markers can be determined by methods including, but not limited to, flow cytometric analysis.

In some embodiments, the methods can be used to quantify antigen-independent and/or antigen-induced secretion of cytokines by primary human T cells expressing a CAR or an exogenous TCR. Such genetically-modified T cells can secrete cytokines in the absence of antigen (i.e., antigen-independent signaling) or upon CAR or TCR activation via antigen binding. In some examples, secreted cytokines that can be detected include, but are not limited to, interferon-gamma, interleukin-2, tumor necrosis factor alpha, granzyme B, interleukin-6, and perforin.

In some embodiments, the methods can be used to determine antigen-independent phosphorylated protein expression by primary human T cells expressing a CAR or an exogenous TCR. Engagement of a cell surface receptor with its cognate antigen results in intracellular transduction of the activating signal. One common mechanism by which that signal is transduced is through the phosphorylation/de-phosphorylation of proteins involved in the signaling cascade. The phosphorylation of proteins by kinases results in inherent changes in the structure of those proteins, often allowing for improved binding to interacting partners for propagation of the activating signal. Phosphorylation of proteins at specific residues are indicative of an activated or inhibited form of that protein. Importantly, activation of a signaling cascade in the absence of antigen-exposure can lead to chronic, low level signaling that often results in that cell becoming more quickly exhausted once exposed to its antigen. Therefore, analysis of phosphorylated proteins can be used to gauge the activation status of the cell, whether after, or in the absence, of antigen. Phosphorylated proteins that can be detected by the methods can include, but are not limited to, CD3ζ, extracellular signal-regulated kinase (ERK), RAC-alpha serine/threonine-protein kinase (AKT1), and p38.

In some embodiments, the methods can be used to evaluate cytotoxicity of primary human T cells expressing a CAR or an exogenous TCR against antigen-bearing target cells. Methods for determining cytotoxicity include, for example, methods similar to those described in U.S. Pat. No. 9,889,160, and other methods known in the art.

In some embodiments, the methods can be used to determine antigen-independent and/or antigen-induced proliferative capacity of primary human T cells expressing a CAR or an exogenous TCR. One consequence of receptor engagement with antigen and subsequent transduction of an activation signal as described above is proliferation of the responding cell. The ability of a specific cell to proliferate and make clones of itself is paramount to the clearance of an invading pathogen during an immune response. Furthermore, analysis of proliferative capacity, or the number of times a cell can divide, has been shown to correlate with the activation status of the cell itself. Specifically, cells that are less activated upon initial exposure to antigen will proliferative more times than a cell that is more highly exhausted upon antigen engagement. Importantly, proliferation in the absence of antigen exposure leads to a dampening of immune potential of those cells when engaged. A common methodology for analyzing proliferative capacity is by labeling the surface of cells with a fluorescent protein known as a succinimidyl ester. Those labeled cells are then co-cultured with different target populations, expressing either the antigen to which those cells respond or a non-specific decoy antigen. Upon antigen engagement cells will proliferate, with each daughter cells obtaining half of the fluorescent dye on their surface. As cells continue to divide, the amount of fluorescent dye decreases accordingly. The number of times an individual cell divides and the proliferative capacity of the cell population as a whole can then be determined by flow cytometric analysis of the residual intensity of the fluorescent protein on the cell surface.

In some embodiments, the methods can be used to determine exhaustion of the primary human T cells expressing a CAR or an exogenous TCR after repeated antigen encounter. Activation, and corresponding proliferation of, T cells is inherent to the ability of those cells to eliminate pathogens. During this process, antigen-specific T cells do not interact with target populations only one time but are capable of engaging targets continually until the pathogen has been eradicated, or until that individual T cell becomes exhausted. Continual exposure to antigen, and subsequent transduction of activating signals within the cell, results in a shift in intracellular protein expression. This causes a change in cell surface markers that pushes them through the progression of memory subsets as described on page 52. Furthermore, alterations in intracellular proteins can lead to the upregulation of markers on the surface that are indicative of exhaustion. Engagement of these receptors with their corresponding ligands leads to substantial shifts in the potency of the cell, including reductions in effector cytokine secretion, diminished proliferation, and altered cellular metabolism. Collectively, this causes a cell to become functionally inert. Methods for determining antigen-induced exhaustion include, for example, an in vitro assay known as a “stress-test”. During this assay, T cells expressing a CAR or an exogenous TCR are co-cultured with target cells at a fixed ratio. Without further intervention, the CAR T cells would eliminate the target population over time. In the context of a stress-test, however, fresh target cells are seeded in the wells containing the antigen-specific T cells at specific time points. This ensures that a relevant target population is always present and that the T cells are continually engaged. This process, therefore, pushes the responding T cells through different memory T cell subsets and results in the upregulation of exhaustion markers that are analyzed by flow cytometry. In addition, correlative reductions in cytokine expression and proliferative capacity can also be measured. Overall, this co-culture method allows for the temporal analysis of antigen-specific T cell exhaustion.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Inhibition of the IRF3 Pathway to Reduce Linear and Plasmid DNA-Induced Cytotoxicity in Primary Human T Cells

### Purpose

The purpose of this experiment was to determine the effect of inhibiting the IRF3 signaling pathway on the cytotoxic effect of either linear or plasmid DNA transfection into primary cells, particularly primary human T cells. Specifically, these experiments utilize siRNA directed to IRF3 or STING.

### Materials and Methods

In these experiments, IRF3 or STING were knocked down in primary human T cells using siRNA. Smartpools and individual siRNAs specific for human IRF3 and STING were purchased commercially from Dharmacon (Lafayette, Colo.). siRNAs were re-suspended according to the manufacturers recommendations and frozen at −20° C. for downstream applications. The sequences for IRF3 and STING siRNAs can be found in Table 1 and Table 2 below:

TABLE 1
IRF3 siRNA smartpool

| siRNA | Sequence | SEQ ID NO: |
|---|---|---|
| IRF3-1 | CGAGGCCACUGGUGCAUAU | 2 |
| IRF3-2 | CCAGACACCUCUCCGGACA | 3 |
| IRF3-3 | GGAGUGAUGAGCUACGUGA | 4 |
| IRF3-4 | AGACAUUCUGGAUGAGUUA | 5 |

TABLE 2
STING siRNA smartpool:

| siRNA | Sequence | SEQ ID NO: |
|---|---|---|
| STING-1 | UCAUAAACUUUGGAUGCUA | 6 |
| STING-2 | CGAACUCUCUCAAUGGUAU | 7 |
| STING-3 | AGCUGGGACUGCUGUUAAA | 8 |
| STING-4 | GCAGAUGACAGCAGCUUCU | 9 |

Plasmid and linearized DNA constructs used in these experiments comprised a cassette which expresses an anti-CD19 scFv chimeric antigen receptor (CAR) with an incorporated human CD34 epitope tag, which allows for CAR detection.

In addition to the plasmid/linear DNA constructs and the siRNAs, primary human T cells were transfected with mRNA encoding a genetically-engineered meganuclease referred to as TRC 1-2x.87 EE (i.e., the TRC nuclease). The TRC nuclease recognizes and cleaves a recognition sequence present within the T cell receptor alpha constant region (TRAC) gene, where it produces a cleavage site. This cleavage site can be repaired by non-homologous end joining (NHEJ), which results typically results in an insertion or deletion (“indel”) at the cleavage site and a knockout of the T cell receptor. Alternatively, when an appropriate DNA template is present, the template can be inserted into the cleavage site by homologous recombination. In the present experiments, the CAR coding region in the plasmid/linear DNA constructs was flanked at both ends by homology arms, which are homologous to the regions upstream and downstream of the TRC nuclease cleavage site, thus allowing for targeted insertion of the CAR coding sequence into the TRAC gene by homologous recombination (i.e., homology directed repair, or HDR). This targeted insertion allows for both expression of the CAR and knockdown of T cell receptor expression.

Primary human T cells were magnetically isolated by CD3+ positive selection out of a mixed cell blood product obtained from a healthy donor using the EasySep release human CD3 positive selection kit (Stemcell Technologies, Vancouver, Canada). After isolation, cells were counted, aliquoted, and frozen in liquid nitrogen for future applications. Prior to nucleofection, frozen CD3+ T cells were thawed, washed twice in T cell media and rested for 4 hours in T cell media. Cells were then manually counted and stimulated for 72 hours with Immunocult (Stemcell Technologies, Vancouver, Canada) in T cell media supplemented with IL-2 at a concentration of 1×10⁶ cells/ml. Stimulated CD3⁺ T cells were centrifuged at 1300 RPM for 4 minutes to pellet and the supernatant was removed. T cells were subsequently washed, spun as above, and re-suspended in fresh T cell media. Automated cell counts and cell viabilities were acquired using the Countess II FL (Life Technologies, Carlsbad, Calif.). After counting, cells were spun once more, washed with room temperature PBS, and re-suspended in P3 buffer (Lonza, Basel Switzerland) at a concentration of 1×10⁶ viable cells/20 ul of P3 buffer.

DNA, mRNA, and/or siRNA was added to appropriate tubes prior to the addition of T cells in the following order. For siRNA containing conditions, 1.25 ul or 2.5 ul siRNA for specific STING or IRF3 variants or smartpools respectively (Dharmacon) was added from a stock solution frozen at 20° C. to tubes. Linearized or plasmid DNA was then added at a concentration of 1 ug DNA/condition, followed by the addition of 1 ug mRNA nuclease/condition from frozen stock solutions, and, lastly, 20 ul of T cells (1×10⁶ viable cells) in P3 buffer. Material was briefly mixed within the tube and transferred to individual wells of a 16-well nucleocuvette strip (Lonza). After addition of all samples, the nucleocuvette strip was placed within the 4-D nucleofector (Lonza) and cells were nucleofected using the “human T cells, stimulated” program.

Post-nucleofection, cells were rested at room temperature for 5 minutes. 80 ul of 37° C. pre-warmed T cell media supplemented with 30 ng/ml IL-2 was added to each cuvette well and cells were rested for an additional 10 minutes at room temperature. Samples in ˜100 ul total volume were subsequently transferred out of the nucleocuvette strip to individual wells of a 48-well plate. To each well was added 400 ul of additional T cell media with IL-2, for a total volume of 500 ul/sample. The plate was covered and cells were placed in a 37° C. incubator for 72 hours (i.e., 3 day time point).

After incubation, cell numbers and viabilities were assessed using the Countess II FL (Life Technologies). Remaining cells were pipetted off the tissue culture plate and added to individual 15 ml conical tubes. Cells were spun at 1300 RPM for 4 minutes to pellet in a tabletop centrifuge and the supernatant was removed. Remaining cells were re-suspended in fresh T cell media supplemented with IL-2 at a concentration of 1×10⁶ viable cells/ml and plated in a new sterile 48-well plate, which was placed in a 37° C. incubator for an additional 48 hours (i.e., 5 day time point).

At day 5 post-nucleofection, cell numbers and viabilities were assessed using the Countess II FL (Life Technologies) as previously done at day 3. Total cell numbers at each time point were graphed for comparison between sample conditions.

### Results

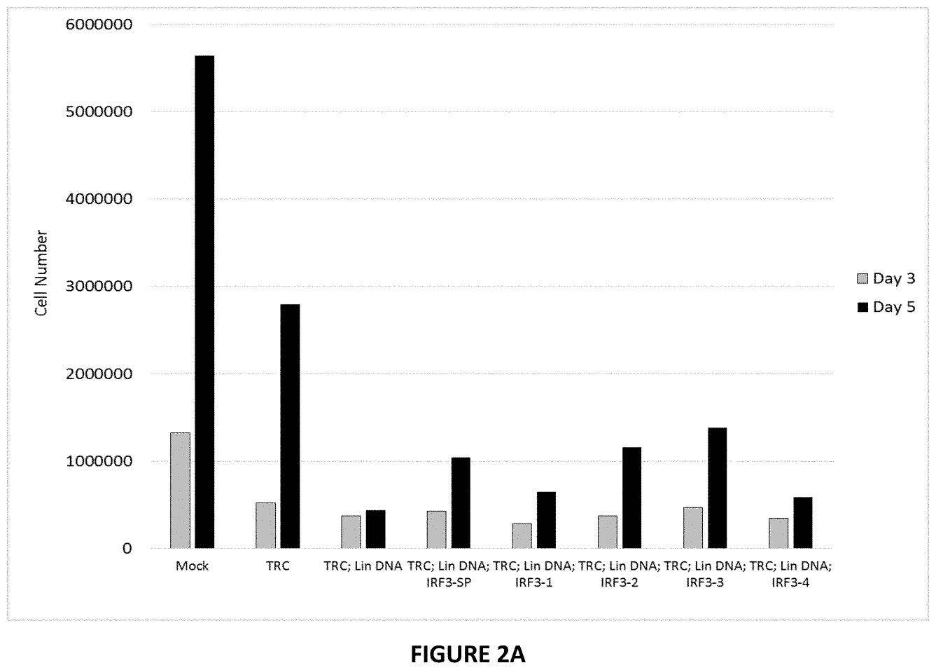

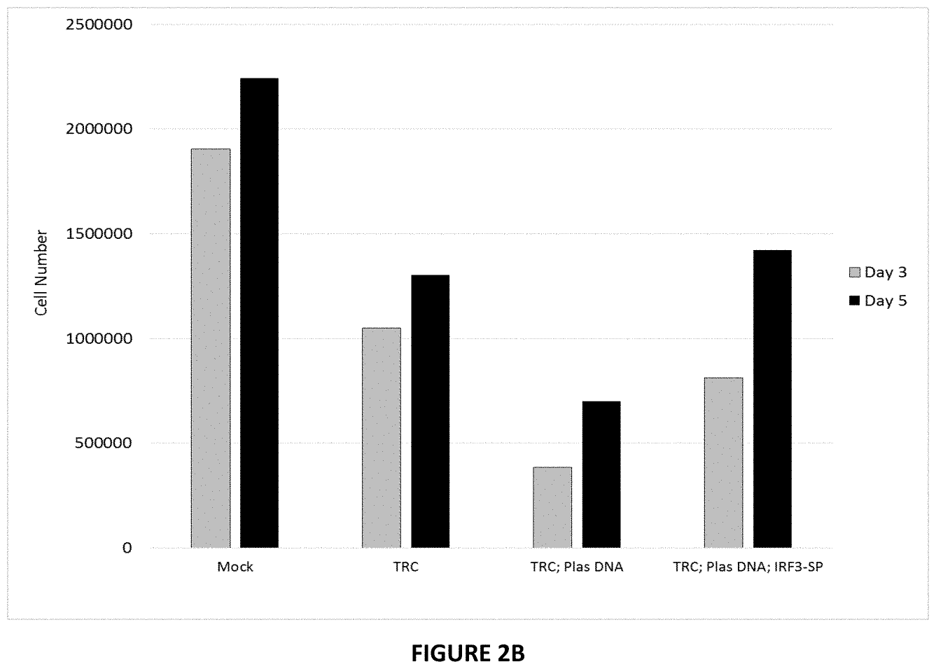

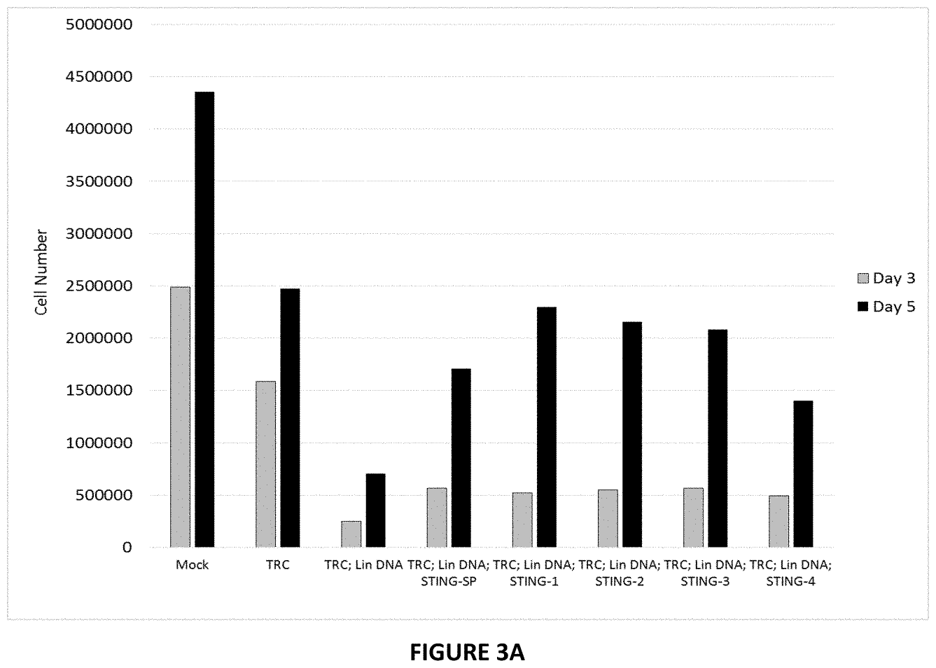

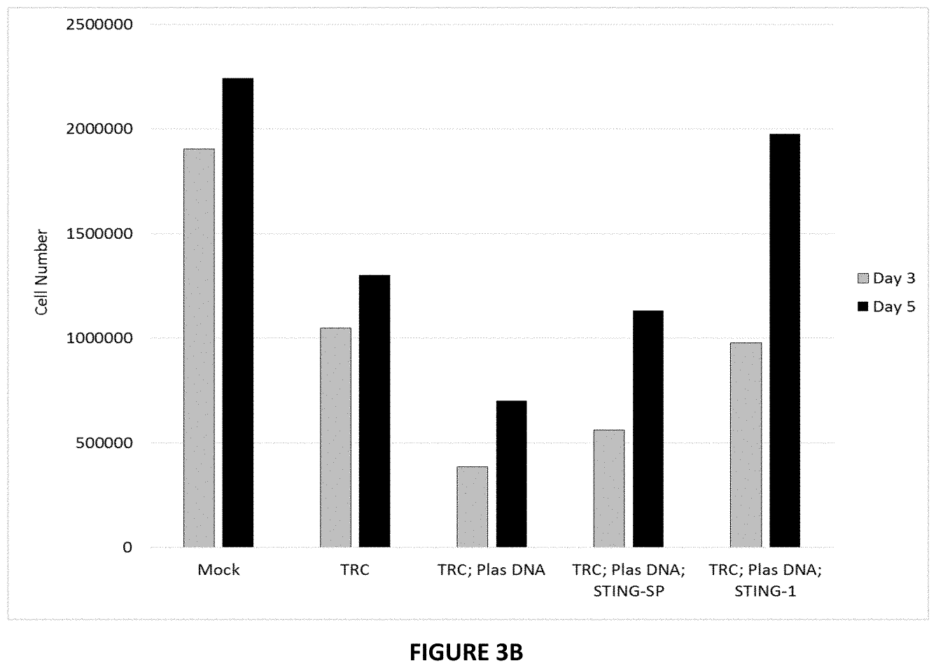

Compared to mock or mRNA-only nucleofected T cells, co-nucleofection with exogenous DNA, either plasmid or linear, and mRNA drastically reduced T cell numbers post-nucleofection. Specifically, in cells co-nucleofected with linearized DNA and mRNA, reduction in cell numbers ranged from 29-84% at day 3 and 71-84% at day 5 compared to mRNA nucleofected cells alone (FIGS. 2A and 3A), while co-nucleofection with plasmid DNA and mRNA resulted in a 63% reduction in cell number at day 3 and a 46% reduction at day 5, respectively (FIGS. 2B and 3B).

Addition of siRNA specific for IRF3 prior to nucleofection increased cell number at various time points post-nucleofection. Specifically, when IRF3 smartpool siRNA, which is a combination of four different siRNA variants, was utilized, cell numbers increased 16% at day 3 and 138% at day 5 post-nucleofection with linearized DNA (FIG. 2A). By comparison, cells co-nucleofected with plasmid, rather than linear, DNA resulted in over a 2-fold increase in cell number at day 3 and day 5 when IRF3 smartpool siRNA was added compared to no siRNA controls (FIG. 2B). Furthermore, use of individual siRNA variants resulted in similar increases in cell number post-nucleofection, confirming the results seen with the smartpool. Of note, co-nucleofection with IRF3-2 or IRF3-3 siRNA resulted in a 165% and 216% increase in cell number at day 5 compared to linear DNA and mRNA co-nucleofected control cells (FIG. 2A).

The use of STING siRNA showed a similar effect to IRF3 siRNA by reducing cellular cytotoxicity and increasing cell number post-nucleofection. STING siRNA added during nucleofection with linear DNA showed a consistent effect on cell number at day 3 post-nucleofection, ranging from 1.97-2.28-fold increase regardless of whether smartpool or individual variant siRNA was used. Strikingly, at day 5 post-nucleofection cell numbers were 1.99-3.27-fold higher compared to no siRNA controls, with highest cell numbers recovered from cell co-nucleofected with STING-1 siRNA (FIG. 3A). In the context of plasmid DNA, addition of STING smartpool siRNA resulted in a 46% and 62% increase in cell number at day 3 and 5 post-nucleofection respectively compared to conditions in the absence of siRNA. Moreover, the use of STING-1 variant siRNA, identified through our screen as the most beneficial siRNA of the smartpool constituents, enhanced cell numbers a further 74% compared to the use of the smartpool at our two identified time points (FIG. 3B).

### Conclusions

The addition of siRNA specific for IRF-3 or STING to the reaction mixture prior to nucleofection of T cells with mRNA and DNA mitigates the loss in T cells compared to T cells nucleofected with mRNA and DNA alone. This suggests that dampening of the STING/IRF3 pathway is beneficial in reducing the cytotoxicity associated with the introduction of foreign cytosolic-resident DNA to primary human T cells.

### Example 2

### Effect of IRF3 Knockdown on Gene Editing and Insertion of an Exogenous Nucleic Acid by Homologous Recombination

### Purpose

The purpose of these experiments was to determine the effect of IRF3 knockdown on gene editing and targeted insertion by homologous recombination in primary cells, particularly in primary human T cells.

Multiple experiments were performed to examine the effect of IRF3 knockdown by siRNA on four different parameters: (i) total number of gene-edited cells, as measured by the number of CD3⁻ cells (indicator of T cell receptor knockout); (ii) percentage of cells that are gene-edited (i.e., percent CD3⁻ cells within the population); (iii) total number of CD3⁻/CAR⁺ cells (indicator that the DNA template was inserted into the TRC nuclease cleavage site by homologous recombination); and (iv) percentage of cells that are CD3⁻/CAR⁺ (i.e., percent CD3⁻/CAR⁺ cells in the population).

The IRF3 siRNAs evaluated in each experiment are outlined in Table 3 below. In each experiment, cells were tested with groups including (i) mock nucleofection, (ii) TRC nuclease only, (iii) TRC nuclease with plasmid or linear DNA, and (iv) TRC nuclease, plasmid or linear DNA, and siRNA.

TABLE 3

| | DNA | | | |
|---|---|---|---|---|
| Experiment | Template | Target | siRNAs | Figure |
| 1 | Linear | IRF3 | IRF3 Smartpool, IRF-1, IRF-2, | 4 |
| | | | IRF-3, IRF-4 | |
| 2 | Linear | IRF3 | IRF3 Smartpool, IRF-1, IRF-2, | 5 |
| | | | IRF-3, IRF-4 | |
| 3 | Plasmid | IRF3 | IRF3 Smartpool | 6 |
| 4 | Plasmid | IRF3 | IRF3 Smartpool, IRF-1, IRF-2, | 7 |
| | | | IRF-3, IRF-4 | |

### Materials and Methods

In each experiment, primary human T cells were nucleofected and plated for incubation as described in Example 1. At day 3 post-nucleofection, cells numbers and viabilities were assessed using the Countess II FL (Life Technologies). Remaining cells were pipetted off the tissue culture plate and added to 15 ml conical tubes. To assess the total number and the percentage of cells that were CD3⁻ or CD3⁻/CAR⁺, 1×10⁵ cells/condition were then transferred to a 96-well plate. The plate was spun at 1300 RPM for 4 minutes and supernatants were decanted. Cells were re-suspended in 100 ul FACS buffer (PBS with 0.2% bovine serum albumin and 2 mM EDTA) and spun down to wash. After washing, 100 ul of antibody staining cocktail comprised of the following antibodies was added to each well:

CD3 BV785 Biolegend Catalog #317330, Clone OKT3

CD34 PE Invitrogen Catalog # MA1-10205, Clone QBEnd-10

Cells were re-suspended covered, and allowed to incubate for 15 minutes at room temperature. The plate was then spun down as above and supernatants were decanted. Cells were washed twice with 100 ul FACS buffer/wash and spun by centrifugation. For flow cytometry, cells were re-suspended in 200 ul FACS buffer and transferred to FACS tubes containing 200 ul pre-aliquoted FACS buffer (total volume 400 ul). Cells were run and data was collected using a BD Fortessa flow cytometer utilizing FACS Diva software. After flow staining, remaining cells were re-suspended in fresh T cell media supplemented with IL-2 at a concentration of 1×10⁶ viable cells/ml and plated in a new sterile 48-well plate, which was placed in a 37° C. incubator for 48-72 hours.

At day 5-6 post-nucleofection, cell numbers and viabilities were assessed using the Countess II FL (Life Technologies). Cells were then transferred to 15 ml conical tubes, with 1×10⁵ cells/condition aliquoted to a 96-well plate and stained with the same antibody cocktail utilized at day 3. Acquired flow cytometric data was analyzed using FlowJo software.

To calculate edited cell numbers/condition, the following equation was used:

Total viable cells×Frequency of CD3 negative (CD3⁻) cells as determined by flow cytometric analysis

To calculate CAR T cell number/condition, the following equation was used:

Total viable cells×Frequency of CD3⁻ CD34⁺ cells as determined by flow cytometric analysis.

### Results

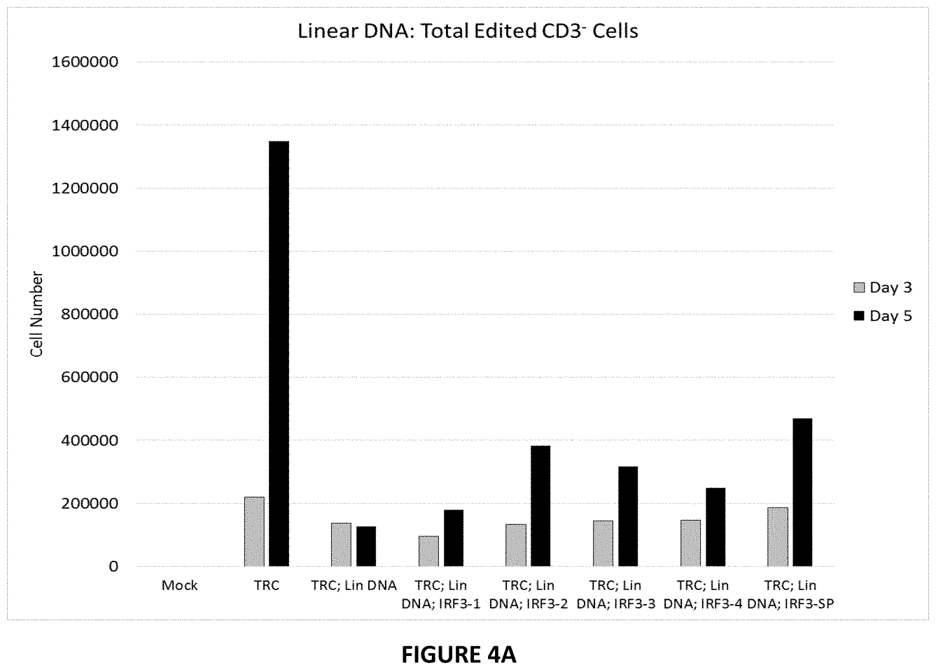

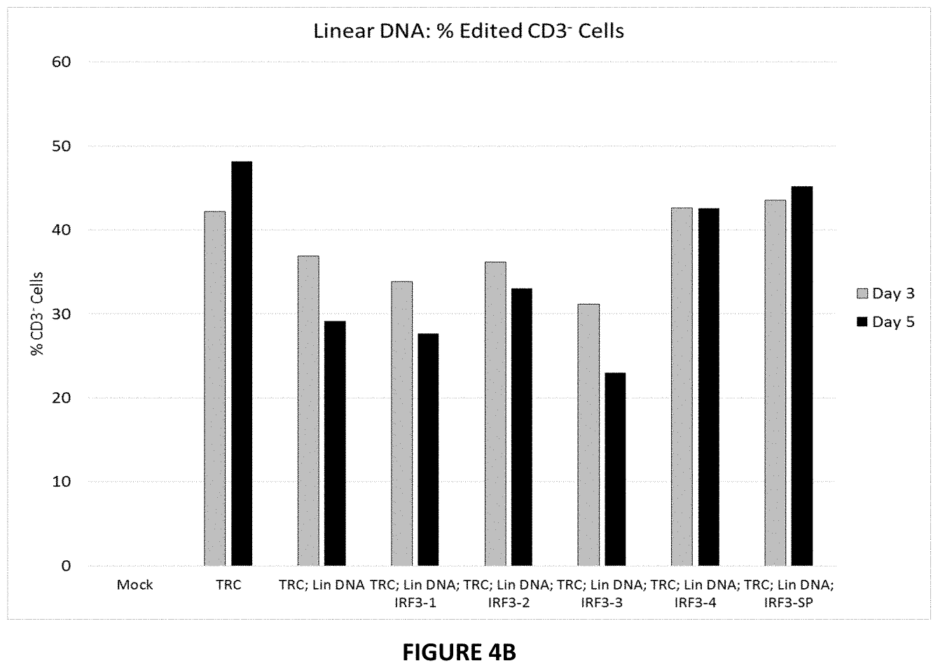

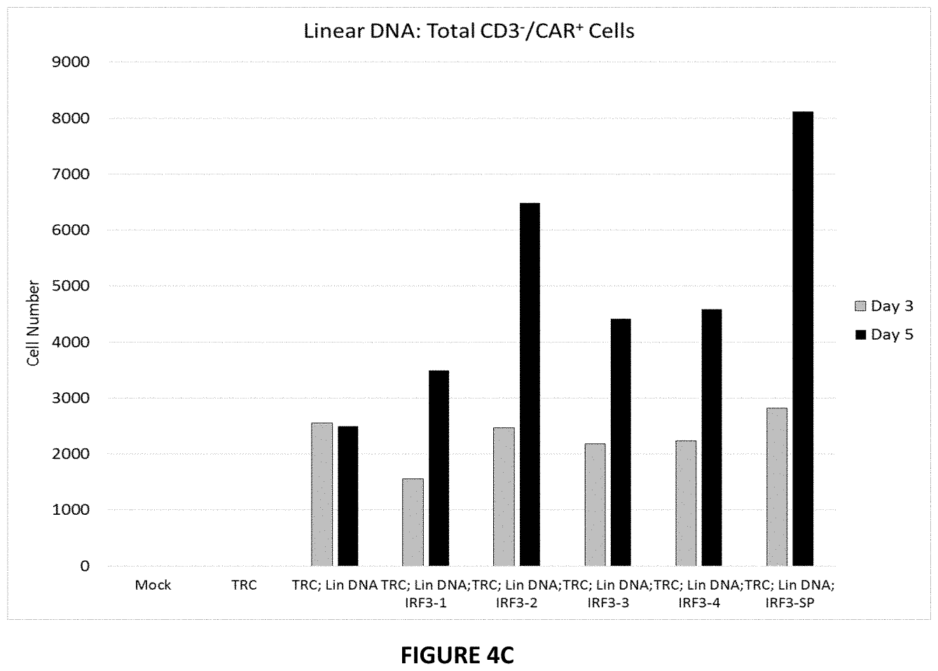

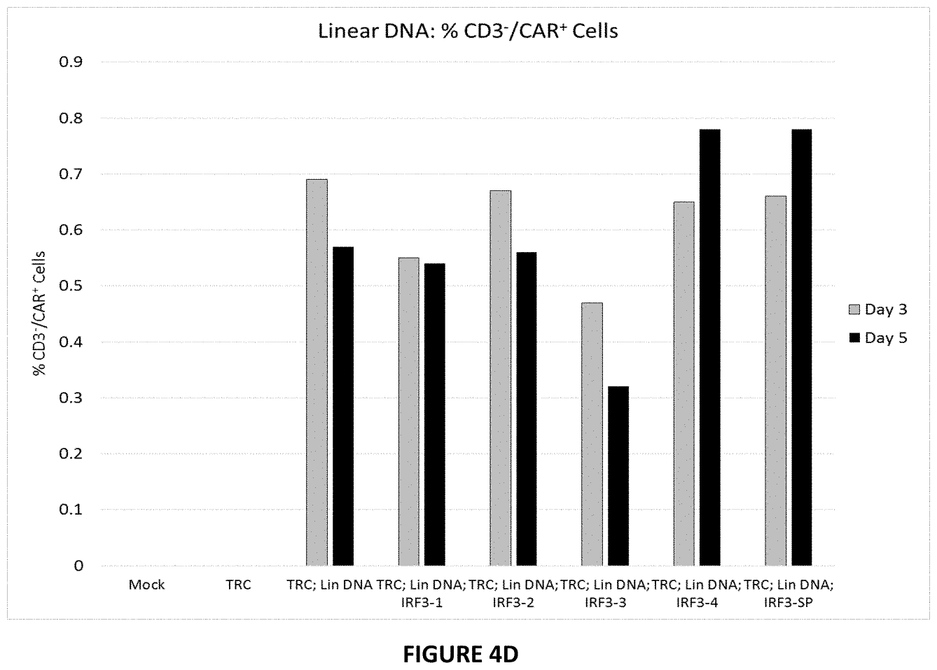

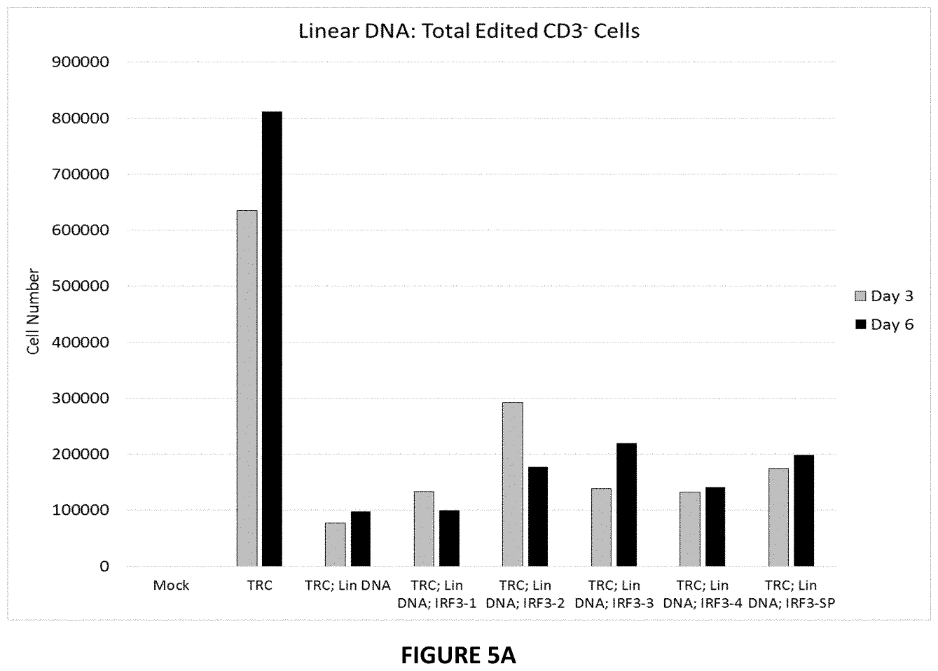

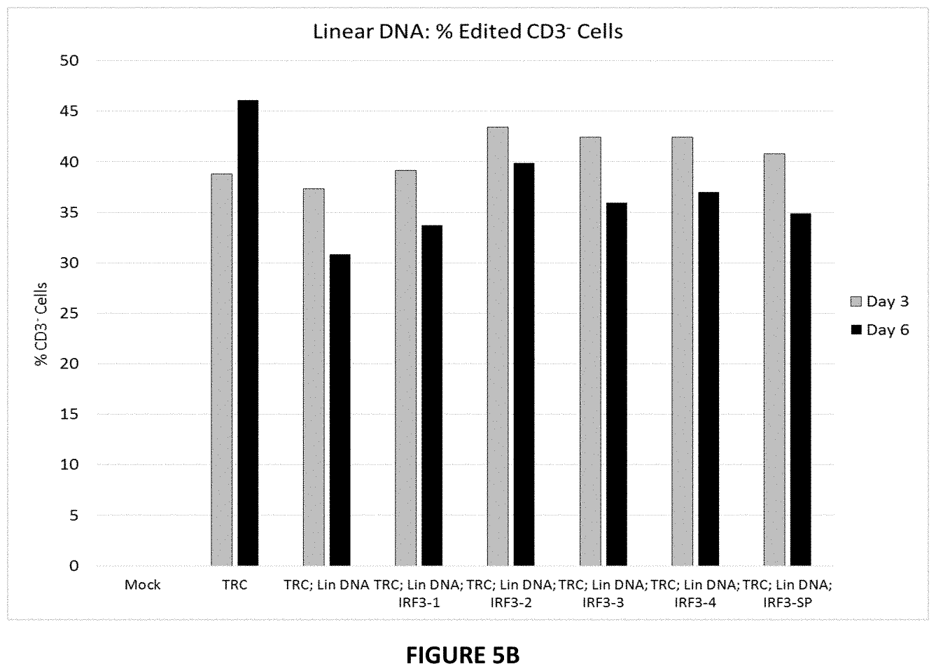

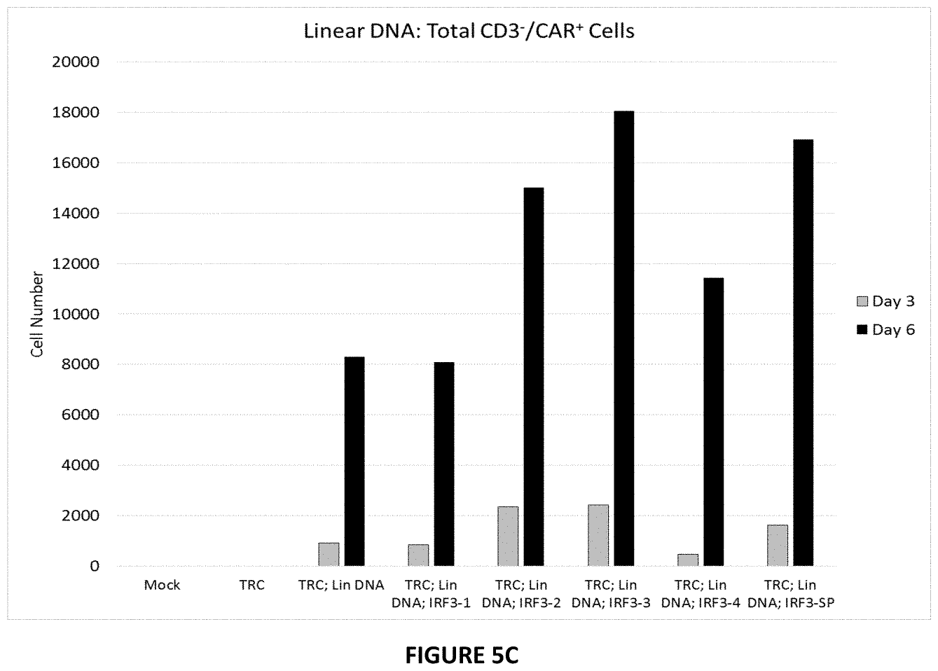

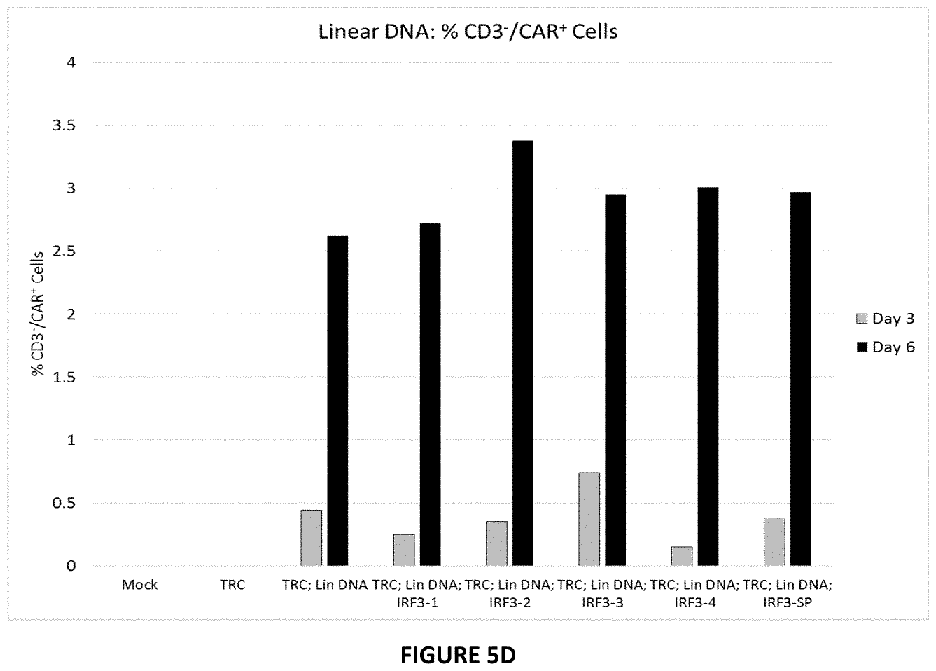

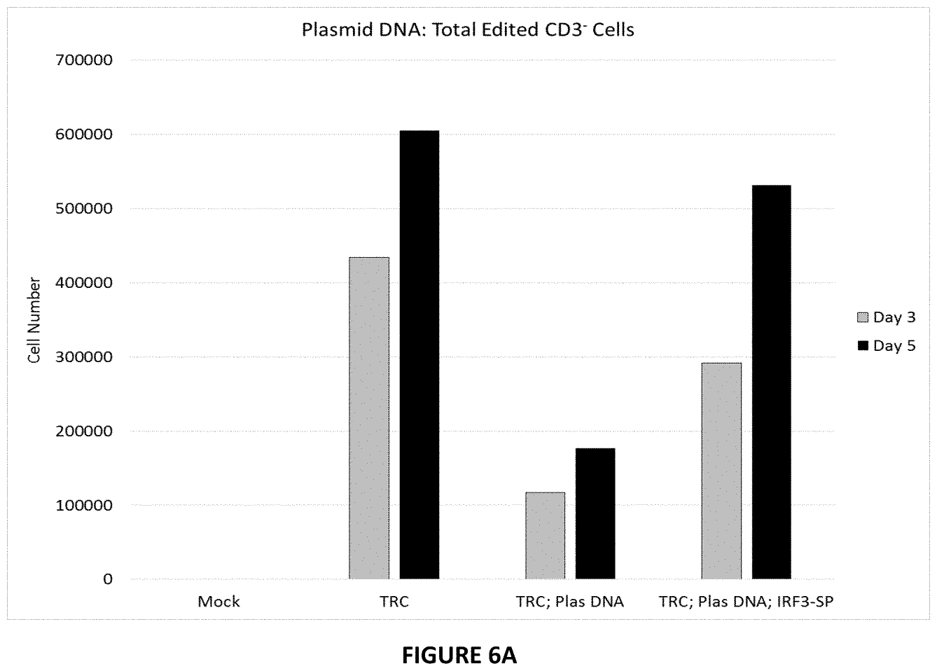

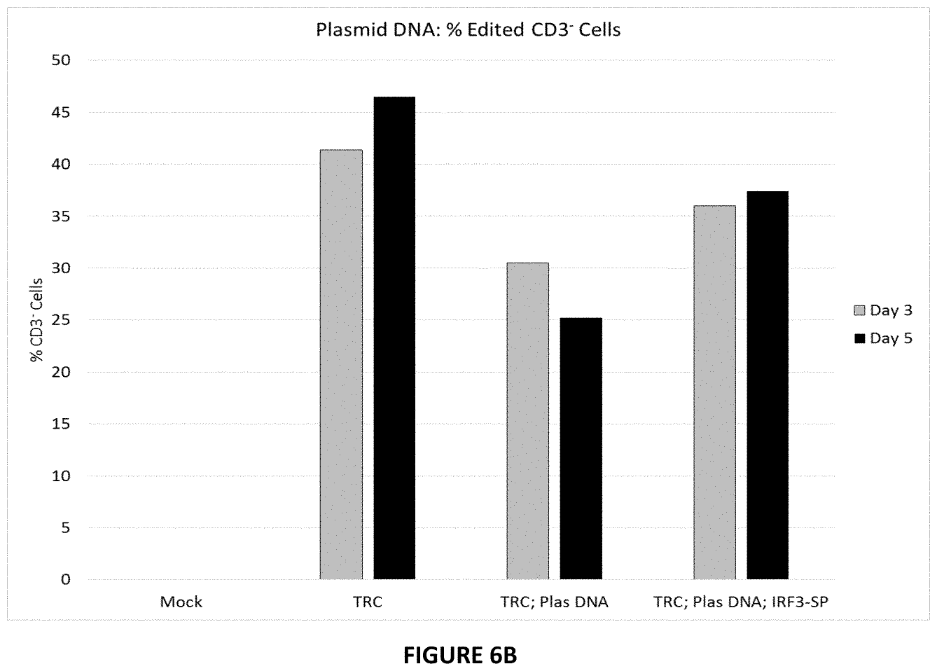

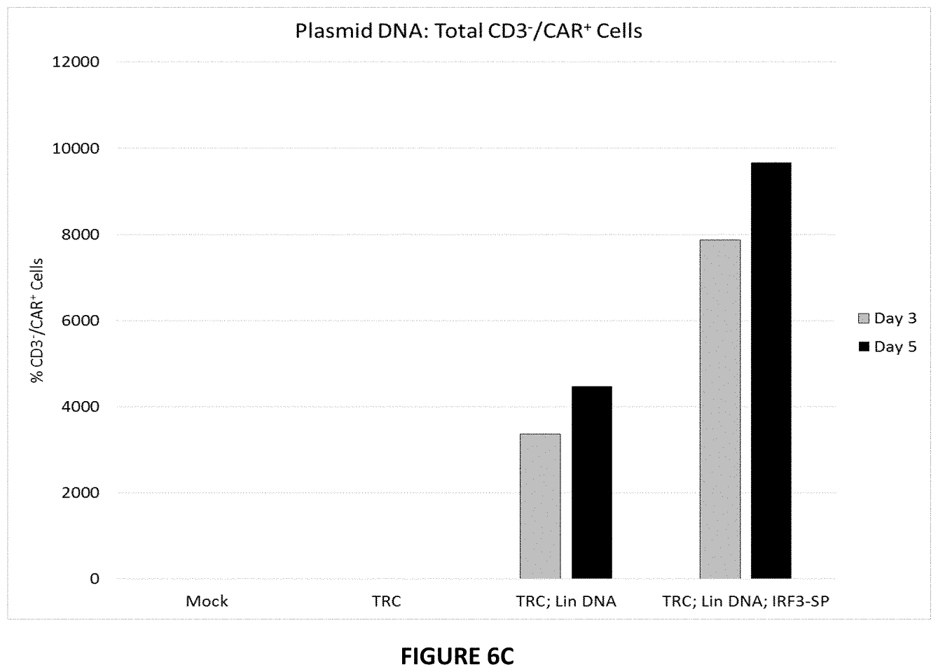

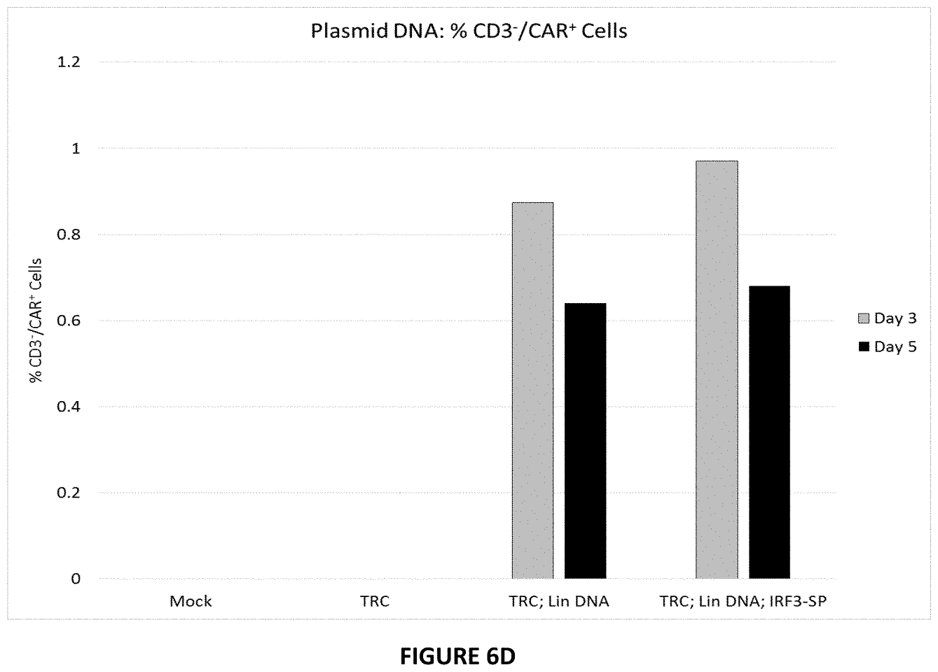

Nucleofection of T cells with linearized DNA and mRNA resulted in a marked reduction in edited cell number at various time points compared to mRNA-only nucleofected cells alone. Specifically, edited cells numbers were reduced 38-88% compared to controls at day 3 post-nucleofection (FIGS. 4A and 5A). This trend was maintained at both day 5 (90% reduction) (FIG. 4A) and 6 (88% reduction) (FIG. 5A) post-nucleofection and correlated with decreases in total cell numbers as seen in example 1. Importantly, addition of IRF3 smartpool siRNA prior to nucleofection increased edited cell numbers by 37% or more in duplicate experiments at day 3 (FIGS. 4A and 5A) and resulted in over a doubling in edited cell number at day 5 (FIG. 4A) and 6 (FIG. 5A) respectively compared to no siRNA control cells.

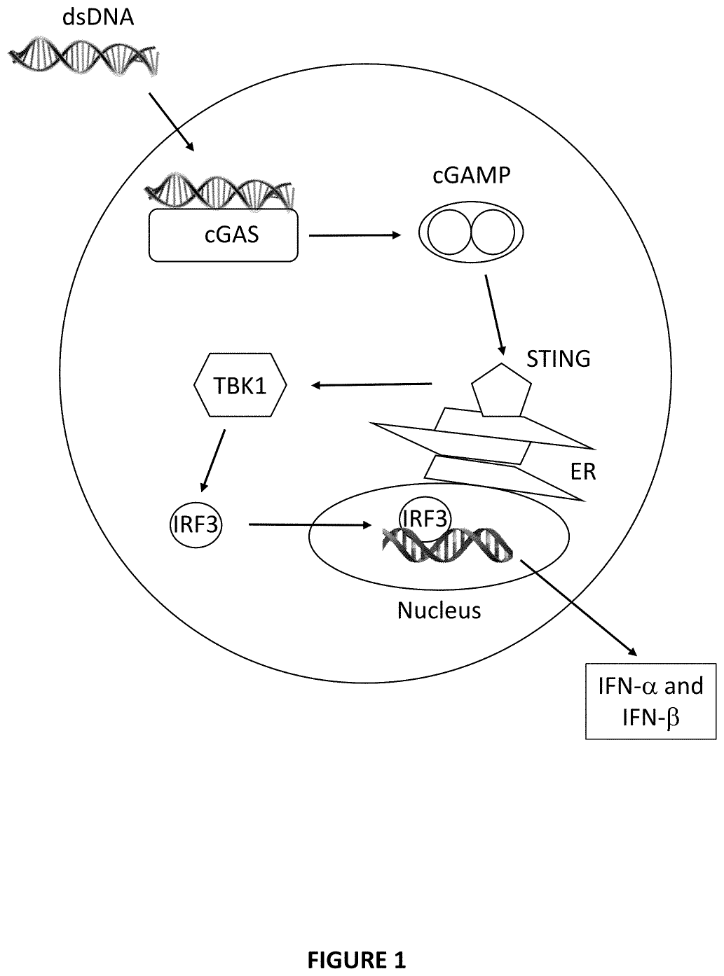

By comparison, addition of individual instead of smartpool IRF3 siRNA had variable effects on edited T cell frequency. At day 3, addition of IRF3-2 siRNA had the most drastic effect on edited T cell number in one experiment, resulting in a 3.8-fold increase in edited T cells compared to linear DNA and mRNA nucleofected control cells (FIG. 5A). Furthermore, IRF3-2 and IRF3-3 had similarly beneficial effects to IRF3 smartpool at day 5 and 6 post-nucleofection, with fold increases of at least 2.5-fold (day 5) (FIG. 4A) and 1.8-fold (day 6) (FIG. 5A) seen above no siRNA control conditions at these time points. This increase in total edited cell number correlated with a higher frequency of edited T cells in IRF3-2 or IRF3-3 siRNA conditions in one experiment (FIG. 5B).

In addition to both total and edited T cell numbers, the frequency and number of CAR T cells was also analyzed. At day 3 post-nucleofection, total CAR T cell numbers in linear DNA and mRNA co-nucleofected cells was 3×10⁴ or fewer in duplicate experiments (FIGS. 4C and 5C). Addition of IRF3 smartpool, IRF3-2, or IRF3-3 siRNA increased CAR T cell numbers 1.80-2.67-fold higher compared to no siRNA controls at this early time point (FIG. 5C). Moreover, CAR T cell numbers were also increased at day 5 (1.77-3.25-fold) (FIG. 4C) and day 6 (1.81-2.18-fold) (FIG. 5C) compared to control conditions. Interestingly, CAR T cell frequencies were below 1% of total viable cells at day 3 post-nucleofection in the presence of absence of any IRF3 siRNA derivative (FIGS. 4D and 5D). Additionally, only a minor increase in CAR T cell frequency was seen after addition of IRF3-3 in one experiment at this time point (FIG. 5D). Noteworthy, however, CAR T cell frequencies increased to over 2.5% of viable cells at day 6, with the highest frequency of CAR T cells in IRF3-2 siRNA nucleofected cells (FIG. 5D).

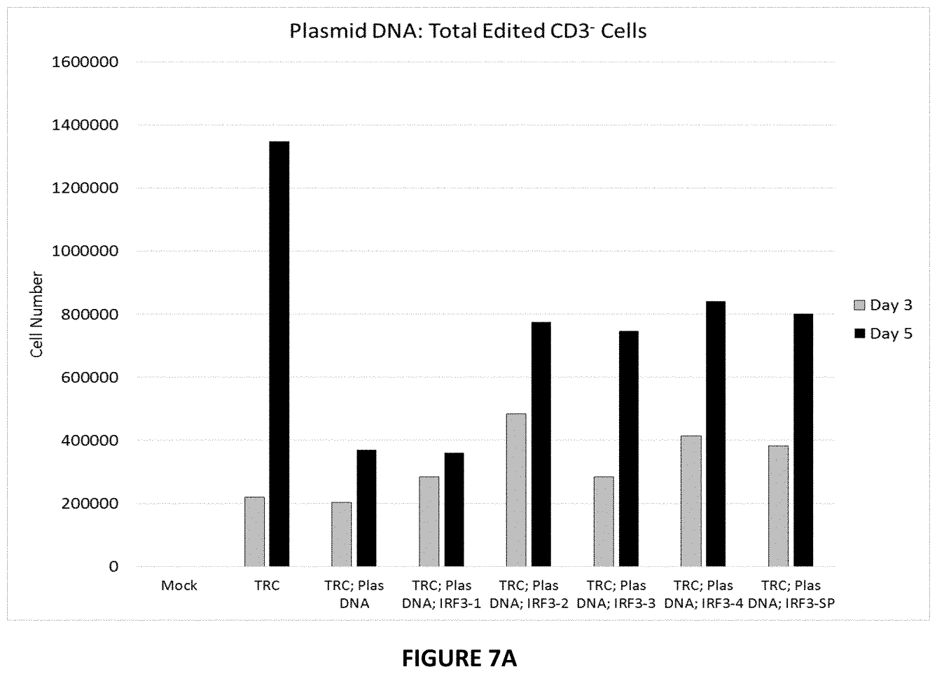

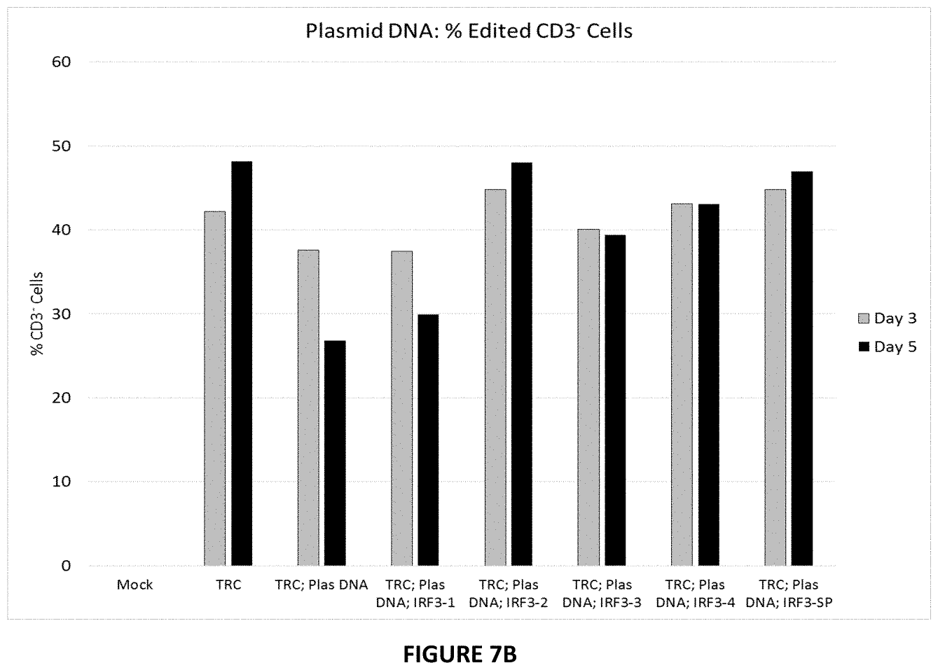

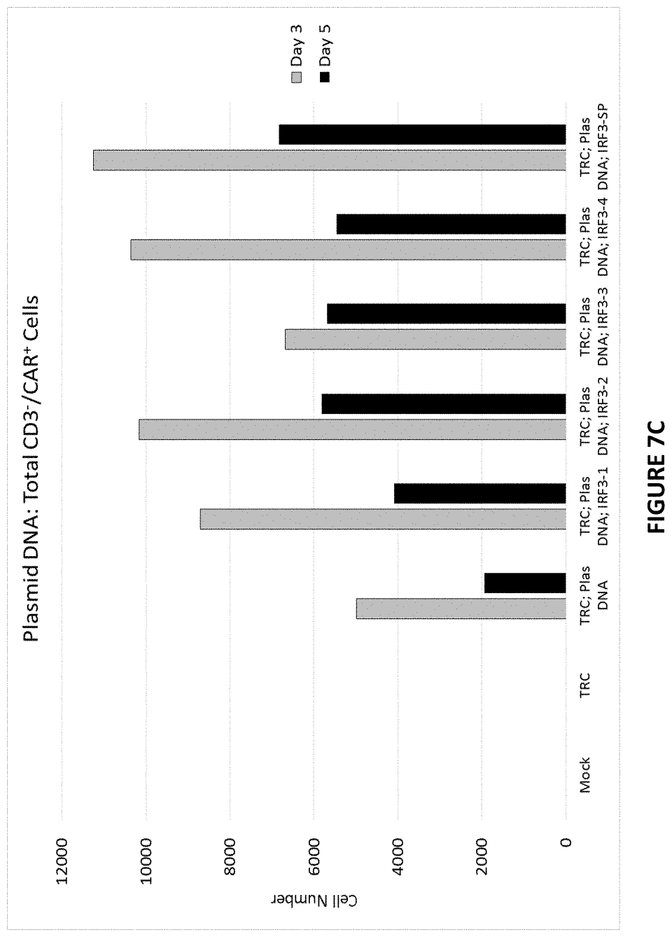

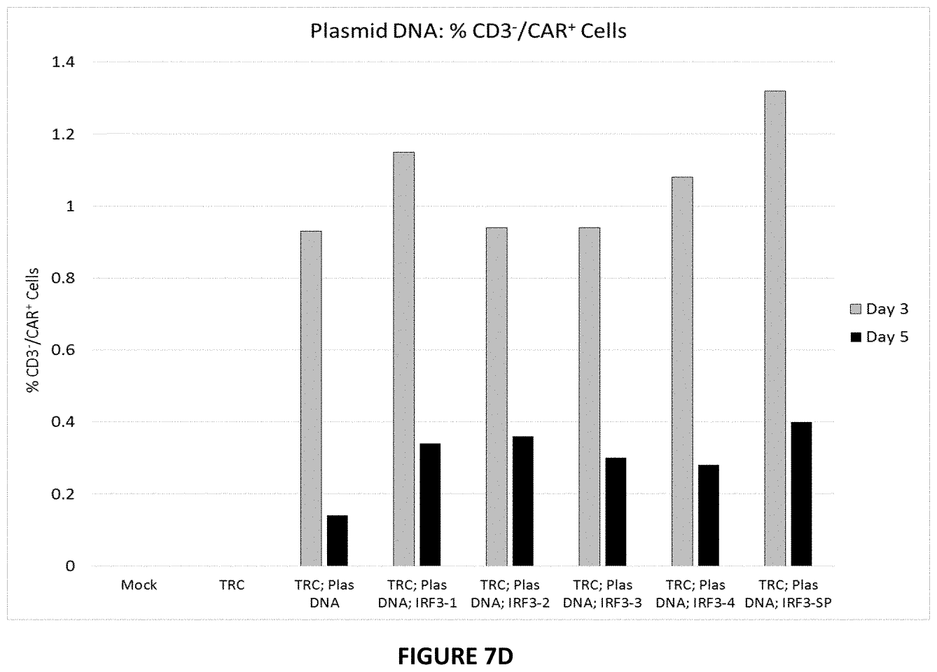

Similar to linear DNA, co-nucleofection of T cells with plasmid DNA and mRNA caused a sharp reduction, up to 73%, in edited cells compared to mRNA-only nucleofected cells (FIGS. 6A and 7A). The loss in edited cells, however, was mitigated by the addition of IRF3 smartpool siRNA, producing increases of close to 2-fold or more at day 3 and 5 post-nucleofection in duplicate experiments (FIGS. 6A and 7A). Total higher numbers of edited cells associated with increased edited cell frequencies at day 3 and 5 in duplicate experiments (FIGS. 6B and 7B). Furthermore, this increase in edited cell number correlated with improved CAR T cell recovery after addition of IRF3 smartpool siRNA. Specifically, CAR T cell numbers were 125-134% higher at day 3 and 116-253% higher at day 5 post-nucleofection compared to no siRNA controls (FIGS. 6C and 7C), with CAR T cell frequencies showing an associated increase as well (FIGS. 6D and 7D). Interestingly, the frequency of CAR T cells was also reduced between day 3 and later time points despite increases in the quantity of CAR T cells (FIGS. 6D and 7D).

The use of individual IRF3, instead of smartpool, siRNA almost universally increased both edited and CAR T cell numbers at day 3 and day 5 post-nucleofection (FIGS. 7A and 7C). While the range of effects was wide, the largest growth was seen when IRF3-2 and IRF3-4 siRNA was utilized, which both showed close to 2-fold increases in edited and CAR T cells at both time points analyzed (FIGS. 7A and 7C). Importantly, the uptick in cells was similar to the effect seen when the IRF3 smartpool was used. Lastly, edited T cell, and to a lesser extent CAR T cell, frequencies were higher with either IRF3-2 or IRF3-4 variant compared to no siRNA controls (FIGS. 7B and 7D).

### Conclusions

Co-nucleofection of primary human T cells with mRNA and either linear or plasmid DNA results in substantial reductions in recovered edited and CAR T cells at various time points post-nucleofection. The use of IRF3 smartpool or the specific IRF3-2 variant siRNA during nucleofection drastically increased the number of both edited and CAR T cells. These improved cell numbers were associated with corresponding increases in cellular frequencies within the T cell pool as a whole. Taken in concert, this suggests that diminishing IRF3 protein levels in primary human T cells can increase gene-edited, and most importantly, CAR T cell recovery, after introduction of cytosolic DNA.

### Example 3

### Effect of STING Knockdown on Gene Editing and Insertion of an Exogenous Nucleic Acid by Homologous Recombination

### Purpose

The purpose of this experiment was to determine the effect of STING knockdown on gene editing and targeted insertion by homologous recombination in primary cells, particularly in primary human T cells.

Multiple experiments were performed to examine the effect of STING knockdown by siRNA on the same four different parameters described above in Example 2. Similarly, in each experiment, cells were tested with groups including (i) mock nucleofection, (ii) TRC nuclease only, (iii) TRC nuclease with plasmid or linear DNA, and (iv) TRC nuclease, plasmid or linear DNA, and siRNA. The STING siRNAs evaluated in each experiment are outlined in Table 4 below.

TABLE 4

| | DNA | | | |
|---|---|---|---|---|
| Experiment | Template | Target | siRNAs | Figure |
| 1 | Linear | STING | STING Smartpool, STING-1, | 8 |
| | | | STING-2, STING-3, STING-4 | |
| 2 | Linear | STING | STING-1 | 9 |
| 3 | Plasmid | STING | STING Smartpool, STING-1 | 10 |
| 4 | Plasmid | STING | STING-1 | 11 |

### Materials and Methods

In each experiment, primary human T cells were nucleofected and plated for incubation as described in Example 1. At day 3 post-nucleofection, cells numbers and viabilities were assessed using the Countess II FL (Life Technologies). Remaining cells were pipetted off the tissue culture plate and added to 15 ml conical tubes. To assess edited and CAR T cell frequency in cells nucleofected with or without STING siRNA, 1×10⁵ cells/condition were then transferred to a 96-well plate. The plate was spun at 1300 RPM for 4 minutes and supernatants were decanted. Cells were re-suspended in 100 ul FACS buffer and spun down to wash. After washing, 100 ul of antibody staining cocktail comprised of the following antibodies was added to each well:

CD3 BV785 Biolegend Catalog #317330, Clone OKT3

CD34 PE Invitrogen Catalog # MA1-10205, Clone QBEnd-10

Cells were re-suspended, covered, and allowed to incubate for 15 minutes at room temperature. The plate was then spun down as above and supernatants were decanted. Cells were washed twice with 100 ul FACS buffer/wash and spun by centrifugation. For flow cytometry, cells were re-suspended in 200 ul FACS buffer and transferred to FACS tubes containing 200 ul pre-aliquoted FACS buffer (total volume 400 ul). Cells were run and data was collected using a BD Fortessa flow cytometer utilizing FACS Diva software. After flow staining, remaining cells were re-suspended in fresh T cell media supplemented with IL-2 at a concentration of 1×10⁶ viable cells/ml and plated in a new sterile 48-well plate, which was placed in a 37° C. incubator for 48-72 hours.

At day 5 post-nucleofection, cell numbers and viabilities were assessed using the Countess II FL (Life Technologies). Cells were then transferred to 15 ml conical tubes, with 1×10⁵ cells/condition aliquoted to a 96-well plate and stained with the same antibody cocktail utilized at day 3. Acquired flow cytometric data was analyzed using FlowJo software.

To calculate edited cell numbers/condition, the following equation was used:

Total viable cells×Frequency of CD3 negative (CD3⁻) cells as determined by flow cytometric analysis

To calculate CAR T cell number/condition, the following equation was used:

Total viable cells×Frequency of CD3-CD34⁺ cells as determined by flow cytometric analysis.

### Results

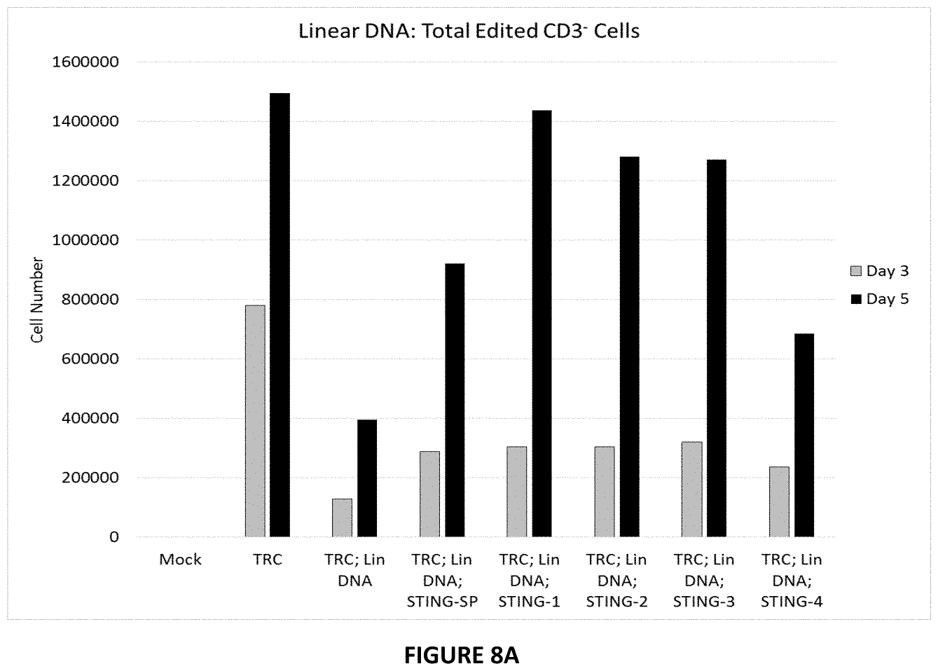

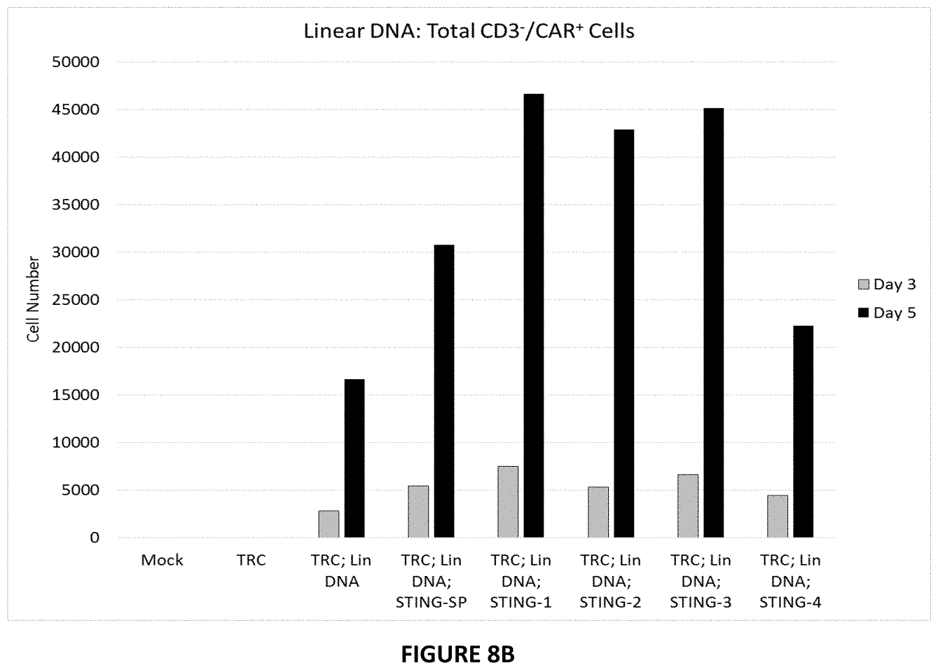

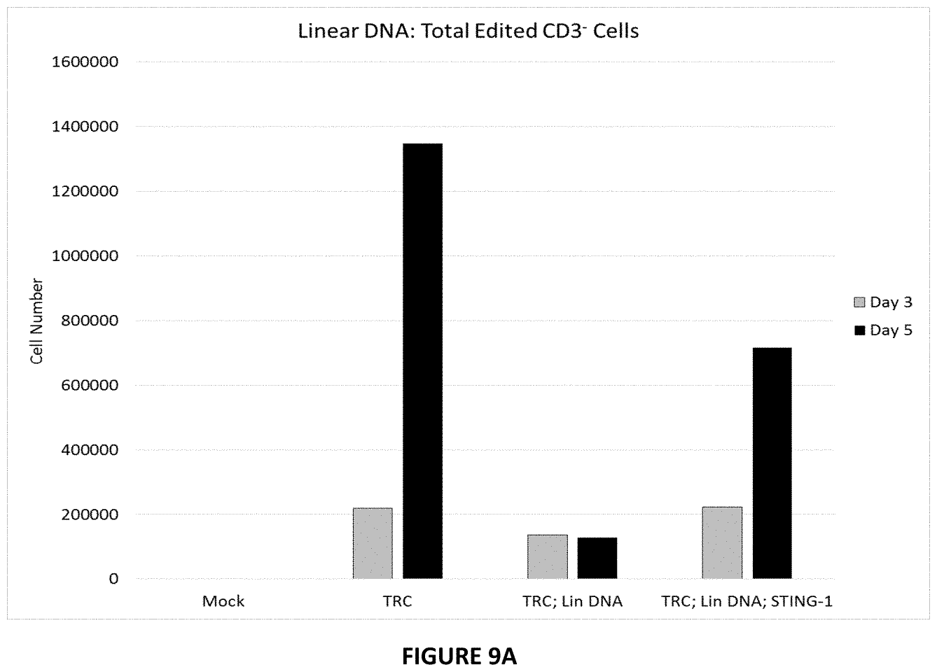

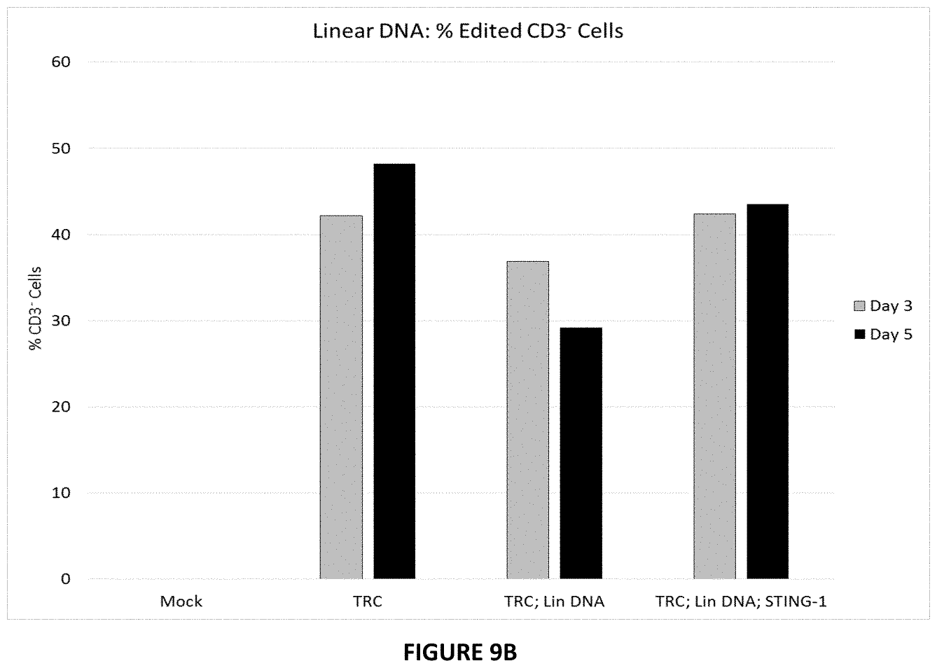

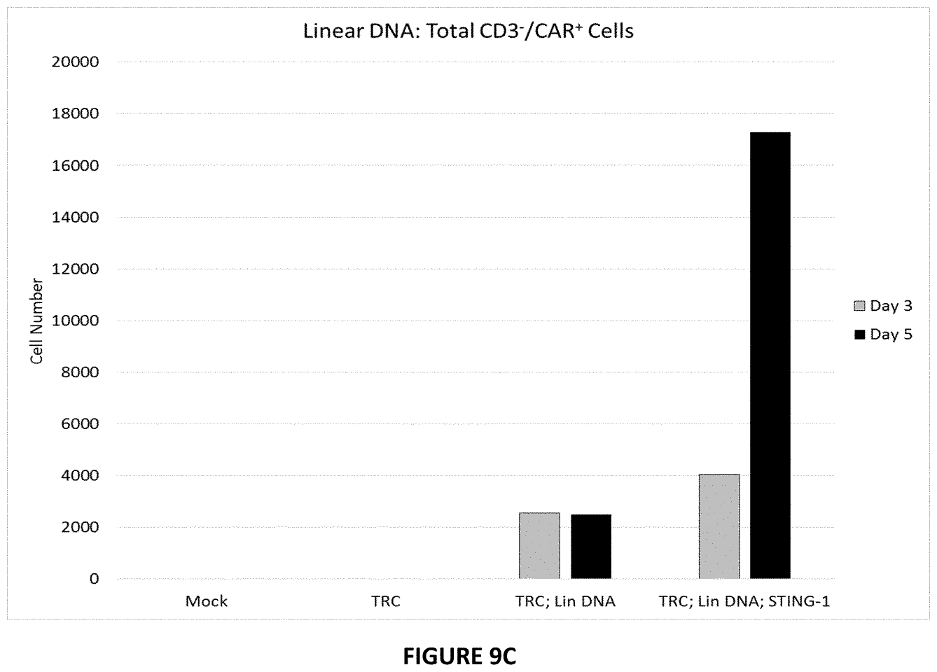

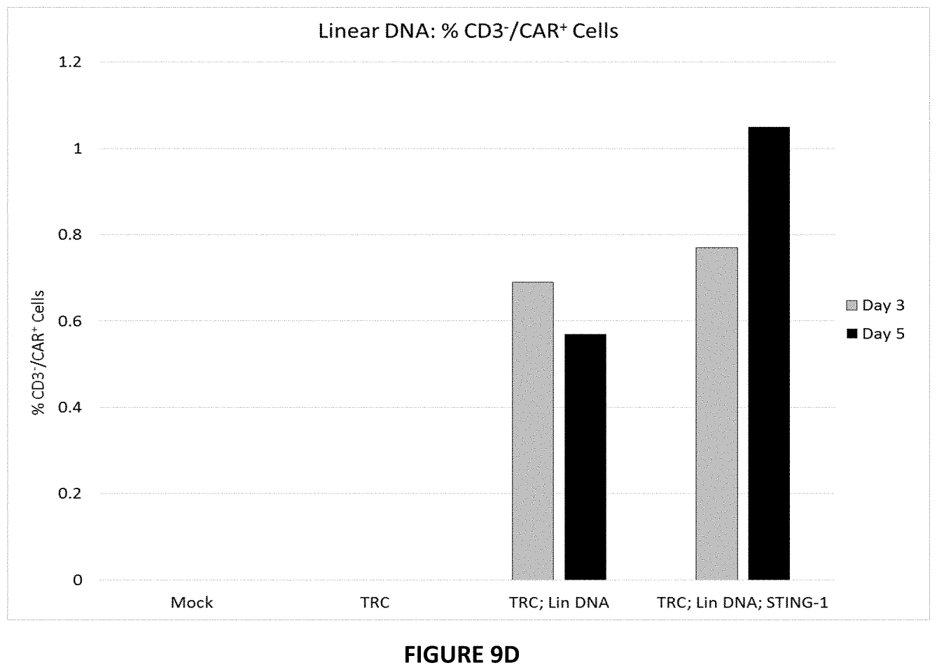

STING is a cytosolic protein that signals upstream of IRF3 and is involved in the recognition of foreign DNA as part of the cGAS pathway. To support the contribution of this pathway in increasing edited and CAR T cell number after nucleofection of DNA and mRNA by reducing cytotoxicity, STING smartpool or individual siRNA was added to nucleofection mixtures. As in previous experiments, nucleofection of human T cells with linear DNA and mRNA alone resulted in significant decreases in edited T cell numbers at various time points post-nucleofection compared to controls (FIGS. 8A and 9A). Encouragingly, however, the addition of STING smartpool or individual variant siRNA increased edited T cell recovery by 1.63-2.48-fold at day 3 post-nucleofection and 1.73-5.62-fold at day 5 post-nucleofection compared to no siRNA controls, with STING-1 variant siRNA showing the greatest effect on edited T cells recovered (FIGS. 8A and 9A). This increase in total edited T cells was reflected in the amount of CAR T cells recovered, with up to a 6.9-fold increase in CAR T cells from STING-1 conucleofected cells at day 5 compared to mRNA and DNA control T cells (FIG. 9C). Of note, addition of STING siRNA, whether smartpool or other variants, universally had positive effects on CAR T cell recovery at both day 3 and 5 (FIG. 8B).

As previously seen, co-nucleofection of T cells with linear DNA and mRNA results in a decreased frequency of edited T cells compared to mRNA nucleofected controls, with edited T cell frequencies down 5% and 19% at day 3 and 5 respectively (FIG. 9B). Importantly, this loss in edited T cell frequency was mitigated by the addition of STING-1 siRNA, with frequencies almost back to equivalent levels as seen in mRNA controls (FIG. 9B). As a result, CAR T cell frequencies in cells conucleofected with STING-1 siRNA were almost 2-fold higher at day 5 post-nucleofection compared to no siRNA controls (FIG. 9D).

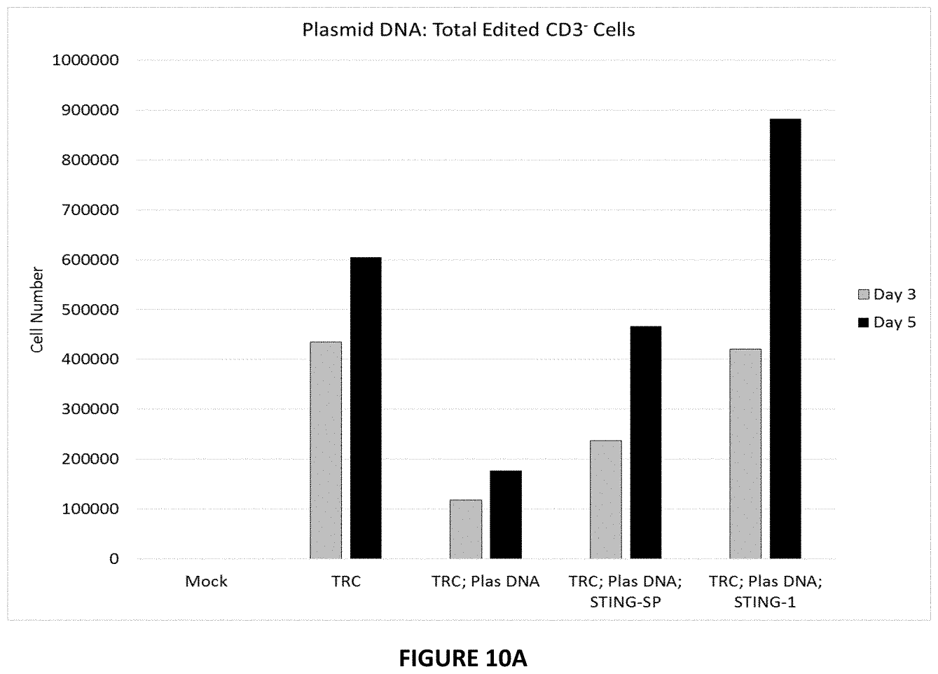

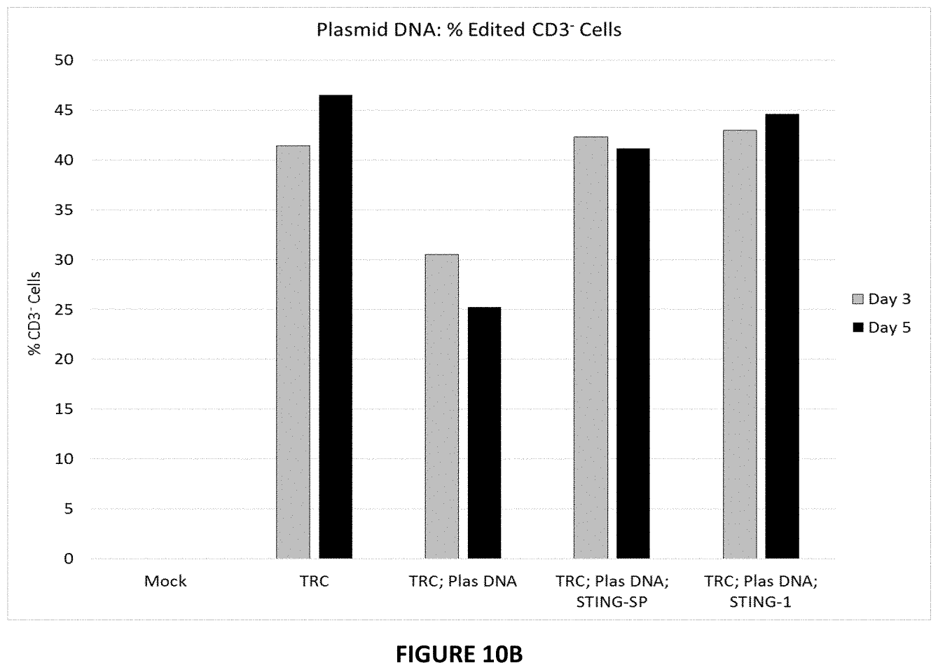

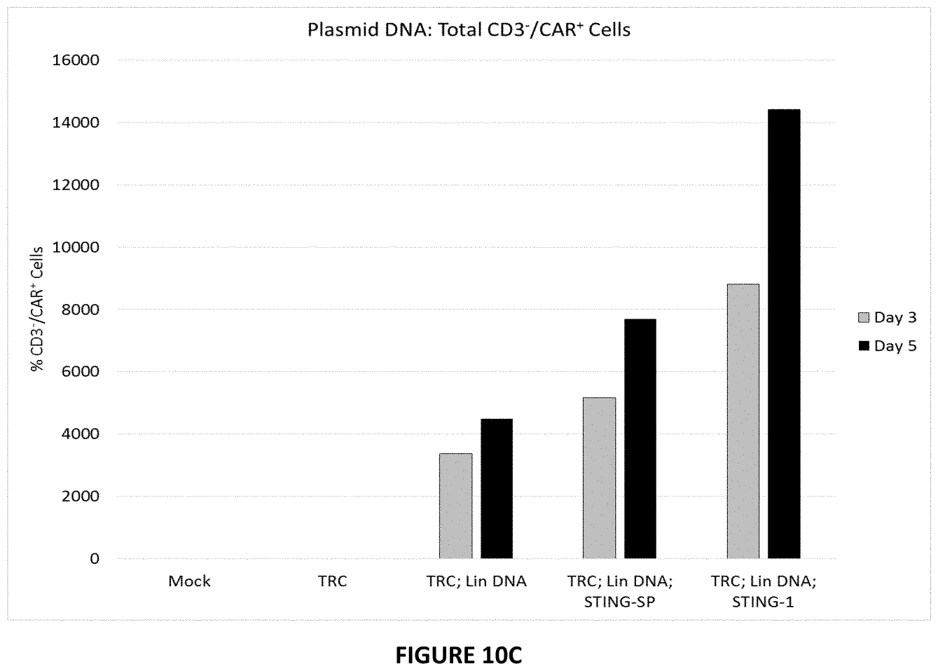

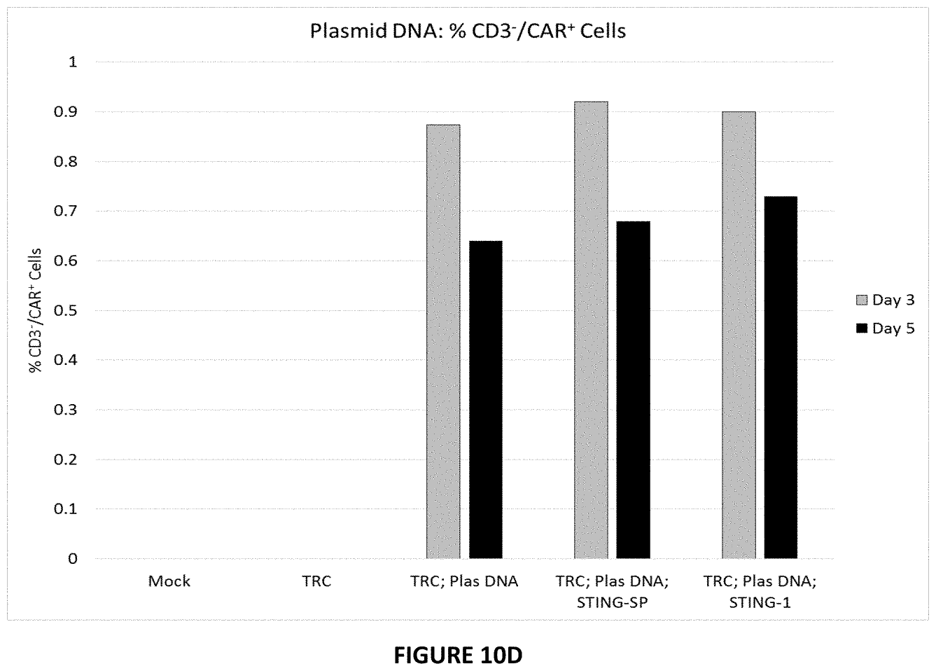

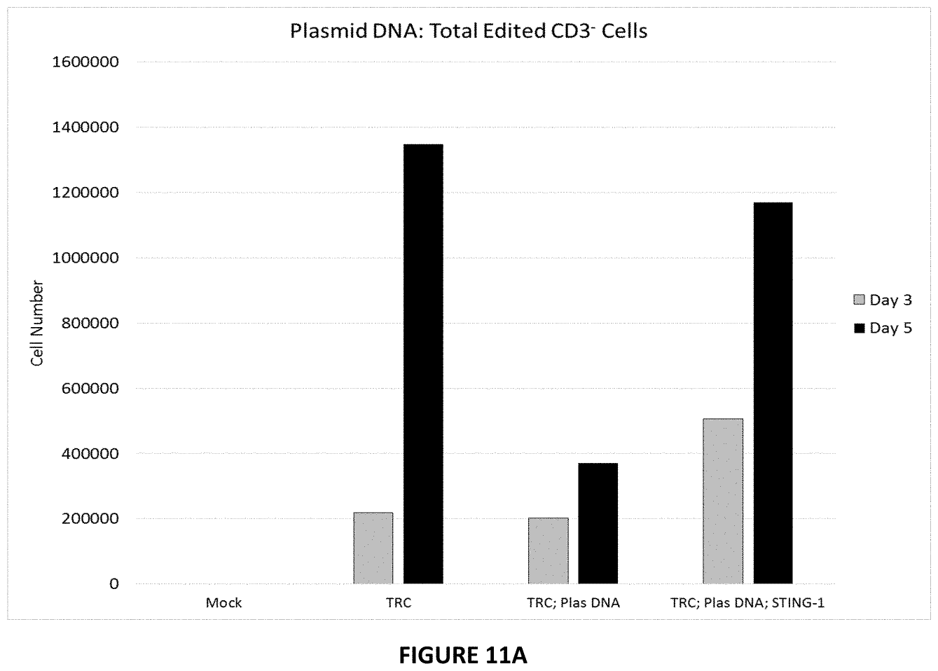

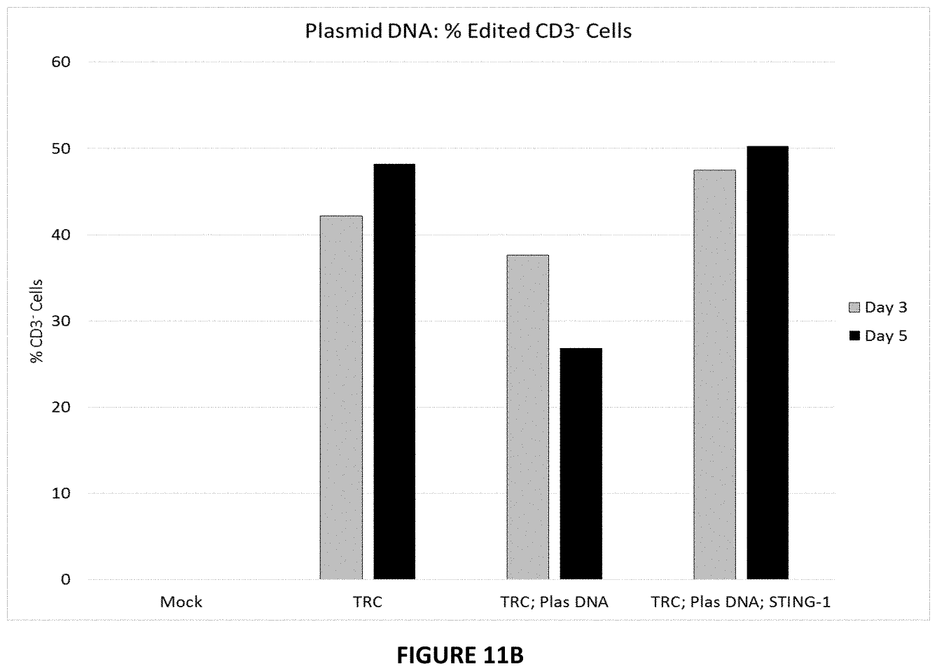

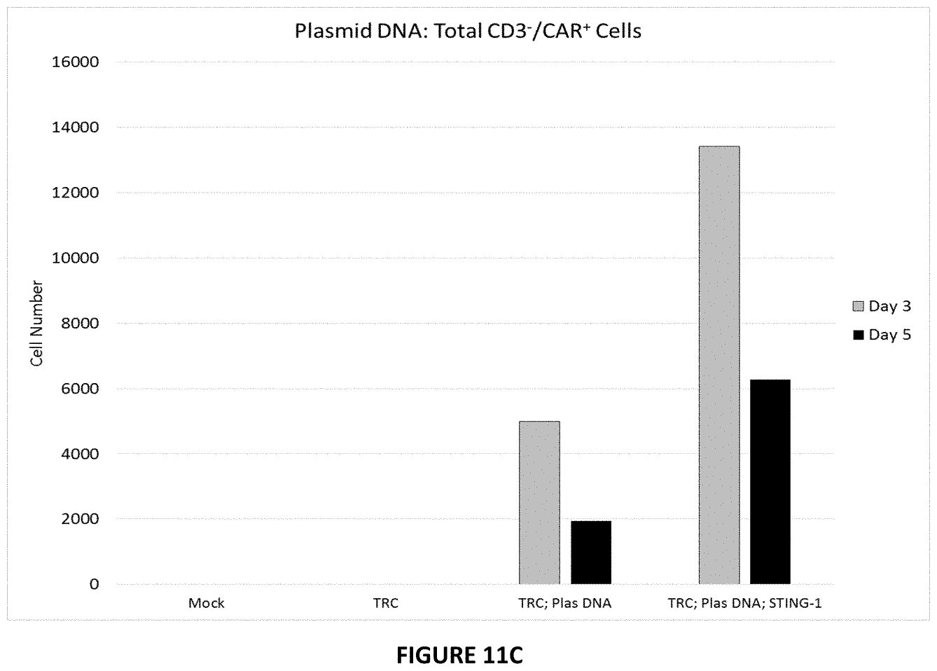

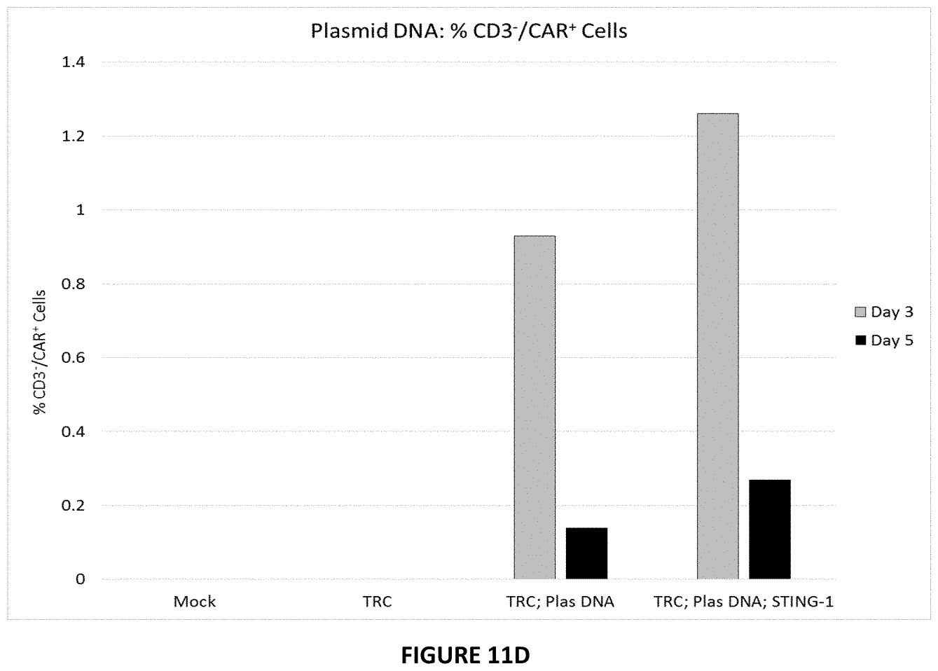

In the context of plasmid DNA, both STING smartpool and STING-1 variant siRNA increased edited cell numbers compared to control T cells (FIGS. 10A and 11A). Of note, the addition of STING smartpool siRNA increased edited cells over 2-fold at both day 3 and 5 (FIG. 10A). Similar to linear DNA, however, STING-1 variant siRNA had a more drastic effect, with edited T cells increasing 2.5-3.6-fold and 3.2-5.0-fold at day 3 and day 5 post-nucleofection (FIGS. 10A and 11A). This increase in total edited T cells witnessed with the addition of STING smartpool or STING-1 siRNA correlated with an increase in edited cell frequency at both time points analyzed. Specifically, a 26-39% increase in edited cell frequency was seen at day 3, while a 63-87% increase in edited cell frequency was seen at day 5, over linear DNA and mRNA nucleofected cells alone (FIGS. 10B and 11B).

Potential alterations in CAR T cell frequencies and numbers were also analyzed in plasmid DNA and mRNA nucleofected cells with or without STING siRNA. Of interest, both STING smartpool and STING-1 variant siRNA increased CAR T cell numbers at day 3 and day 5 (FIGS. 10C and 11C). In duplicate experiments, STING-1 resulted in a 2.6-fold increase at day 3 and a 3.2-fold increase at day 5 post-nucleofection (FIGS. 10C and 11C). By comparison STING smartpool had a notable, but more modest, effect on CAR T cell number with a 1.5-1.7-fold increase seen compared to no siRNA controls at the time points analyzed (FIG. 10C). Furthermore, although CAR T cell frequencies are lower with plasmid compared to linear DNA, the inclusion of STING-1 or STING smartpool siRNA resulted in a consistent increase in CAR T cell frequency (FIGS. 10D and 11D), with almost a 2-fold increase seen at day 5 in one representative experiment (FIG. 11D).

### Conclusions

The addition of siRNA specific for the cytosolic protein STING increased total edited and CAR T cell numbers and frequencies in cells co-nucleofected with mRNA and DNA compared to no siRNA controls. As a result, this implies that STING, together with its downstream mediator IRF3, play intrinsic roles during cellular responses to cytosolic foreign DNA. As such, blockade of this STING-IRF3 pathway through the use of siRNA provides a novel mechanism by which to reduce cytotoxicity associated with the introduction of cytosolic DNA and thus improve the formation and sustainability of genetically modified primary human T cells.

### Example 4

### Effect of IRF3 and STING Knockdown on Primary T Cell Phenotype

### Purpose

The purpose of this experiment was to determine the effect of IRF3 or STING knockdown on the phenotype of primary human T cells. Studies have shown that CAR T cells that have a higher frequency of naïve/central memory T cells are less exhausted and, as a result, form a longer lasting CAR T cell product.

### Materials and Methods

Primary human T cells were nucleofected and plated for incubation as described in Example 1. At day 5 post-nucleofection, cells numbers and viabilities were assessed using the Countess II FL (Life Technologies). Remaining cells were pipetted off the tissue culture plate and added to 15 ml conical tubes. To enrich for edited T cells, the remaining CD3⁺ T cells were magnetically separated using the Stemcell Easysep release human CD3 positive selection kit and the Easyplate EasySep magnet as per the manufacturers recommendations from each condition. Post-separation, cell numbers and viabilities were re-assessed using the Countess II FL (Life technologies). CD3-depleted T cell fractions were then cultured in media supplemented with IL-15 and IL-21 (Gibco) at a concentration of 1×10⁶ cells/ml and placed in the incubator.

At day 11 post-nucleofection cells were transferred to 15 ml conicals, spun down, and cell numbers and viabilities were recorded as above. 2.5×10⁵ cells/condition were transferred to a 96-well round bottom plate and the plate was spun at 1300 RPM for 4 minutes, followed by removal of the supernatants. Cells were re-suspended in 100 ul FACS buffer and spun down to wash. To assess the phenotype of CAR T cells, 100 ul of antibody staining cocktail comprised of the following antibodies was added to each well:

CD3 BV785 Biolegend Catalog #317330, Clone OKT3

CD34 PE Invitrogen Catalog # MA1-10205, Clone QBEnd-10

CD4 APC Biolegend Catalog #317416, Clone OKT4

CD8a BV421 Biolegend Catalog #301036, Clone RPA-T8

CD62L BB515 BD Catalog #564742, Clone DREG-56

CD45RO PeCy7 Biolegend Catalog #364230, Clone UCHL1

Cells were re-suspended, covered, and allowed to incubate for 15 minutes at room temperature. The plate was then spun down as above and supernatants were decanted. Cells were washed twice with 100 ul FACS buffer/wash and spun by centrifugation. For flow cytometry, cells were re-suspended in 200 ul FACS buffer and transferred to FACS tubes containing 200 ul pre-aliquoted FACS buffer (total volume 400 ul). Cells were run and data was collected using a BD Fortessa flow cytometer utilizing FACS Diva software.

For analysis, cells were first gated on live populations based on forward scatter and side scatter profiles. CAR T cells were then gated for phenotypic analysis based on CD3 CD34⁺ expression, which was subsequently applied to a bivariant graph depicting CD62L expression on the y-axis and CD45RO expression on the x-axis. This bivariant graph was divided into four quadrants. Moving clockwise, cells in the top left (CD62L⁺ CD45RO⁻) are loosely classified as naïve, followed by central memory T cells (CD62L⁺ CD45RO⁺), transitional memory (CD62L⁻ CD45RO⁺), and finally effector memory/EMRA (CD62L⁻ CD45RO⁻).

### Results

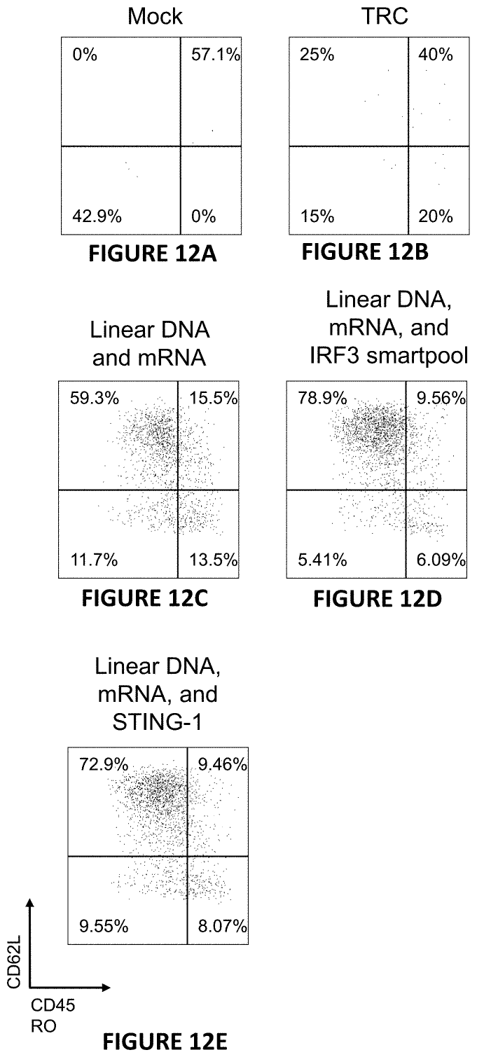

To assess the phenotype of CAR T cells that were nucleofected with linear DNA alone, or with linear DNA and either STING or IRF3 smartpool siRNA, CD3-CD34⁺ T cells were gated on during flow cytometric analysis. CAR T cells were then analyzed on a bivariant graph with CD62L and CD45RO. As expected, few cells from mock nucleofected or T cells nucleofected with mRNA only fell within the pre-defined CAR T cell gate as neither condition was nucleofected with DNA expressing our CAR gene (FIG. 12). Any cells that were collected from these conditions in that gate are representative of non-specific background fluorescence during flow cytometry data collection.

In CAR T cells nucleofected with linear DNA in the absence of siRNA, 59.3% at day 11 post-nucleofection were naïve, with an additional 15.5% classified as central memory T cells (FIG. 12). Interestingly, in CAR T cells nucleofected with either IRF3 or STING smartpool siRNA, there was a notable rise in naïve frequencies at this time point. Specifically, the frequency of naïve CAR T cells rose almost 20% in cells nucleofected with IRF3 smartpool siRNA to 78.9%, with a compensatory drop in effector memory/EMRA (5.41%), transitional memory (6.09%) and central memory (9.56%) CAR T cells (FIG. 12). By comparison, co-nucleofection with STING smartpool siRNA resulted in a similar increase in naïve T cells up to 72.9%, resulting in close to a 14% improvement compared to no siRNA controls (FIG. 12). As with IRF3, this increase in naïve CAR T cells correlated with a drop in the other three T cell populations.

### Conclusions

Co-nucleofection of primary T cells with linear DNA and either IRF3 or STING smartpool siRNA resulted in a CAR T cell population comprised of a higher frequency of naïve cells compared to no siRNA controls. This rise in naïve CAR T cells would produce a less exhausted CAR T cell product that would functionally be longer lasting compared to one comprised of more differentiated cells.

### Example 5

### Effect of IRF3 and STING Knockdown on Primary T Cell CD4/CD8 Distribution

### Purpose

The purpose of this experiment was to determine the effect of IRF3 or STING knockdown on the CD4/CD8 distribution of primary human T cells. T cells in general can be subdivided into CD4 and CD8 populations. CD8 T cells, otherwise known as cytotoxic T lymphocytes, as generally defined as the T cells that induce cytotoxicity in target populations. For the functionality of these cells, however, CD8 T cells need support from other cells types that secrete proteins such as pro-survival cytokines like IL-2. CD4, or T helper cells, are one pivotal population that provides this help to the cytotoxic CD8. Maintaining a higher population of CD4 T cells with a CAR T cell product, therefore, could potentially result in a longer lasting and more potent population.

### Methods and Materials

Primary human T cells were nucleofected and plated for incubation as described in Example 1. At day 5 post-nucleofection, cells numbers and viabilities were assessed using the Countess II FL (Life Technologies). Remaining cells were pipetted off the tissue culture plate and added to 15 ml conical tubes. To enrich for edited T cells, the remaining CD3⁺ T cells were magnetically separated using the Stemcell Easysep release human CD3 positive selection kit and the Easyplate EasySep magnet as per the manufacturers recommendations from each condition. Post-separation, cell numbers and viabilities were re-assessed using the Countess II FL (Life technologies). CD3-depleted T cell fractions were then cultured in media supplemented with IL-15 and IL-21 (Gibco) at a concentration of 1×10⁶ cells/ml and placed in the incubator.

At day 11 post-nucleofection cells were transferred to 15 ml conicals, spun down, and cell numbers and viabilities were recorded as above. 2.5×10⁵ cells/condition were transferred to a 96-well round bottom plate and the plate was spun at 1300 RPM for 4 minutes, followed by removal of the supernatants. Cells were re-suspended in 100 ul FACS buffer and spun down to wash. To assess the phenotype of CAR T cells, 100 ul of antibody staining cocktail comprised of the following antibodies was added to each well:

CD3 BV785 Biolegend Catalog #317330, Clone OKT3

CD34 PE Invitrogen Catalog # MA1-10205, Clone QBEnd-10

CD4 APC Biolegend Catalog #317416, Clone OKT4

CD8a BV421 Biolegend Catalog #301036, Clone RPA-T8

CD62L BB515 BD Catalog #564742, Clone DREG-56

CD45RO PeCy7 Biolegend Catalog #364230, Clone UCHL1

Cells were re-suspended, covered, and allowed to incubate for 15 minutes at room temperature. The plate was then spun down as above and supernatants were decanted. Cells were washed twice with 100 ul FACS buffer/wash and spun by centrifugation. For flow cytometry, cells were re-suspended in 200 ul FACS buffer and transferred to FACS tubes containing 200 ul pre-aliquoted FACS buffer (total volume 400 ul). Cells were run and data was collected using a BD Fortessa flow cytometer utilizing FACS Diva software.

For analysis, cells were first gated on live populations based on forward scatter and side scatter profiles. CAR T cells were then gated for analysis based on CD3⁻ CD34⁺ expression, which was subsequently applied to a bivariant graph depicting CD8 expression on the y-axis and CD4 expression on the x-axis in order to determine the frequency of CD8 and CD4 T cells with the CAR T cell population.

### Results

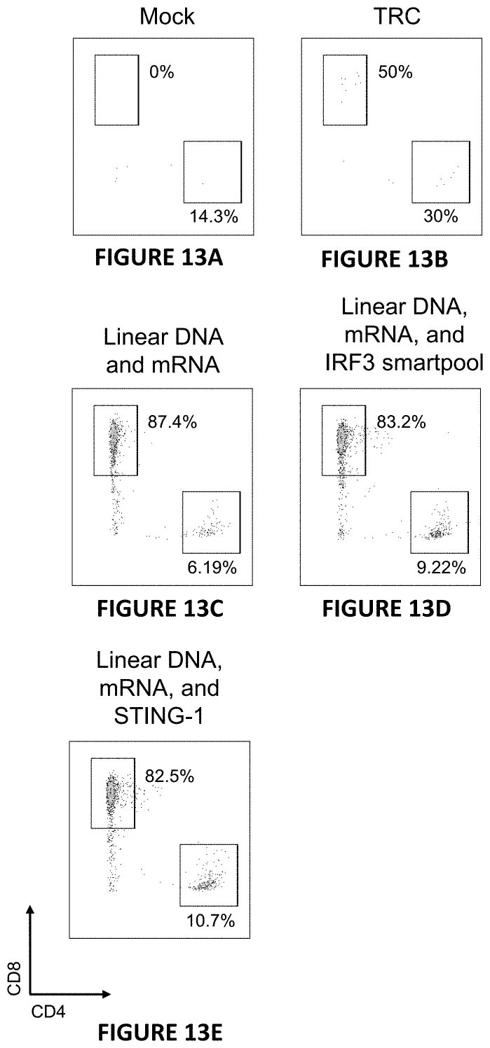

To compare CD4 and CD8 ratios in CAR T cells co-nucleofected with IRF3 or STING smartpool siRNA and linear DNA compared to no siRNA controls, CD3-CD34⁺ T cells were gated on during flow cytometric analysis. CAR T cells were then analyzed on a bivariant graph with showing CD8 and CD4 expression. As in Example 4, few cells from mock nucleofected or T cells nucleofected with mRNA only fell within the pre-defined CAR T cell gate as neither condition was nucleofected with DNA expressing our CAR gene (FIG. 13). Any cells that were collected from these conditions in that gate are representative of non-specific background fluorescence during flow cytometry data collection.

At day 11 post-nucleofection, cells nucleofected with linear DNA and mRNA in the absence of siRNA had a CD8:CD4 ratio of 14.1:1 with 87.4% of CAR T cells expressing CD8 (FIG. 13). By comparison, T cells nucleofected with IRF3 smartpool or STING smartpool siRNA both saw a noteworthy reduction in the CD8:CD4 CAR T cell ratio. Specifically, co-nucleofection with IRF3 smartpool siRNA saw a drop to 9:1 CD8-to-CD4 CAR T cells, while STING smartpool siRNA saw a further reduction to 7.7:1 CD8-to-CD4 CAR T cells (FIG. 13).

### Conclusions

The addition of siRNA specific for either IRF3 or STING during nucleofection of T cells with DNA and mRNA resulted in a lower CD8:CD4 CAR T cell ratio after nucleofection. This alteration in the ratio of CAR T cell subsets would provide a more balanced cell population, thereby allowing for the creation of a longer lasting and more potent product.

### Example 6

### Effect of STING siRNA Smartpools on STING Protein Expression in Primary T Cells

### Purpose

The purpose of this experiment was to determine the effect of the STING siRNA smartpool on STING protein expression in primary human T cells.

### Materials and Methods

5×10⁶ primary human T cells were mock nucleofected or nucleofected with STING smartpool siRNA as described in example 1 and subsequently plated for incubation in 4 ml of T cell media supplemented with IL-2. At 24 post-nucleofection, cells numbers and viabilities were assessed using the Countess II FL (Life Technologies). 1×10⁶ cells from both mock and STING smartpool siRNA conditions were aliquoted into separate 15 ml conical tubes and spun down at 1200 RPM for 5 minutes. Cell supernatants were discarded and cells were immediately snap frozen in a −80° C. freezer. Remaining cells from both conditions were placed back in the incubator.

At 72 hours, cells were counted and 1×10⁶ cells from both mock and STING smartpool siRNA conditions were once again aliquoted into separate 15 ml conical tubes. Cells were spun, supernatants were discarded, and cells were snap frozen in a −80° C. freezer. The remaining T cells from both conditions were spun down in separate 15 ml conical tubes. After decanting the supernatants, cell pellets were re-suspended in 3 ml T cell media supplemented with IL-15 and IL-21 and cells were transferred to a 12-well plate for incubation. Similar to both earlier time points, remaining T cells were counted at 168 hours post-nucleofection (day 7) and 1×10⁶ cells from both mock and STING smartpool siRNA conditions were once again aliquoted into separate 15 ml conical tubes. Cells were spun, supernatants were discarded, and cells were snap frozen in a −80° C. freezer.

Cell pellets from all time points and conditions were thawed, spun down in a microfuge for 5 minutes at 500×g, and washed twice with 1 ml PBS/wash. After the second wash, 100 ul of RIPA buffer supplemented with protease inhibitors were added to each cell pellet and cells were re-suspended. To make cell lysates for western blotting, cells were then freeze thawed 5 times with an average of 10 minutes/cycle. Any remaining debris was briefly spun down using a microfuge and cell lysates were stored at in a −80° C. freezer for future use.

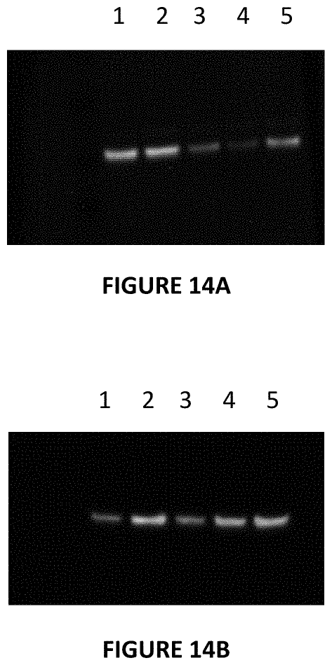

Western blots for STING and β-actin loading control proteins were completed following the manufacturers recommendations. The STING antibody for western blots was purchased from Biolegend (Catalog #675902). Samples in FIG. 14 are as follows: Lane 1, mock-treated, 24 hours; Lane 2, mock-treated, 168 hours; Lane 3, STING siRNA, 24 hours; Lane 4, STING siRNA, 72 hours; Lane 5, STING siRNA, 168 hours.

### Results

In samples acquired at 24 hours post-nucleofection, cell lysates from both mock and STING smartpool siRNA nucleofected cells showed similar expression levels of β-actin by western blot (FIG. 14, lower panel). Interestingly, however, when the same cellular lysates were utilized in western blots probed with an antibody against STING, cell lysates acquired from STING siRNA treated cells 24 hours earlier showed a notable decrease in total STING protein compared to the mock (no siRNA) control cells (FIG. 14, upper panel). Furthermore, cell lysates acquired from STING smartpool siRNA nucleofected cells at 72 hours post-nucleofection expressed a higher level of β-actin, but lower levels of STING protein, compared to both mock and STING treated cells at 24 hours. By comparison, in cell lysates acquired at 168 hours post-nucleofection, β-actin protein levels in mock and STING smartpool siRNA treated samples were similar. Amazingly, total STING protein levels were still reduced in STING smartpool siRNA treated samples compared to mock controls at this time point, but were higher than STING proteins levels detected in samples acquired at 72 hours.

### Conclusions

Nucleofection of primary human T cells with STING smartpool siRNA has a transient and specific effect on STING protein expression, with a peak reduction occurring at 72 hours post-nucleofection and rebounding over time back to baseline after 168 hours post-nucleofection. This reduction in total STING protein levels correlates with decreased cellular cytotoxicity in the presence of cytosolic DNA, resulting in more total, edited, and CAR T cells compared to primary T cells nucleofected in the absence of siRNA.

1. A method for reducing cytotoxicity associated with DNA transfection in primary eukaryotic cells, said method comprising:
(a) reducing interferon regulatory factor 3 (IRF3) signaling in said primary eukaryotic cells; and,
(b) transfecting a DNA template into said primary eukaryotic cells;
wherein said transfected primary eukaryotic cells exhibit improved survival when compared to control cells.

2. A method for increasing the number of gene-edited primary eukaryotic cells following DNA transfection, said method comprising:
(a) reducing interferon regulatory factor 3 (IRF3) signaling in said primary eukaryotic cells;
(b) transfecting a DNA template into said primary eukaryotic cells; and
(c) introducing an endonuclease, or a nucleic acid encoding an endonuclease, into said primary eukaryotic cells;
wherein said endonuclease recognizes and cleaves a recognition sequence present in the genome of said primary eukaryotic cells, and wherein the number of transformed primary eukaryotic cells exhibiting gene editing is increased compared to control cells.

3. A method for increasing gene editing frequency in primary eukaryotic cells following DNA transfection, said method comprising:
(a) reducing interferon regulatory factor 3 (IRF3) signaling in said primary eukaryotic cells;
(b) transfecting a DNA template into said primary eukaryotic cells; and
(c) introducing an endonuclease, or a nucleic acid encoding an endonuclease, into said primary eukaryotic cells;
wherein said endonuclease recognizes and cleaves a recognition sequence present in the genome of said primary eukaryotic cells, and wherein the percentage of transformed primary eukaryotic cells exhibiting gene editing is increased compared to control cells.

4. The method of claim 2 or claim 3, wherein said nucleic acid encoding said endonuclease, or said endonuclease protein, is introduced into said primary eukaryotic cells prior to, simultaneously with, or after transfection with said DNA template.

5. The method of any one of claims 2-4, wherein said nucleic acid encoding said endonuclease is an mRNA or a DNA template.

6. The method of any one of claims 2-5, wherein said endonuclease is an engineered endonuclease.

7. The method of claim 6, wherein said engineered endonuclease is an engineered meganuclease, a TALEN, a zinc finger nuclease (ZFN), a CRISPR/Cas, or a mega-TAL.

8. The method of claim 6 or claim 7, wherein said engineered endonuclease is an engineered meganuclease.

9. The method of any one of claims 2-8, wherein said recognition sequence is located within a gene of interest, and wherein expression of said gene of interest is disrupted and/or activity of a polypeptide encoded by said gene of interest is reduced.

10. A method for increasing the number of primary eukaryotic cells comprising targeted insertion of an exogenous sequence of interest into the genome following DNA transfection, said method comprising:
(a) reducing interferon regulatory factor 3 (IRF3) signaling in said primary eukaryotic cells;
(b) transfecting a DNA template into said primary eukaryotic cells,
wherein said DNA template comprises an exogenous sequence of interest; and
(c) introducing an endonuclease, or a nucleic acid encoding an endonuclease, into said primary eukaryotic cells;
wherein said endonuclease recognizes and cleaves a recognition sequence present in the genome of said primary eukaryotic cells to produce a cleavage site, and wherein said exogenous sequence of interest is flanked by homology arms having homology to regions upstream and downstream of said cleavage site resulting in targeted insertion of said exogenous sequence of interest into said cleavage site by homologous recombination, and wherein the number of transformed primary eukaryotic cells comprising targeted insertion of said exogenous sequence of interest is increased compared to control cells.

11. A method for increasing insertion frequency of an exogenous sequence of interest into the genome in primary eukaryotic cells following DNA transfection, said method comprising:
(a) reducing interferon regulatory factor 3 (IRF3) signaling in said primary eukaryotic cells;
(b) transfecting a DNA template into said primary eukaryotic cells,
wherein said DNA template comprises an exogenous sequence of interest; and
(c) introducing an endonuclease, or a nucleic acid encoding an endonuclease, into said primary eukaryotic cells;
wherein said endonuclease recognizes and cleaves a recognition sequence present in the genome of said primary eukaryotic cells to produce a cleavage site, and wherein said exogenous sequence of interest is flanked by homology arms having homology to regions upstream and downstream of said cleavage site resulting in targeted insertion of said exogenous sequence of interest into said cleavage site by homologous recombination, and wherein the percentage of transformed primary eukaryotic cells comprising targeted insertion of said exogenous sequence of interest is increased compared to control cells.

12. The method of claim 10 or claim 11, wherein said nucleic acid encoding said endonuclease, or said endonuclease protein, is introduced into said primary eukaryotic cells prior to, simultaneously with, or after transfection with said DNA template.

13. The method of any one of claims 10-12, wherein said nucleic acid encoding said endonuclease is an mRNA or a DNA template.

14. The method of any one of claims 10-13, wherein said endonuclease is an engineered endonuclease.

15. The method of claim 14, wherein said engineered endonuclease is an engineered meganuclease, a TALEN, a zinc finger nuclease (ZFN), a CRISPR/Cas, or a mega-TAL.

16. The method of claim 14 or claim 15, wherein said engineered endonuclease is an engineered meganuclease.

17. The method of any one of claims 10-16, wherein said recognition sequence is located within a gene of interest, and wherein expression of said gene of interest is disrupted and/or activity of a polypeptide encoded by said gene of interest is reduced.

18. The method of any one of claims 10-17, wherein said exogenous sequence of interest encodes a chimeric antigen receptor.

19. The method of any one of claims 10-17, wherein said exogenous sequence of interest encodes an exogenous T cell receptor.

20. The method of any one of claims 1-19, wherein said DNA template is a single-stranded DNA template or a double-stranded DNA template.

21. The method of any one of claims 1-20, wherein said DNA template is a plasmid DNA template.

22. The method of any one of claims 1-20, wherein said DNA template is a linearized DNA template.

23. The method of any one of claims 1-22, wherein said DNA template comprises a nucleic acid sequence encoding an exogenous sequence of interest that is expressed in said transfected primary eukaryotic cells.

24. The method of any one of claims 1-23, wherein IRF3 signaling is reduced by downregulating protein expression and/or activity of least one upstream regulator of IRF3.

25. The method of claim 24, wherein IRF3 signaling is reduced by downregulating protein expression and/or activity of STING, TBK1, and/or cGAS.

26. The method of claim 24 or claim 25, wherein IRF3 signaling is reduced by downregulating STING protein expression and/or activity.

27. The method of claim 24 or claim 25, wherein IRF3 signaling is reduced by downregulating cGAS protein expression and/or activity.

28. The method of claim 24 or claim 25, wherein IRF3 signaling is reduced by downregulating TBK1 protein expression and/or activity.

29. The method of any one of claims 1-23, wherein IRF3 signaling is reduced by downregulating IRF3 protein expression and/or activity.

30. The method of any one of claims 24-29, wherein protein expression and/or activity is downregulated by:
(a) RNA interference;
(b) antisense RNA;
(c) gene knockout; or
(d) any combination thereof.

31. The method of any one of claims 24-30, wherein protein expression and/or activity is downregulated by RNA interference.

32. The method of any one of claims 1-23, wherein IRF3 signaling is reduced by small molecule inhibition of at least one upstream regulator of IRF3.

33. The method of claim 32, wherein IRF3 signaling is reduced by small molecule inhibition of stimulator of interferon genes (STING), TANK-binding kinase 1 (TBK1), and/or Cyclic GMP-AMP synthase (cGAS).

34. The method of claim 32 or claim 33, wherein IRF3 signaling is reduced by small molecule inhibition of STING.

35. The method of claim 32 or claim 33, wherein IRF3 signaling is reduced by small molecule inhibition of cGAS.

36. The method of claim 32 or claim 33, wherein IRF3 signaling is reduced by small molecule inhibition of TBK1.

37. The method of any one of claims 1-23, wherein IRF3 signaling is reduced by small molecule inhibition of IRF3.

38. The method of any one of claims 1-37, wherein said primary eukaryotic cells are primary mammalian cells.

39. The method of any one of claims 1-38, wherein said primary eukaryotic cells are primary human, primary non-human primate, primary mouse, primary rat, primary canine, or primary rabbit cells.

40. The method of any one of claims 1-39, wherein said primary eukaryotic cells are primary human cells.

41. The method of claim 40, wherein said primary human cells are primary human T cells.

42. The method of claim 10 or claim 11, wherein:
(a) IRF3 signaling is reduced by downregulating IRF3 protein expression and/or activity using RNA interference;
(b) said nucleic acid encoding an endonuclease is an mRNA encoding an engineered meganuclease;
(c) said exogenous sequence of interest encodes a chimeric antigen receptor and is inserted at said cleavage site by homologous recombination; and
(d) said primary eukaryotic cells are primary human T cells.

43. The method of claim 10 or claim 11, wherein:
(a) IRF3 signaling is reduced by downregulating STING protein expression and/or activity using RNA interference;
(b) said nucleic acid encoding an endonuclease is an mRNA encoding an engineered meganuclease;
(c) said exogenous sequence of interest encodes a chimeric antigen receptor and is inserted at said cleavage site by homologous recombination; and
(d) said primary eukaryotic cells are primary human T cells.

44. A method for high throughput screening of primary human T cells expressing a chimeric antigen receptor (CAR) or exogenous T cell receptor (TCR), said method comprising:
(a) reducing interferon regulatory factor 3 (IRF3) signaling in said primary human T cells;
(b) transfecting a DNA template into said primary human T cells, wherein said DNA template comprises an exogenous nucleic acid sequence encoding a CAR or an exogenous TCR;
(c) introducing an endonuclease, or a nucleic acid encoding an endonuclease, into said primary human T cells, wherein said endonuclease recognizes and cleaves a recognition sequence present in the genome of said primary human T cells to produce a cleavage site, and wherein said exogenous nucleic acid sequence is inserted at said cleavage site, and wherein said CAR or said exogenous TCR is expressed on the cell surface; and
(d) characterizing a phenotype of said primary human T cells expressing said CAR or said exogenous TCR.

45. The method of claim 44, wherein said DNA template is a single-stranded DNA template or a double-stranded DNA template.

46. The method of claim 44 or claim 45, wherein said DNA template is a plasmid DNA template.

47. The method of claim 44, wherein said DNA template is a linearized DNA template.

48. The method of any one of claims 44-47, wherein IRF3 signaling is reduced by downregulating protein expression and/or activity of least one upstream regulator of IRF3.

49. The method of claim 48, wherein IRF3 signaling is reduced by downregulating protein expression and/or activity of STING, TBK1, and/or cGAS.

50. The method of claim 48 or claim 49, wherein IRF3 signaling is reduced by downregulating STING protein expression and/or activity.

51. The method of claim 48 or claim 49, wherein IRF3 signaling is reduced by downregulating cGAS protein expression and/or activity.

52. The method of claim 48 or claim 49, wherein IRF3 signaling is reduced by downregulating TBK1 protein expression and/or activity.

53. The method of any one of claims 44-47, wherein IRF3 signaling is reduced by downregulating IRF3 protein expression and/or activity.

54. The method of any one of claims 48-53, wherein protein expression and/or activity is downregulated by:
(a) RNA interference (siRNA or shRNA);
(b) antisense RNA;
(c) gene knockout; or
(d) any combination thereof.

55. The method of any one of claims 48-54, wherein protein expression and/or activity is downregulated by RNA interference.

56. The method of any one of claims 44-47, wherein IRF3 signaling is reduced by small molecule inhibition of at least one upstream regulator of IRF3.

57. The method of claim 56, wherein IRF3 signaling is reduced by small molecule inhibition of stimulator of interferon genes (STING), TANK-binding kinase 1 (TBK1), and/or Cyclic GMP-AMP synthase (cGAS).

58. The method of claim 56 or claim 57, wherein IRF3 signaling is reduced by small molecule inhibition of STING.

59. The method of claim 56 or claim 57, wherein IRF3 signaling is reduced by small molecule inhibition of cGAS.

60. The method of claim 56 or claim 57, wherein IRF3 signaling is reduced by small molecule inhibition of TBK1.

61. The method of any one of claims 44-47, wherein IRF3 signaling is reduced by small molecule inhibition of IRF3.

62. The method of any one of claims 44-61, wherein said nucleic acid encoding said endonuclease, or said endonuclease protein, is introduced into said primary eukaryotic cells prior to, simultaneously with, or after transfection with said DNA template.

63. The method of any one of claims 44-62, wherein said nucleic acid encoding said endonuclease is an mRNA or a DNA template.

64. The method of any one of claims 44-63, wherein said endonuclease is an engineered endonuclease.

65. The method of claim 64, wherein said engineered endonuclease is an engineered meganuclease, a TALEN, a zinc finger nuclease (ZFN), a CRISPR/Cas, or a mega-TAL.

66. The method of claim 64 or claim 65, wherein said engineered endonuclease is an engineered meganuclease.

67. The method of any one of claims 44-66, wherein said recognition sequence is located within a gene of interest, and wherein expression of said gene of interest is disrupted and/or activity of a polypeptide encoded by said gene of interest is reduced.

68. A method for high throughput screening of primary human T cells expressing a chimeric antigen receptor (CAR) or exogenous T cell receptor (TCR), said method comprising:
(a) reducing STING signaling in said primary human T cells;
(b) transfecting a DNA template into said primary human T cells, wherein said DNA template comprises an exogenous nucleic acid sequence encoding a CAR or an exogenous TCR;
(c) introducing an endonuclease, or a nucleic acid encoding an endonuclease, into said primary human T cells, wherein said endonuclease recognizes and cleaves a recognition sequence present in the genome of said primary human T cells to produce a cleavage site, and wherein said exogenous nucleic acid sequence is inserted at said cleavage site, and wherein said CAR or said exogenous TCR is expressed on the cell surface; and
(d) characterizing a phenotype of said primary human T cells expressing said CAR or said exogenous TCR.

69. The method of claim 68, wherein said DNA template is a single-stranded DNA template or a double-stranded DNA template.

70. The method of claim 68 or claim 69, wherein said DNA template is a plasmid DNA template.

71. The method of claim 68, wherein said DNA template is a linearized DNA template.

72. The method of any one of claims 68-71, wherein STING signaling is reduced by downregulating STING protein expression and/or activity.

73. The method of claim 72, wherein STING protein expression and/or activity is downregulated by:
(a) RNA interference (siRNA or shRNA);
(b) antisense RNA;
(c) gene knockout; or
(d) any combination thereof.

74. The method of claim 72 or claim 73, wherein STING protein expression and/or activity is downregulated by RNA interference.

75. The method of any one of claims 68-71, wherein STING signaling is reduced by small molecule inhibition.

76. The method of any one of claims 68-75, wherein said nucleic acid encoding said endonuclease, or said endonuclease protein, is introduced into said primary eukaryotic cells prior to, simultaneously with, or after transfection with said DNA template.

77. The method of any one of claims 68-76, wherein said nucleic acid encoding said endonuclease is an mRNA or a DNA template.

78. The method of any one of claims 68-77, wherein said endonuclease is an engineered endonuclease.

79. The method of claim 78, wherein said engineered endonuclease is an engineered meganuclease, a TALEN, a zinc finger nuclease (ZFN), a CRISPR/Cas, or a mega-TAL.

80. The method of claim 78 or claim 79, wherein said engineered endonuclease is an engineered meganuclease.

81. The method of any one of claims 68-80, wherein said recognition sequence is located within a gene of interest, and wherein expression of said gene of interest is disrupted and/or activity of a polypeptide encoded by said gene of interest is reduced.

82. The method of any one of claims 44-81, wherein said step of characterizing a phenotype comprises determining the frequency of cell surface expression of said CAR or said exogenous TCR.

83. The method of any one of claims 44-82, wherein said step of characterizing a phenotype comprises determining the memory phenotype of said primary human T cells expressing a CAR or an exogenous TCR.

84. The method of any one of claims 44-83, wherein said step of characterizing a phenotype comprises determining the CD4⁺ to CD8⁺ ratio of said primary human T cells expressing a CAR or an exogenous TCR.

85. The method of any one of claims 44-84, wherein said step of characterizing a phenotype comprises quantifying exhaustion markers expressed by said primary human T cells expressing a CAR or an exogenous TCR.

86. The method of claim 85, wherein said exhaustion markers include TIM-3, PD-1, and/or LAG-3.

87. The method of any one of claims 44-86, wherein said step of characterizing a phenotype comprises quantifying antigen-independent and/or antigen-induced secretion of cytokines by said primary human T cells expressing a CAR or an exogenous TCR.

88. The method of claim 87, wherein said cytokines include interferon-gamma, interleukin-2, tumor necrosis factor alpha, granzyme B, and perforin.

89. The method of any one of claims 44-88, wherein said step of characterizing a phenotype comprises determining antigen-independent phosphorylated protein expression by said primary human T cells expressing a CAR or an exogenous TCR.

90. The method of claim 89, wherein said phosphorylated protein can include CD3ζ, extracellular signal-regulated kinase (ERK), RAC-alpha serine/threonine-protein kinase (AKT1), and p38.

91. The method of any one of claims 44-90, wherein said step of characterizing a phenotype comprises evaluating cytotoxicity of said primary human T cells expressing a CAR or an exogenous TCR against antigen-bearing target cells.

92. The method of any one of claims 44-91, wherein said step of characterizing a phenotype comprises determining antigen-independent and/or antigen-induced proliferative capacity of said primary human T cells expressing a CAR or an exogenous TCR.

93. The method of any one of claims 44-92, wherein said step of characterizing a phenotype comprises evaluating exhaustion of said primary human T cells expressing a CAR or an exogenous TCR after repeated antigen encounter.

94. A genetically-modified primary eukaryotic cell prepared by the method of any one of claims 1-93.

95. The genetically-modified primary eukaryotic cell of claim 94, wherein said cell is a genetically-modified primary human T cell.

96. The genetically-modified primary eukaryotic cell of claim 94 or claim 95, wherein said cell expresses a chimeric antigen receptor or an exogenous T cell receptor.

97. A genetically-modified primary eukaryotic cell comprising a nucleic acid sequence encoding a chimeric antigen receptor or an exogenous T cell receptor, wherein IRF3 signaling is reduced in said cell when compared to a control cell.

98. The genetically-modified primary eukaryotic cell of claim 97, wherein IRF3 protein expression and/or activity is reduced when compared to a control cell.

99. The genetically-modified primary eukaryotic cell of claim 97, wherein STING protein expression and/or activity is reduced when compared to a control cell.

100. The genetically-modified primary eukaryotic cell of claim 97, wherein TBK1 protein expression and/or activity is reduced when compared to a control cell.

101. The genetically-modified primary eukaryotic cell of claim 97, wherein cGAS protein expression and/or activity is reduced when compared to a control cell.

102. A genetically-modified primary eukaryotic cell comprising a nucleic acid sequence encoding a chimeric antigen receptor or an exogenous T cell receptor, wherein STING signaling is reduced in said cell when compared to a control cell.

103. The genetically-modified primary eukaryotic cell of claim 102, wherein STING protein expression and/or activity is reduced when compared to a control cell.

104. The genetically-modified primary eukaryotic cell of any one of claims 97-103, wherein said cell is a genetically-modified primary human T cell.


SEQUENCE LISTING (9 sequences)

SEQ ID NO: 1 - protein, 9 aa, Chlamydomonas reinhardtii
LAGLIDADG

SEQ ID NO: 2 - RNA, 19 nt, Artificial Sequence
  Synthetic polynucleotide
cgaggccacuggugcauau

SEQ ID NO: 3 - RNA, 19 nt, Artificial Sequence
  Synthetic polynucleotide
ccagacaccucuccggaca

SEQ ID NO: 4 - RNA, 19 nt, Artificial Sequence
  Synthetic polynucleotide
ggagugaugagcuacguga

SEQ ID NO: 5 - RNA, 19 nt, Artificial Sequence
  Synthetic polynucleotide
agacauucuggaugaguua

SEQ ID NO: 6 - RNA, 19 nt, Artificial Sequence
  Synthetic polynucleotide
ucauaaacuuuggaugcua

SEQ ID NO: 7 - RNA, 19 nt, Artificial Sequence
  Synthetic polynucleotide
cgaacucucucaaugguau

SEQ ID NO: 8 - RNA, 19 nt, Artificial Sequence
  Synthetic polynucleotide
agcugggacugcuguuaaa

SEQ ID NO: 9 - RNA, 19 nt, Artificial Sequence
  Synthetic polynucleotide
gcagaugacagcagcuucu